(19) United States
(12) Patent Application Publication
(10) Pub. No.: US 2025/0360142 A1
(43) Pub. Date: Nov. 27, 2025

(54) NOVEL ATP-SENSITIVE POTASSIUM CHANNEL POTENTIATORS, THEIR PREPARATION AND USE

(71) Applicant: RHYTHM PHARMACEUTICALS, INC., Boston, MA (US)

(72) Inventors: Piet Wigerinck, Aartselaar (BE); Claudine Van Der Sande, Amsterdam (NL); Bertrand Vivet, ACHENHEIM (FR); Laurence Anne Mevellec, LOUVIERS (FR)

(21) Appl. No.: 19/122,722

(22) PCT Filed: Oct. 18, 2023
(86) PCT No.: PCT/US2023/035454

§ 371 Date: Apr. 18, 2025

(30) Foreign Application Priority Data
Oct. 18, 2022 (NL) N2033343

(51) Int. Cl. A61K 31/549; C07D 285/24

(52) U.S. Cl. CPC A61K 31/549; C07D 285/24

(57) ABSTRACT

The present disclosure relates to compounds of general formula (I) or a pharmaceutically acceptable salt, solvate and/or hydrate thereof, methods for their preparation, the use of said compounds for use as a medicament, and their use in the treatment and/or prevention of a disease or disorder, such as a metabolic disorder (e.g., hyperinsulinism, e.g., congenital hyperinsulinism). The present disclosure also relates to a pharmaceutical composition comprising a compound of general formula (I) and a pharmaceutically acceptable carrier and its use as a medicament, in particular its use in the treatment of a disease or disorder, such as a metabolic disorder (e.g., hyperinsulinism, e.g., congenital hyperinsulinism).

### CLAIM OF PRIORITY

This application claims priority to Dutch Patent Application No. N2033343, filed Oct. 18, 2022. The entire contents of the foregoing application is incorporated herein by reference in its entirety.

### TECHNICAL FIELD

The invention relates to compounds of general formula (I) or a pharmaceutically acceptable salt, solvate and/or hydrate thereof, methods for their preparation, the use of said compounds for use as a medicament, and their use in the treatment and/or prevention of a disease or disorder. In an embodiment, the disease or disorder is hyperinsulinism, in particular congenital hyperinsulinism. The invention also relates to a pharmaceutical composition comprising a compound of general formulas (I) and a pharmaceutically acceptable carrier and its use as a medicament, in particular its use in the treatment of a disease or disorder, such as hyperinsulinism or congenital hyperinsulinism.

### BACKGROUND OF THE INVENTION

K_{ATP} channels are composed of four pore-forming inward rectifier potassium channel subunits which are Kir6.1 or Kir6.2 and four regulatory sulfonylurea receptor subunits SUR1 or SUR2. The assembly of these subunits in different combinations result in tissue specific K_{ATP} channel isoforms. For instance, the pancreatic p-cell type K_{ATP} channels have the combination of SUR1 with the Kir6.2 subunits (SUR1/Kir6.2). SUR2A/Kir6.2 and SUR2B/Kir6.2 or Kir6.1 combinations are present in cardiac and smooth muscles, respectively. Each SUR subunit comprises 17 transmembrane domain clustered into three regions and two intracellular nucleotide binding (NBD). The Kir6.x comprises two membrane spinning domains which provides a K⁻ selectivity P-loop.

In some embodiments, K_{ATP} channels (SUR1/Kir6.2) are closed in certain patients due their dysfunctionality. Moreover, β-cells are electrically active which leads to an inappropriate insulin release independent from the plasma glucose level. The treatment strategy is therefore opening K_{ATP} channels to turn β-cells into inactive state again by using K_{ATP} channel openers (KCOs). KCOs provide β-cell rest by hyperpolarizing the cell membrane and inhibiting calcium entry into the cell.

KCOs have previously been used to inhibit insulin secretion and were therefore used in the treatment of type 1 and type 2 diabetes or reduction of blood pressure. They are also used in the treatment of CHI patients. However, known KCOs were not tissue specific due to ubiquitous distribution of the K_{ATP} channels which results in undesired side effects. Therefore, the use of KCOs has previously been limited due to their moderate potency and limited selectivity.

Currently available KCOs comprise first-generation benzopyrans, benzothiadiazines, cyanoguanidines, pyridyl nitrates and thioformamides, and second-generation cyclobutenediones, dihydropyridines and tertiarycarbinols.

Diazoxide, a benzothiadizine, is a K_{ATP} channel-opener, a first-line drug treatment for patients with CHI. Diazoxide was first approved for medical use in 1973 and it is used for the treatment of acute hypertension as a vasodilator and also used in the treatment of low blood sugar. However, diazoxide is not tissue specific and is effective mainly on the SUR1, moreover its activity shifts to SUR2-containing channels in the presence of intracellular MgADP. The side effects of diazoxide are numerous like Na+ and fluid retention, hypertrichosis and loss of appetite including the life-threatening such as cardiac failure, pulmonary hypertension, hyperuricemia, bone marrow, suppression, and anemia. The vasodilating effects of diazoxide are mediated by its binding and activation of SUR2-containing K_{ATP} channels resulting in membrane hyperpolarization and reduced Ca²⁻ influx in arterial smooth muscle cells. It has recently been shown that, in addition to the beta-cell K_{ATP} channel, diazoxide can activate K_{ATP} channels in peripheral tissues which contain the SUR2 subunit. This may account for many of the off-target side effects of diazoxide, including salt and water retention, hypertrichosis, bitter taste, and, rarely, pulmonary hypertension.

Additionally, without being bound by theory, diazoxide may only be effective when K_{ATP} channels are functional but most of the mutations in ABCC8 and KCNJ11 genes. This means that the most severe cases of CHI in patients may not be responsive to diazoxide. People suffer from milder versions of the condition are somewhat responsive to diazoxide, but do suffer from the side effects mentioned above. Therefore, treatment with diazoxide may still result in life threatening hypoglycaemia in one third of the responsive patients.

Alternative treatments to diazoxide and to other KCOs include the administration of other medications like glucagon, somatostatin analogues, nifedipine, GLP1-receptor antagonists and sirolimus which mainly acts by lowering Ca²⁺ influx into β-cells. However, these drugs also display many side effects such as gastrointestinal symptoms, formation of gall stones, suppression of pituitary hormones, necrotizing enterocolitis, hypotension, immune suppression, thrombocytosis, impaired immune response.

Other treatment methods of CHI involve surgical treatment, which is carried out when drug treatment is not sufficient. Partial or near-total pancreatectomy can be considered depending on whether the CHI is focal or diffuse and drug-unresponsive which clearly requires a surgical intervention.

Further treatment methods involve modifying the insulin levels which are already present in the plasma. Said methods target the insulin downstream pathways and involve molecules acting as insulin antagonists or insulin receptor antagonists which can cause further undesired effects.

These molecules are usually administered via injection and it is not a preferred method since children suffering from any CHI can be overweighed and difficult to puncture.

Hence, a need remains for the provision of potent and well-tolerated compounds which can selectively bind to SUR1/Kir6.2 and inhibit glucose stimulated insulin secretion.

### SUMMARY OF THE INVENTION

A first aspect of the present disclosure relates to a compound having a structure of

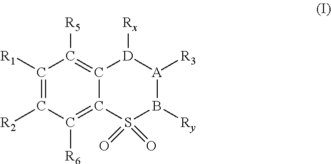

or a pharmaceutically acceptable salt, tautomer, stereoisomer, hydrate, or isotope thereof, wherein A is C(R′); each of B and D are independently N; wherein A and B or A and D are connected via a double or single bond; each of R₁, R₂, R₅, and R₆ are independently hydrogen, C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano; wherein A and B form a double bond and Rₓ is hydrogen or wherein A and D form a double bond and R_{y} is hydrogen; R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), wherein alkyl, alkenyl, alkynyl, heteroalkyl, haloalkyl, cycloalkyl, and heterocyclyl are each optionally substituted with one or more R₄; R′ is absent, hydrogen, or C1-C6 alkyl; each R₄ is independently C1-C6 alkyl, C1-C6 heteroalkyl, C1-C6 haloalkyl, cycloalkyl, heterocyclyl, fluorine, chlorine, bromine, or iodine; R^{A} is hydrogen, C1-C6 alkyl, C2-C6 alkenyl, C1-C6 alkynyl, C1-C6 heteroalkyl, C1-C6 haloalkyl, cycloalkyl, or heterocyclyl, wherein alkyl, alkenyl, alkynyl, heteroalkyl, haloalkyl, cycloalkyl, and heterocyclyl are each optionally substituted with one or more R₇; each of R^{B} and R^{C} is independently hydrogen, C1-C6 alkyl, C2-C6 alkenyl, C1-C6 alkynyl, C1-C6 heteroalkyl, C1-C6 haloalkyl, cycloalkyl, heterocyclyl, wherein alkyl, alkenyl, alkynyl, heteroalkyl, haloalkyl, cycloalkyl, and heterocyclyl are each optionally substituted with one or more R₇; or wherein R^{B} and R^{C} are taken together to form heterocyclyl optionally substituted with one or more R⁷; each R⁷ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano.

In an embodiment, B is N. In an embodiment, D is N. In an embodiment, A is C. In an embodiment, R₅ is hydrogen. In an embodiment, R₆ is hydrogen. In an embodiment, each of R₁ and R₂ is independently hydrogen, fluorine, chlorine, bromine, iodine, nitro, or cyano. In an embodiment, R₁ is hydrogen, fluorine, chlorine, bromine, iodine, nitro, or cyano. In an embodiment, R₁ is hydrogen, fluorine, chlorine, bromine, iodine, cyano. In an embodiment, R₁ is hydrogen, fluorine or chlorine. In an embodiment, R₁ is hydrogen. In an embodiment, R₁ is fluorine. In an embodiment, R₁ is chlorine. In an embodiment, R₁ is bromine. In an embodiment, R₁ is iodine. In an embodiment, R₁ is cyano. In an embodiment, R₂ is hydrogen, fluorine, chlorine, bromine, iodine, nitro, or cyano. In an embodiment, R₂ is hydrogen, fluorine, chlorine, bromine, iodine, cyano. In an embodiment, R₂ is hydrogen, fluorine or chlorine. In an embodiment, R₂ is hydrogen. In an embodiment, R₂ is fluorine. In an embodiment, R₂ is chlorine. In an embodiment, R₂ is bromine. In an embodiment, R₁ is iodine. In an embodiment, R₂ is cyano.

In an embodiment, R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), each of which is optionally substituted with optionally substituted with one or more R₇. In an embodiment, R₃ is C1-C6 heteroalkyl optionally substituted with optionally substituted with one or more R₇. In an embodiment, R₃ is C1-C6 haloalkyl, optionally substituted with optionally substituted with one or more R₇. In an embodiment, R₃ is cycloalkyl, optionally substituted with optionally substituted with one or more R₇. In an embodiment, R₃ heterocyclyl, optionally substituted with optionally substituted with one or more R₇.

In an embodiment, R₃ is N(R^{B})(R^{C}). In an embodiment, R^{B} and R^{C} are taken together to form heterocyclyl optionally substituted with one or more R⁷ (e.g., a 4-membered heterocyclyl, 5-membered heterocyclyl, 6-membered heterocyclyl, or a bridged heterocyclyl). In an embodiment, R^{B} is hydrogen. In an embodiment, R^{C} is C1-C6 alkyl, C2-C6 alkenyl, C1-C6 alkynyl, C1-C6 heteroalkyl, C1-C6 haloalkyl, cycloalkyl, heterocyclyl, each optionally substituted with one or more R₇. In an embodiment, R^{C} is C1-C6 alkyl optionally substituted with one or more R₇. In an embodiment, R^{C} is C1-C6 heteroalkyl optionally substituted with one or more R₇. In an embodiment, R^{C} is C1-C6 haloalkyl optionally substituted with one or more R₇. In an embodiment, R^{C} is cycloalkyl optionally substituted with one or more R₇ (e.g., monocyclic cycloalkyl, bicyclic cycloalkyl). In an embodiment, R^{C} is cycloalkyl substituted with one or more R₇, wherein R₇ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano. In an embodiment, R^{C} is heterocyclyl optionally substituted with one or more R₇.

In an embodiment, the compound of Formula (I) is not 3-(tert-butylamino)-7-chloro-4H-benzo[e][1,2,4]thiadiazine 1,1-dioxide or a pharmaceutically acceptable salt thereof.

In an embodiment, the compound of Formula (I) is a compound of Formula (I-a):

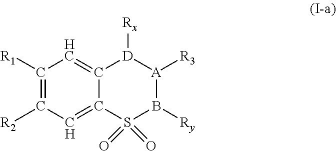

or a pharmaceutically acceptable salt, tautomer, stereoisomer, hydrate, or isotope thereof, wherein A is C; each of B and D are independently N; wherein A and B or A and D are connected via a double or single bond; R₁ and R₂ are hydrogen, fluorine, chlorine, bromine and iodine; and wherein A and B form a double bond and Rₓ is H or wherein A and D form a double bond and R_{y} is H; R₃ is independently selected from the group consisting of:

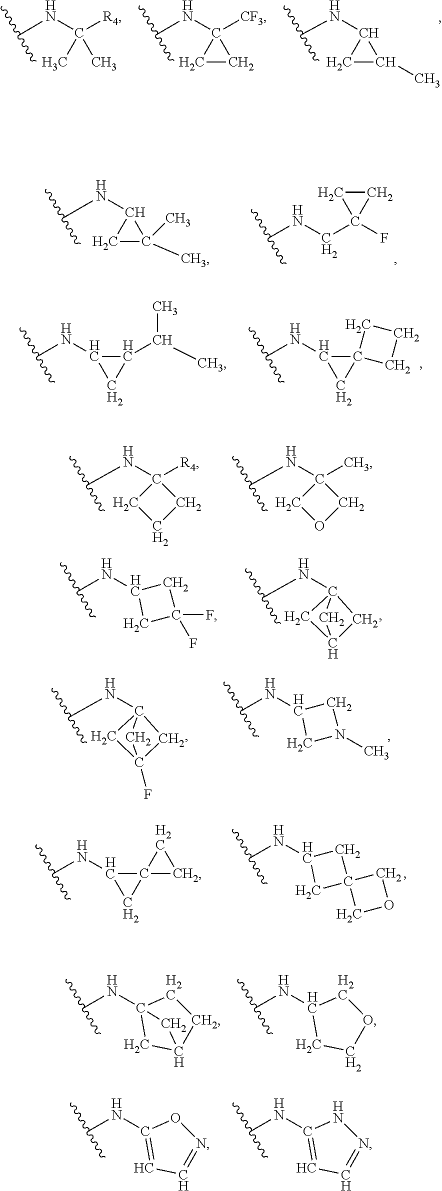

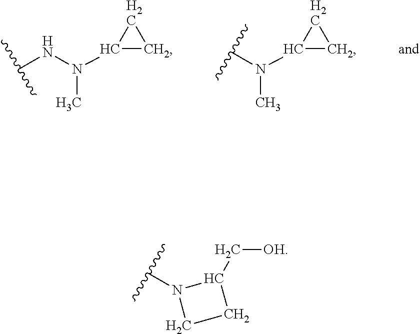

R₄ is selected from the group consisting of C1-C3 alkyl optionally substituted with one to two substituents selected from the group consisting of fluoro and hydroxy, ethynyl, C1-C2 alkoxy, (methoxy)-C1-C2 alkyl, cyano, fluoro, (methylsulfonyl)-C1-C2 alkyl, (dimethylamino)-C1-C2 alkyl and n-methyl carbamoyl, under the proviso that said compound is not 3-(tert-butylamino)-7-chloro-4H-benzo[e][1,2,4]thiadiazine 1,1-dioxide.

The inventors have surprisingly found that the compounds described herein exhibit remarkable potency for binding selectively to SUR1/Kir6.2 and inhibiting glucose stimulated insulin secretion. Without being bound by theory, as the binding of these compounds to the target is specific, reduced off target effects are observed. Moreover, the compounds according to the present invention exhibit excellent bioavailability, stability and safety and can be administered orally.

The present invention includes all tautomeric forms, metabolites or prodrugs of the compounds of the present invention. The present invention further includes unsolvated forms, solvated forms including hydrated forms of the compounds of the present invention.

A second aspect of the present invention relates to a process for preparing compounds according to formula (I).

A third aspect of the present invention relates to a pharmaceutical composition comprising a therapeutically active amount of a compound according to formula (I) and a pharmaceutically acceptable carrier.

A last aspect of the present invention relates to the use of these compounds are pharmaceutical compositions thereof as a medicament, in particular for use in the treatment of a disease or a disorder, e.g., a metabolic disorder (e.g., methyperinsulinism (HI) or congenital hyperinsulinism (CHI)), a cancer, or a neurological disorder, or a cardiovascular disorder.

### BRIEF DESCRIPTION OF THE DRAWINGS

FIG. 1 illustrates the inhibition of insulin by diazoxide in the GSIS assay.

FIG. 2 illustrates the inhibition of insulin by Compound 42 in the GSIS assay.

FIG. 3 illustrates the inhibition of insulin by Compound 54 in the GSIS assay.

### DETAILED DESCRIPTION OF THE INVENTION

Described herein are compounds, compositions, and related uses and preparations thereof for the treatment of a disease or disorder, such as CHI. In a first aspect, the present disclosure features a compound of Formula (I):

A first aspect of the present disclosure relates to a compound having a structure of Formula (I):

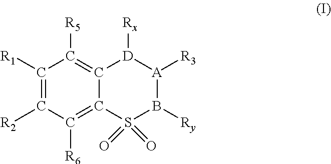

or a pharmaceutically acceptable salt, tautomer, stereoisomer, hydrate, or isotope thereof, wherein A is C(R′); each of B and D are independently N; wherein A and B or A and D are connected via a double or single bond; each of R₁, R₂, R₅, and R₆ are independently hydrogen, C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano; wherein A and B form a double bond and Rₓ is hydrogen or wherein A and D form a double bond and R_{y} is hydrogen; R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), wherein alkyl, alkenyl, alkynyl, heteroalkyl, haloalkyl, cycloalkyl, and heterocyclyl are each optionally substituted with one or more R₄; R′ is absent, hydrogen, or C1-C6 alkyl; each R₄ is independently C1-C6 alkyl, C1-C6 heteroalkyl, C1-C6 haloalkyl, cycloalkyl, heterocyclyl, fluorine, chlorine, bromine, or iodine; R^{A} is hydrogen, C1-C6 alkyl, C2-C6 alkenyl, C1-C6 alkynyl, C1-C6 heteroalkyl, C1-C6 haloalkyl, cycloalkyl, or heterocyclyl, wherein alkyl, alkenyl, alkynyl, heteroalkyl, haloalkyl, cycloalkyl, and heterocyclyl are each optionally substituted with one or more R₇; each of R^{B} and R^{C} is independently hydrogen, C1-C6 alkyl, C2-C6 alkenyl, C1-C6 alkynyl, C1-C6 heteroalkyl, C1-C6 haloalkyl, cycloalkyl, heterocyclyl, wherein alkyl, alkenyl, alkynyl, heteroalkyl, haloalkyl, cycloalkyl, and heterocyclyl are each optionally substituted with one or more R₇; or wherein R^{B} and R^{C} are taken together to form heterocyclyl optionally substituted with one or more R⁷; each R⁷ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano.

In an embodiment, B is N. In an embodiment, D is N. In an embodiment, A is C. In an embodiment, R₅ is hydrogen. In an embodiment, R₆ is hydrogen. In an embodiment, each of R₁ and R₂ is independently hydrogen, fluorine, chlorine, bromine, iodine, nitro, or cyano. In an embodiment, R₁ is hydrogen, fluorine, chlorine, bromine, iodine, nitro, or cyano. In an embodiment, R₁ is hydrogen, fluorine, chlorine, bromine, iodine, cyano. In an embodiment, R₁ is hydrogen, fluorine or chlorine. In an embodiment, R₁ is hydrogen. In an embodiment, R₁ is fluorine. In an embodiment, R₁ is chlorine. In an embodiment, R₁ is bromine. In an embodiment, R₁ is iodine. In an embodiment, R₁ is cyano. In an embodiment, R₂ is hydrogen, fluorine, chlorine, bromine, iodine, nitro, or cyano. In an embodiment, R₂ is hydrogen, fluorine, chlorine, bromine, iodine, cyano. In an embodiment, R₂ is hydrogen, fluorine or chlorine. In an embodiment, R₂ is hydrogen. In an embodiment, R₂ is fluorine. In an embodiment, R₂ is chlorine. In an embodiment, R₂ is bromine. In an embodiment, R₁ is iodine. In an embodiment, R₂ is cyano.

In an embodiment, R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), each of which is optionally substituted with optionally substituted with one or more R₇. In an embodiment, R₃ is C1-C6 heteroalkyl optionally substituted with optionally substituted with one or more R₇. In an embodiment, R₃ is C1-C6 haloalkyl, optionally substituted with optionally substituted with one or more R₇. In an embodiment, R₃ is cycloalkyl, optionally substituted with optionally substituted with one or more R₇. In an embodiment, R₃ heterocyclyl, optionally substituted with optionally substituted with one or more R₇.

In an embodiment, R₃ is N(R^{B})(R^{C}). In an embodiment, R^{B} and R^{C} are taken together to form heterocyclyl optionally substituted with one or more R⁷ (e.g., a 4-membered heterocyclyl, 5-membered heterocyclyl, 6-membered heterocyclyl, or a bridged heterocyclyl). In an embodiment, R^{B} is hydrogen. In an embodiment, R^{C} is C1-C6 alkyl, C2-C6 alkenyl, C1-C6 alkynyl, C1-C6 heteroalkyl, C1-C6 haloalkyl, cycloalkyl, heterocyclyl, each optionally substituted with one or more R₇. In an embodiment, R^{C} is C1-C6 alkyl optionally substituted with one or more R₇. In an embodiment, R^{C} is C1-C6 heteroalkyl optionally substituted with one or more R₇. In an embodiment, R^{C} is C1-C6 haloalkyl optionally substituted with one or more R₇. In an embodiment, R^{C} is cycloalkyl optionally substituted with one or more R₇ (e.g., monocyclic cycloalkyl, bicyclic cycloalkyl). In an embodiment, R^{C} is cycloalkyl substituted with one or more R₇, wherein R₇ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano. In an embodiment, R^{C} is heterocyclyl optionally substituted with one or more R₇.

In an embodiment, the compound of Formula (I) is not 3-(tert-butylamino)-7-chloro-4H-benzo[e][1,2,4]thiadiazine 1,1-dioxide or a pharmaceutically acceptable salt thereof.

In an embodiment, the compound of Formula (I) is a compound of Formula (I-a):

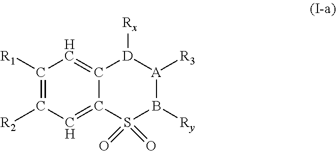

or a pharmaceutically acceptable salt, tautomer, stereoisomer, hydrate, or isotope thereof, wherein A is C; each of B and D are independently N; wherein A and B or A and D are connected via a double or single bond; R₁ and R₂ are hydrogen, fluorine, chlorine, bromine and iodine; and wherein A and B form a double bond and Rₓ is H or wherein A and D form a double bond and R_{y} is H; R₃ is independently selected from the group consisting of:

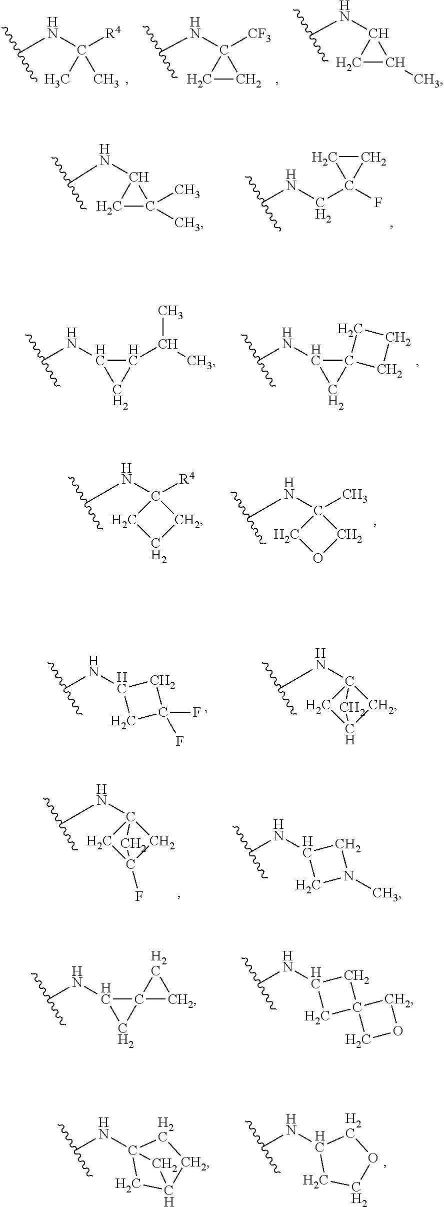

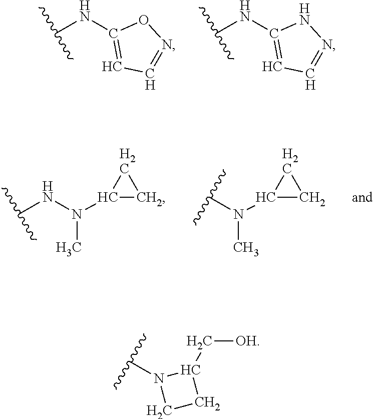

R₄ is selected from the group consisting of C1-C3 alkyl optionally substituted with one to two substituents selected from the group consisting of fluoro and hydroxy, ethynyl, C1-C2 alkoxy, (methoxy)-C1-C2 alkyl, cyano, fluoro, (methylsulfonyl)-C1-C2 alkyl, (dimethylamino)-C1-C2 alkyl and n-methyl carbamoyl, under the proviso that said compound is not 3-(tert-butylamino)-7-chloro-4H-benzo[e][1,2,4]thiadiazine 1,1-dioxide. In relation to these compounds the present inventors have surprisingly found that these compounds exhibit remarkable potency for binding selectively to SUR1/Kir6.2 and inhibiting glucose stimulated insulin secretion. In view of the fact that the binding of these compounds to the target is so specific, reduced off target effects are observed. Moreover, the compounds according to the present invention exhibit excellent bioavailability, stability and safety and can be administered orally.

In another embodiment, the compound of Formula (I) is a compound of Formula (I-b):

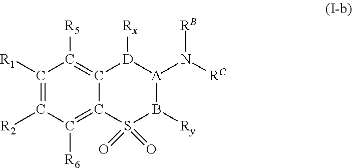

or a pharmaceutically acceptable salt, tautomer, stereoisomer, hydrate, or isotope thereof, wherein A, B, D, R₁, R₂, R₅, R₆, Rₓ R_{y}, R^{A}, R^{B}, R^{C}, and R⁷ are defined as provided for Formula (I).

In another embodiment, the compound of Formula (I) is a compound of Formula (I-c):

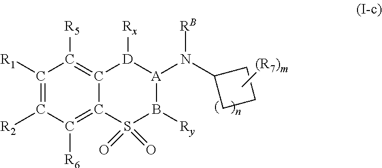

or a pharmaceutically acceptable salt, tautomer, stereoisomer, hydrate, or isotope thereof, wherein A, B, D, R₁, R₂, R₅, R₆, Rₓ R_{y}, R^{A}, R^{B}, R^{C}, and R⁷ are defined as provided for Formula (I), m is an integer between 0 and 12; and n is an integer between 0 and 4.

In another embodiment, the compound of Formula (I) is a compound of Formula (I-d):

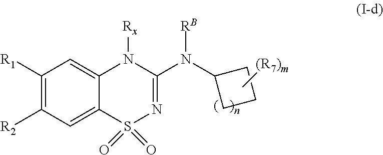

or a pharmaceutically acceptable salt, tautomer, stereoisomer, hydrate, or isotope thereof, wherein R₁, R₂, R₅, Rₓ, and R⁷ are defined as provided for Formula (I), m is an integer between 0 and 12; and n is an integer between 0 and 4.

In another embodiment, the compound of Formula (I) is a compound of Formula (I-e):

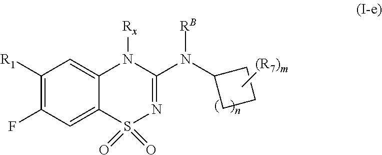

or a pharmaceutically acceptable salt, tautomer, stereoisomer, hydrate, or isotope thereof, wherein R₁, R₅, Rₓ, and R⁷ are defined as provided for Formula (I), m is an integer between 0 and 12; and n is an integer between 0 and 4.

In another embodiment, the compound of Formula (I) is a compound of Formula (I-f):

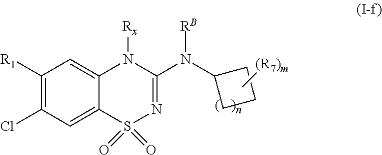

or a pharmaceutically acceptable salt, tautomer, stereoisomer, hydrate, or isotope thereof, wherein R₁, R₅, Rₓ, and R⁷ are defined as provided for Formula (I), m is an integer between 0 and 12; and n is an integer between 0 and 4.

In another embodiment, the compound of Formula (I) is a compound of Formula (I-g):

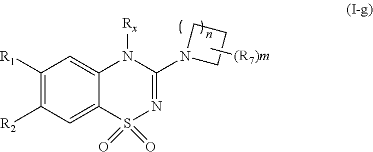

or a pharmaceutically acceptable salt, tautomer, stereoisomer, hydrate, or isotope thereof, wherein R₁, R₂, R₅, Rₓ, and R⁷ are defined as provided for Formula (I), m is an integer between 0 and 12; and n is an integer between 0 and 4.

In another embodiment, the compound of Formula (I) is a compound of Formula (I-h):

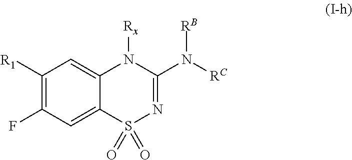

or a pharmaceutically acceptable salt, tautomer, stereoisomer, hydrate, or isotope thereof, wherein R₁, R₅, Rₓ, R^{B}, R^{C}, and R⁷ are defined as provided for Formula (I).

In another embodiment, the compound of Formula (I) is a compound of Formula (I-d):

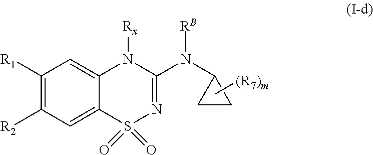

or a pharmaceutically acceptable salt, tautomer, stereoisomer, hydrate, or isotope thereof, wherein R₁, R₂, R₅, R^{B}, Rₓ, and R⁷ are defined as provided for Formula (I), m is an integer between 0 and 12; and n is an integer between 0 and 4.

In an embodiment, the compounds of Formula (I) described herein exhibit improved properties over the compounds in the art. The known compounds cause serious side effects due to lack of channel specificity. Moreover, the benefit for the patient group at large of compounds already known in the prior art is modest as the responsiveness of patients suffering from HI, in particularly CHI varies considerably within patients. It is assumed that this is due to their low specificity. In view of the high specificity of the compounds of the present invention responsiveness of patients suffering from HI, in particular CHI will be considerably better.

With respect to the compounds according to the present invention it is noted that they include all their acid addition and base salts, tautomeric forms, metabolites or prodrugs. The present invention further includes unsolvated forms, solvated forms including hydrated forms of the compounds of the present invention.

Preferred compounds according to the present invention are wherein said compounds are according to general formula (I) such that:
A is carbon (C);
B and D are Nitrogen (N); and
wherein A and B or A and D are connected via a double or single bond; and
wherein R₁ and R₂ are independently selected from the group consisting of hydrogen, fluorine, chlorine, bromine and iodine; and
wherein A and B form a double bond and Rₓ is H or wherein A and D form a double bond and R_{y} is H; and
wherein R₃ is independently selected from the group consisting of:

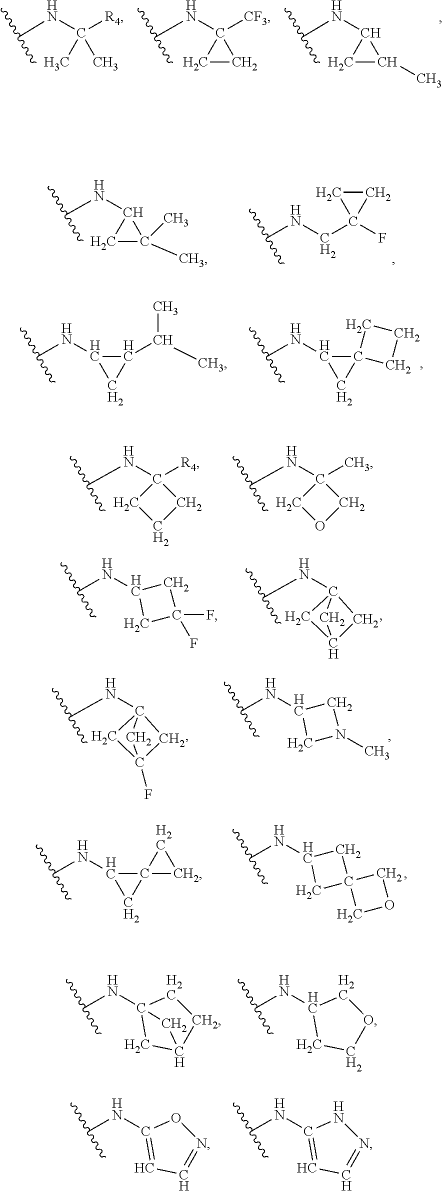

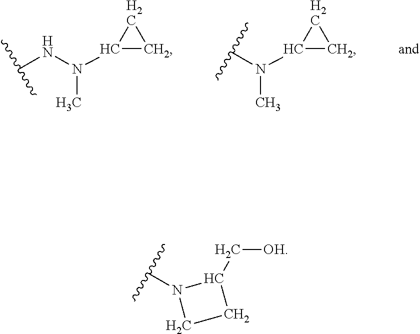

A second aspect of the present invention relates to a process for preparing the compounds according to the present invention. Said process comprises the steps of:
a) providing a first compound according to formula (X)

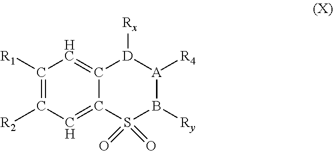

wherein:
   A is carbon (C);
   B and D are Nitrogen (N); and
   wherein A and B or A and D are connected via a double or single bond; and
   wherein R₁ and R₂ are independently selected from the group consisting of hydrogen, fluorine, chlorine, bromine and iodine; and
   wherein A and B form a double bond, Rₓ is H or wherein A and D form a double bond, R_{y} is H and
   wherein R₄ is independently selected from the group consisting of fluorine, chlorine, bromine and iodine, preferably chlorine:
b) providing a second compound according to formula H—R₃, wherein R₃ is selected from the group consisting of:

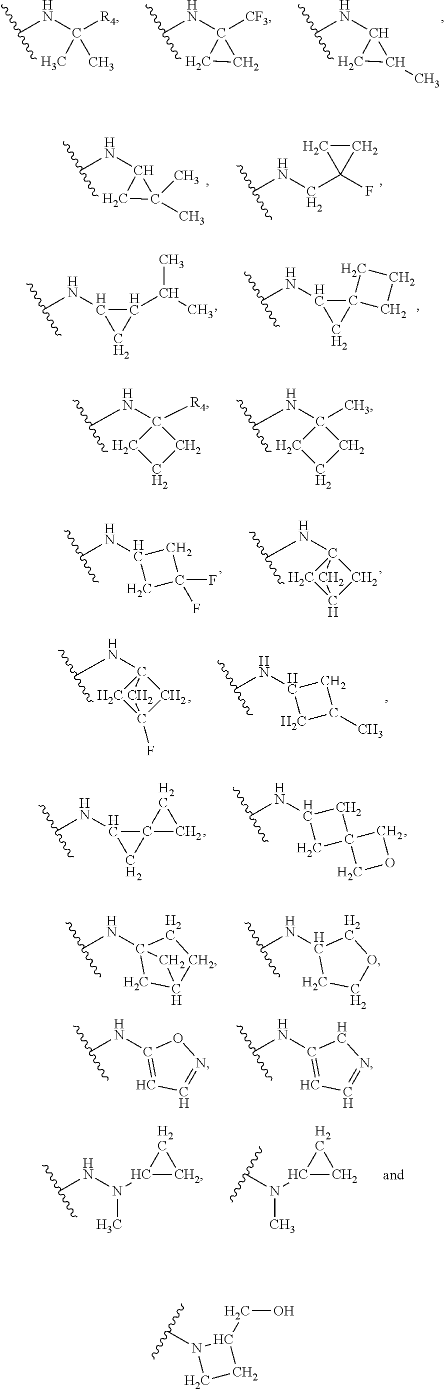

and wherein R₄ is selected from the group consisting of C1-C3 alkyl optionally substituted with one to two substituents selected from the group consisting of fluoro and hydroxy, ethynyl, C1-C2 alkoxy, (methoxy)-C1-C2 alkyl, cyano, fluoro, (methylsulfonyl)-C1-C2 alkyl, (dimethylamino)-C1-C2 alkyl and n-methyl carbamoyl.
c) reacting said first compound with said second compound under conditions that result in the formation of a compound of the invention.

In embodiments the reaction is performed in a solvent comprising a polar aprotic solvent, such as dichloromethane, tetrahydrofuran or dioxane, preferably dioxane.

In preferred embodiments the reaction is performed at a temperature between 20° C. and 200° C., preferably at a temperature between 30° C. and 160° C.

In preferred embodiments the reaction is performed in the presence of a base, preferably a sterically hindered base, more preferably a sterically hindered amine base, such as triethylamine or N,N-diisopropylethylamine, preferably N,N-diisopropylethylamine.

A third aspect of the present invention relates to a pharmaceutical composition comprising a compound according to the present invention and a pharmaceutically acceptable carrier. The pharmaceutical composition according to the present invention comprises besides a compound according the present invention also a pharmaceutically acceptable carrier and/or excipient.

Examples of categories of excipients include, but are not limited to, binders, disintegrants, lubricants, glidants, fillers and diluents. One of ordinary skill in the art may select one or more of the aforementioned excipients with respect to the particular desired properties of the granulate and/or solid oral dosage form by routine experimentation and without any undue burden. The amount of each excipient used may vary within ranges conventional in the art.

The following references which are all hereby incorporated by reference disclose techniques and excipients used to formulate oral dosage forms. See “The Handbook of Pharmaceutical Excipients”, 4th edition, Rowe et al., Eds., American Pharmaceuticals Association (2003); and “Remington: The Science and Practice of Pharmacy”, 20th edition, Gennaro, Ed., Lippincott Williams & Wilkins (2000).

A last aspect of the present invention relates to the use of the compounds or pharmaceutical composition according to the present invention in the treatment of subjects, in particularly human subjects suffering from hyperinsulinism (HI), more particularly congenital hyperinsulinism (CHI).

In an embodiment, the compound of the present disclosure is a compound provided in Table 1.

TABLE 1
Exemplary compounds of Formula (I).

| Compound No. | Structure |
|---|---|
| 1 | 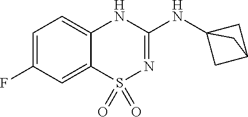 [SMILES: O=S1(=O)N=C(NC23CC(C2)C3)Nc2ccc(F)cc21] |
| 2 | 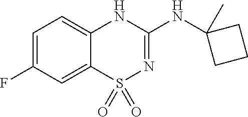 [SMILES: CC1(NC2=NS(=O)(=O)c3cc(F)ccc3N2)CCC1] |
| 3 | 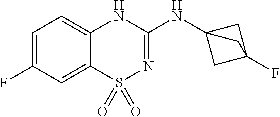 [SMILES: O=S1(=O)N=C(NC23CC(F)(C2)C3)Nc2ccc(F)cc21] |
| 4 | 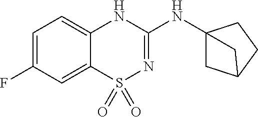 [SMILES: O=S1(=O)N=C(NC23CCC(C2)C3)Nc2ccc(F)cc21] |
| 5 | 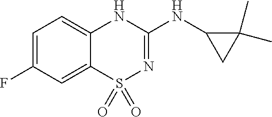 [SMILES: CC1(C)CC1NC1=NS(=O)(=O)c2cc(F)ccc2N1] |
| 6 | 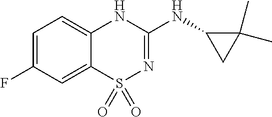 [SMILES: CC1(C)C[C@@H]1NC1=NS(=O)(=O)c2cc(F)ccc2N1] |
| 7 | 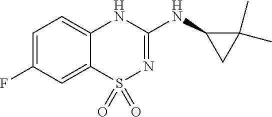 [SMILES: CC1(C)C[C@H]1NC1=NS(=O)(=O)c2cc(F)ccc2N1] |
| 8 | 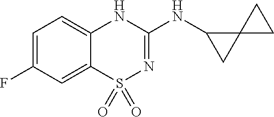 [SMILES: O=S1(=O)N=C(NC2CC23CC3)Nc2ccc(F)cc21] |
| 9 | 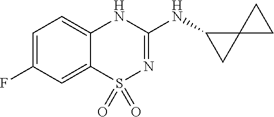 [SMILES: O=S1(=O)N=C(N[C@H]2CC23CC3)Nc2ccc(F)cc21] |
| 10 | 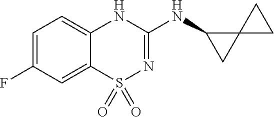 [SMILES: O=S1(=O)N=C(N[C@@H]2CC23CC3)Nc2ccc(F)cc21] |
| 11 | 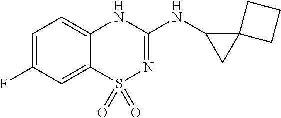 [SMILES: O=S1(=O)N=C(NC2CC23CCC3)Nc2ccc(F)cc21] |
| 12 | 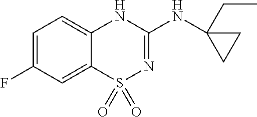 [SMILES: CCC1(NC2=NS(=O)(=O)c3cc(F)ccc3N2)CC1] |
| 13 | 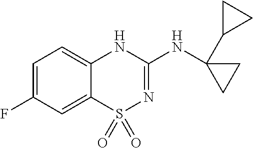 [SMILES: O=S1(=O)N=C(NC2(C3CC3)CC2)Nc2ccc(F)cc21] |
| 14 | 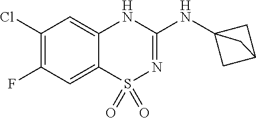 [SMILES: O=S1(=O)N=C(NC23CC(C2)C3)Nc2cc(Cl)c(F)cc21] |
| 15 | 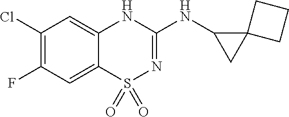 [SMILES: O=S1(=O)N=C(NC2CC23CCC3)Nc2cc(Cl)c(F)cc21] |
| 16 | 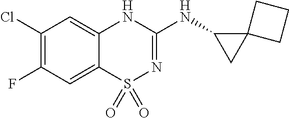 [SMILES: O=S1(=O)N=C(N[C@H]2CC23CCC3)Nc2cc(Cl)c(F)cc21] |
| 17 | 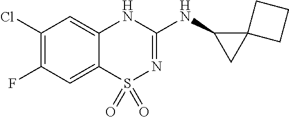 [SMILES: O=S1(=O)N=C(N[C@@H]2CC23CCC3)Nc2cc(Cl)c(F)cc21] |
| 18 | 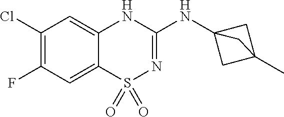 [SMILES: CC12CC(NC3=NS(=O)(=O)c4cc(F)c(Cl)cc4N3)(C1)C2] |
| 19 | 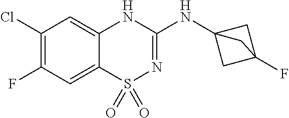 [SMILES: O=S1(=O)N=C(NC23CC(F)(C2)C3)Nc2cc(Cl)c(F)cc21] |
| 20 | 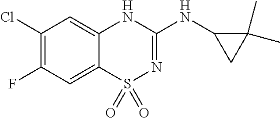 [SMILES: CC1(C)CC1NC1=NS(=O)(=O)c2cc(F)c(Cl)cc2N1] |
| 21 | 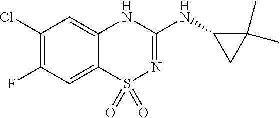 [SMILES: CC1(C)C[C@@H]1NC1=NS(=O)(=O)c2cc(F)c(Cl)cc2N1] |
| 22 | 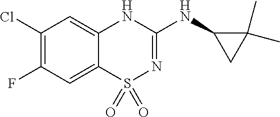 [SMILES: CC1(C)C[C@H]1NC1=NS(=O)(=O)c2cc(F)c(Cl)cc2N1] |
| 23 | 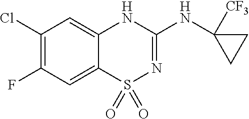 [SMILES: O=S1(=O)N=C(NC2(C(F)(F)F)CC2)Nc2cc(Cl)c(F)cc21] |
| 24 | 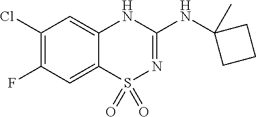 [SMILES: CC1(NC2=NS(=O)(=O)c3cc(F)c(Cl)cc3N2)CCC1] |
| 25 | 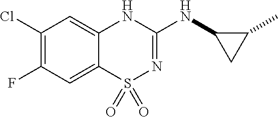 |
| 26 | 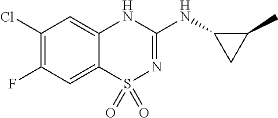 [SMILES: C[C@H]1C[C@@H]1NC1=NS(=O)(=O)c2cc(F)c(Cl)cc2N1] |
| 27 | 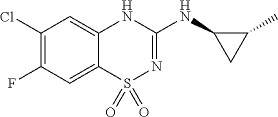 [SMILES: C[C@@H]1C[C@H]1NC1=NS(=O)(=O)c2cc(F)c(Cl)cc2N1] |
| 28 | 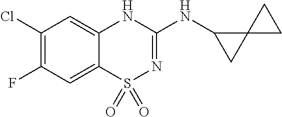 [SMILES: O=S1(=O)N=C(NC2CC23CC3)Nc2cc(Cl)c(F)cc21] |
| 29 | 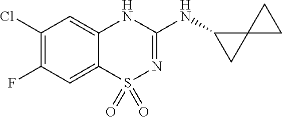 [SMILES: O=S1(=O)N=C(N[C@H]2CC23CC3)Nc2cc(Cl)c(F)cc21] |
| 30 | 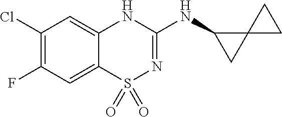 [SMILES: O=S1(=O)N=C(N[C@@H]2CC23CC3)Nc2cc(Cl)c(F)cc21] |
| 31 | 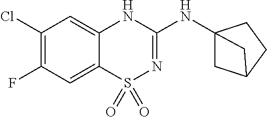 [SMILES: O=S1(=O)N=C(NC23CCC(C2)C3)Nc2cc(Cl)c(F)cc21] |
| 32 | 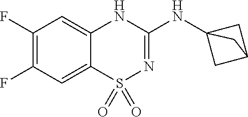 [SMILES: O=S1(=O)N=C(NC23CC(C2)C3)Nc2cc(F)c(F)cc21] |
| 33 | 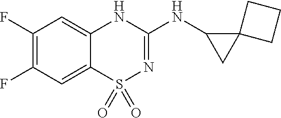 [SMILES: O=S1(=O)N=C(NC2CC23CCC3)Nc2cc(F)c(F)cc21] |
| 34 | 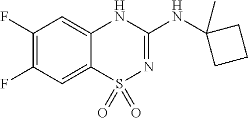 [SMILES: CC1(NC2=NS(=O)(=O)c3cc(F)c(F)cc3N2)CCC1] |
| 35 | 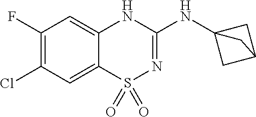 [SMILES: O=S1(=O)N=C(NC23CC(C2)C3)Nc2cc(F)c(Cl)cc21] |
| 36 | 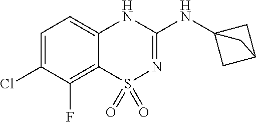 [SMILES: O=S1(=O)N=C(NC23CC(C2)C3)Nc2ccc(Cl)c(F)c21] |
| 37 | 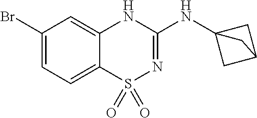 [SMILES: O=S1(=O)N=C(NC23CC(C2)C3)Nc2cc(Br)ccc21] |
| 38 | 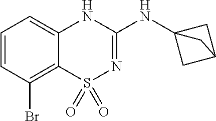 [SMILES: O=S1(=O)N=C(NC23CC(C2)C3)Nc2cccc(Br)c21] |
| 39 | 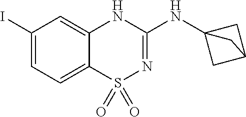 [SMILES: O=S1(=O)N=C(NC23CC(C2)C3)Nc2cc(I)ccc21] |
| 40 | 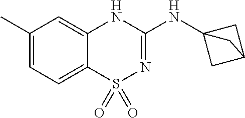 [SMILES: Cc1ccc2c(c1)NC(NC13CC(C1)C3)=NS2(=O)=O] |
| 41 | 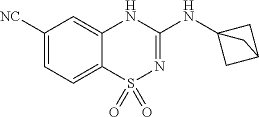 [SMILES: N#Cc1ccc2c(c1)NC(NC13CC(C1)C3)=NS2(=O)=O] |
| 42 | 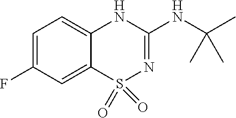 [SMILES: CC(C)(C)NC1=NS(=O)(=O)c2cc(F)ccc2N1] |
| 43 | 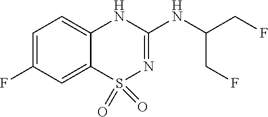 [SMILES: O=S1(=O)N=C(NC(CF)CF)Nc2ccc(F)cc21] |
| 44 | 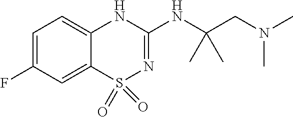 [SMILES: CN(C)CC(C)(C)NC1=NS(=O)(=O)c2cc(F)ccc2N1] |
| 45 | 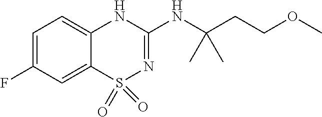 [SMILES: COCCC(C)(C)NC1=NS(=O)(=O)c2cc(F)ccc2N1] |
| 46 | 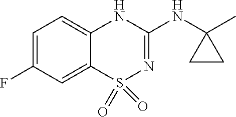 [SMILES: CC1(NC2=NS(=O)(=O)c3cc(F)ccc3N2)CC1] |
| 47 | 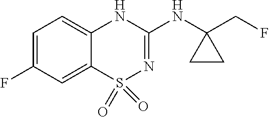 [SMILES: O=S1(=O)N=C(NC2(CF)CC2)Nc2ccc(F)cc21] |
| 48 | 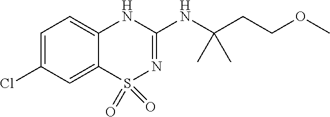 [SMILES: COCCC(C)(C)NC1=NS(=O)(=O)c2cc(Cl)ccc2N1] |
| 49 | 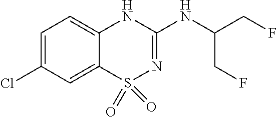 [SMILES: O=S1(=O)N=C(NC(CF)CF)Nc2ccc(Cl)cc21] |
| 50 | 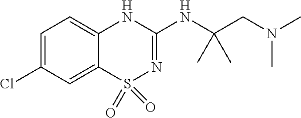 [SMILES: CN(C)CC(C)(C)NC1=NS(=O)(=O)c2cc(Cl)ccc2N1] |
| 51 | 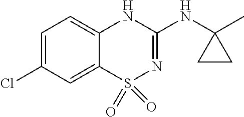 [SMILES: CC1(NC2=NS(=O)(=O)c3cc(Cl)ccc3N2)CC1] |
| 52 | 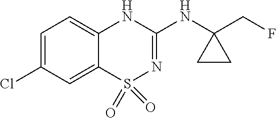 [SMILES: O=S1(=O)N=C(NC2(CF)CC2)Nc2ccc(Cl)cc21] |
| 53 | 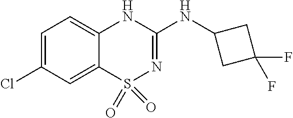 [SMILES: O=S1(=O)N=C(NC2CC(F)(F)C2)Nc2ccc(Cl)cc21] |
| 54 | 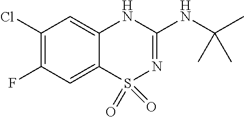 [SMILES: CC(C)(C)NC1=NS(=O)(=O)c2cc(F)c(Cl)cc2N1] |
| 55 | 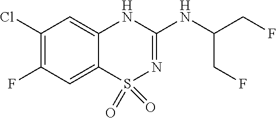 [SMILES: O=S1(=O)N=C(NC(CF)CF)Nc2cc(Cl)c(F)cc21] |
| 56 | 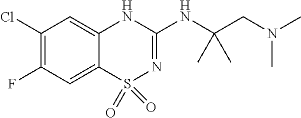 [SMILES: CN(C)CC(C)(C)NC1=NS(=O)(=O)c2cc(F)c(Cl)cc2N1] |
| 57 | 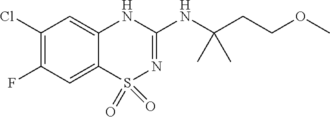 [SMILES: COCCC(C)(C)NC1=NS(=O)(=O)c2cc(F)c(Cl)cc2N1] |
| 58 | 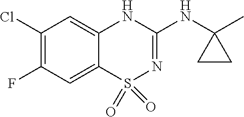 [SMILES: CC1(NC2=NS(=O)(=O)c3cc(F)c(Cl)cc3N2)CC1] |
| 59 | 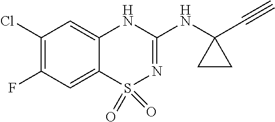 [SMILES: C#CC1(NC2=NS(=O)(=O)c3cc(F)c(Cl)cc3N2)CC1] |
| 60 | 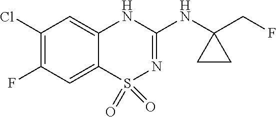 [SMILES: O=S1(=O)N=C(NC2(CF)CC2)Nc2cc(Cl)c(F)cc21] |
| 61 | 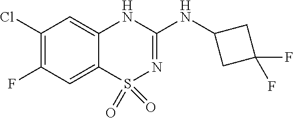 [SMILES: O=S1(=O)N=C(NC2CC(F)(F)C2)Nc2cc(Cl)c(F)cc21] |
| 62 | 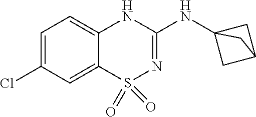 [SMILES: O=S1(=O)N=C(NC23CC(C2)C3)Nc2ccc(Cl)cc21] |
| 63 | 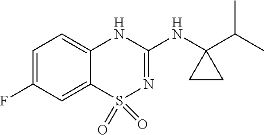 [SMILES: CC(C)C1(NC2=NS(=O)(=O)c3cc(F)ccc3N2)CC1] |
| 64 | 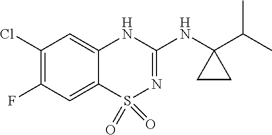 [SMILES: CC(C)C1(NC2=NS(=O)(=O)c3cc(F)c(Cl)cc3N2)CC1] |
| 65 | 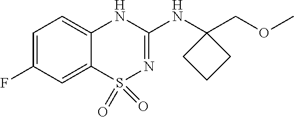 [SMILES: COCC1(NC2=NS(=O)(=O)c3cc(F)ccc3N2)CCC1] |
| 66 | 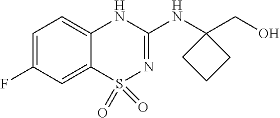 [SMILES: O=S1(=O)N=C(NC2(CO)CCC2)Nc2ccc(F)cc21] |
| 67 | 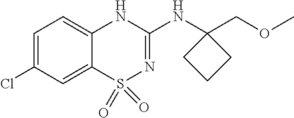 [SMILES: COCC1(NC2=NS(=O)(=O)c3cc(Cl)ccc3N2)CCC1] |
| 68 | 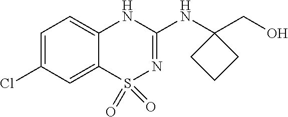 [SMILES: O=S1(=O)N=C(NC2(CO)CCC2)Nc2ccc(Cl)cc21] |
| 69 | 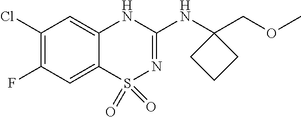 [SMILES: COCC1(NC2=NS(=O)(=O)c3cc(F)c(Cl)cc3N2)CCC1] |
| 70 | 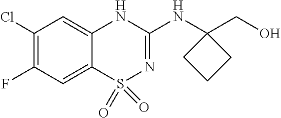 [SMILES: O=S1(=O)N=C(NC2(CO)CCC2)Nc2cc(Cl)c(F)cc21] |
| 71 | 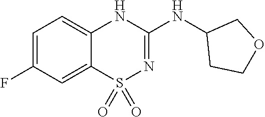 [SMILES: O=S1(=O)N=C(NC2CCOC2)Nc2ccc(F)cc21] |
| 72 | 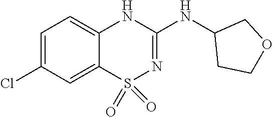 [SMILES: O=S1(=O)N=C(NC2CCOC2)Nc2ccc(Cl)cc21] |
| 73 | 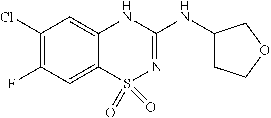 [SMILES: O=S1(=O)N=C(NC2CCOC2)Nc2cc(Cl)c(F)cc21] |
| 74 | 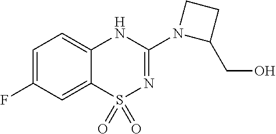 [SMILES: O=S1(=O)N=C(N2CCC2CO)Nc2ccc(F)cc21] |
| 75 | 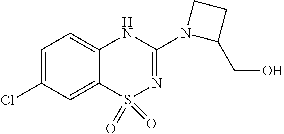 [SMILES: O=S1(=O)N=C(N2CCC2CO)Nc2ccc(Cl)cc21] |
| 76 | 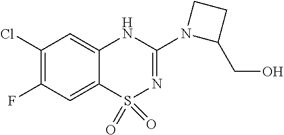 [SMILES: O=S1(=O)N=C(N2CCC2CO)Nc2cc(Cl)c(F)cc21] |

In an embodiment, the compound of Formula (I) is selected from:

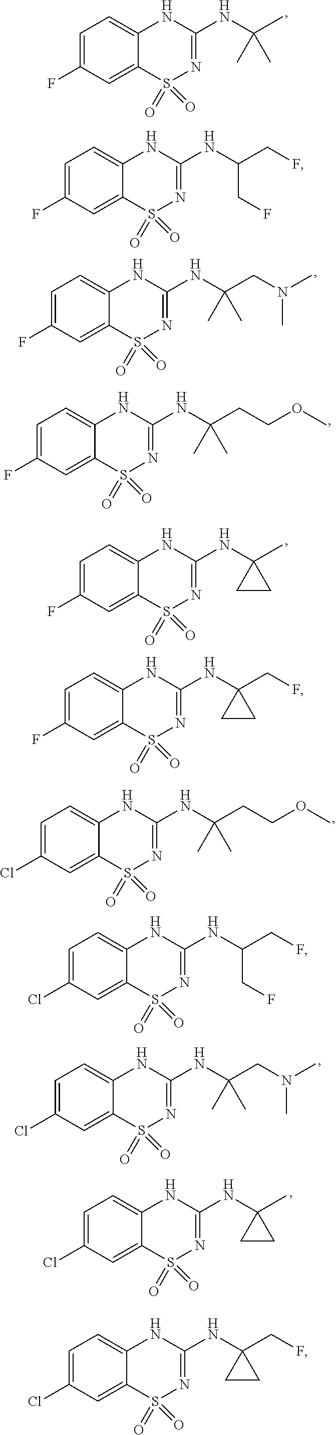

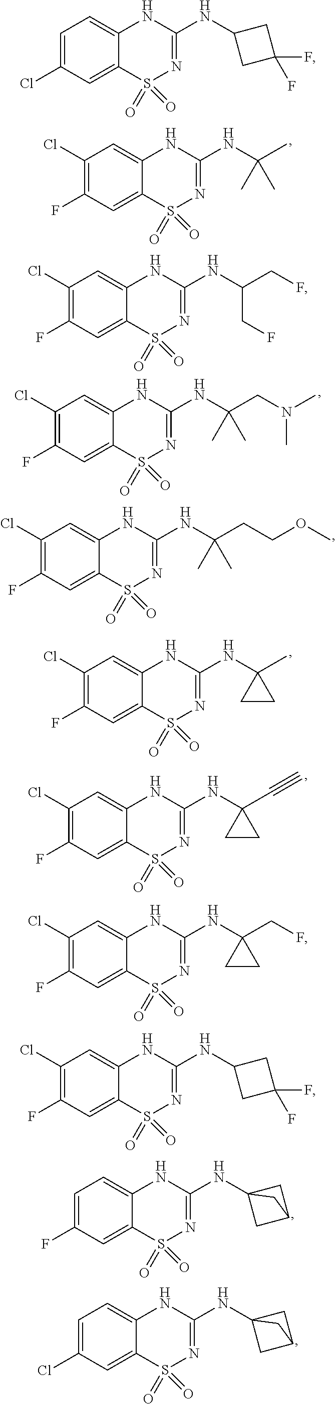

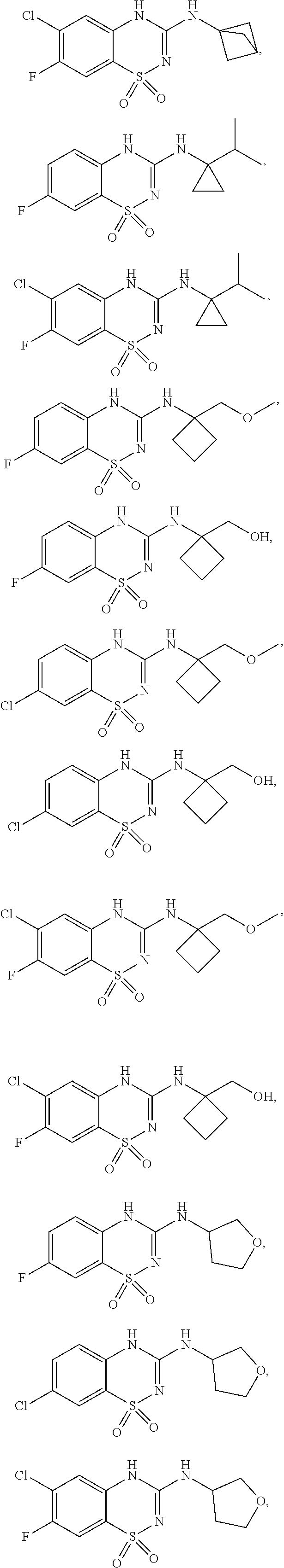

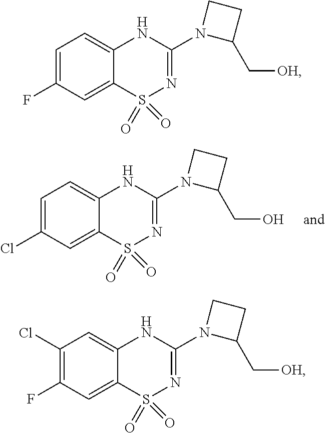

or a pharmaceutically acceptable salt thereof.

In preferred embodiments, the compound of Formula (I) is selected from

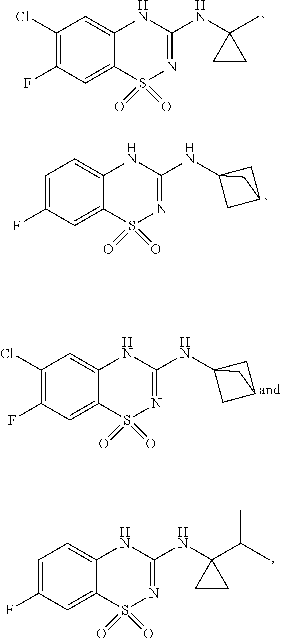 [SMILES: CC1(NC2=NS(=O)(=O)c3cc(F)c(Cl)cc3N2)CC1 ; O=S1(=O)N=C(NC23CC(C2)C3)Nc2ccc(F)cc21 ; O=S1(=O)N=C(NC23CC(C2)C3)Nc2cc(Cl)c(F)cc21 ; CC(C)C1(NC2=NS(=O)(=O)c3cc(F)ccc3N2)CC1]

or a pharmaceutically acceptable salt thereof.

In an embodiment, the compound of Formula (I) is Compound No. 1 or a pharmaceutically acceptable salt thereof. In In an embodiment, the compound of Formula (I) is Compound No. 2 or a pharmaceutically acceptable salt thereof. In an embodiment, the compound of Formula (I) is Compound No. 3 or a pharmaceutically acceptable salt thereof. In an embodiment, the compound of Formula (I) is Compound No. 4 or a pharmaceutically acceptable salt thereof. In an embodiment, the compound of Formula (I) is Compound No. 5 or a pharmaceutically acceptable salt thereof. In an embodiment, the compound of Formula (I) is Compound No. 6 or a pharmaceutically acceptable salt thereof. In an embodiment, the compound of Formula (I) is Compound No. 7 or a pharmaceutically acceptable salt thereof. In an embodiment, the compound of Formula (2) is Compound No. 8 or a pharmaceutically acceptable salt thereof. In an embodiment, the compound of Formula (I) is Compound No. 9 or a pharmaceutically acceptable salt thereof. In an embodiment, the compound of Formula (I) is Compound No. 10 or a pharmaceutically acceptable salt thereof. In an embodiment, the compound of Formula (I) is Compound No. 11 or a pharmaceutically acceptable salt thereof. In an embodiment, the compound of Formula (I) is Compound No. 12 or a pharmaceutically acceptable salt thereof. In an embodiment, the compound of Formula (I) is Compound No. 13 or a pharmaceutically acceptable salt thereof. In an embodiment, the compound of Formula (I) is Compound No. 14 or a pharmaceutically acceptable salt thereof. In an embodiment, the compound of Formula (I) is Compound No. 15 or a pharmaceutically acceptable salt thereof. In an embodiment, the compound of Formula (I) is Compound No. 16 or a pharmaceutically acceptable salt thereof. In an embodiment, the compound of Formula (I) is Compound No. 17 or a pharmaceutically acceptable salt thereof. In an embodiment, the compound of Formula (I) is Compound No. 18 or a pharmaceutically acceptable salt thereof. In an embodiment, the compound of Formula (I) is Compound No. 19 or a pharmaceutically acceptable salt thereof. In an embodiment, the compound of Formula (I) is Compound No. 20 or a pharmaceutically acceptable salt thereof. In an embodiment, the compound of Formula (I) is Compound No. 21 or a pharmaceutically acceptable salt thereof. In an embodiment, the compound of Formula (I) is Compound No. 22 or a pharmaceutically acceptable salt thereof. In an embodiment, the compound of Formula (I) is Compound No. 23 or a pharmaceutically acceptable salt thereof. In an embodiment, the compound of Formula (I) is Compound No. 24 or a pharmaceutically acceptable salt thereof. In an embodiment, the compound of Formula (I) is Compound No. 25 or a pharmaceutically acceptable salt thereof. In an embodiment, the compound of Formula (I) is Compound No. 26 or a pharmaceutically acceptable salt thereof. In an embodiment, the compound of Formula (I) is Compound No. 27 or a pharmaceutically acceptable salt thereof. In an embodiment, the compound of Formula (I) is Compound No. 28 or a pharmaceutically acceptable salt thereof. In an embodiment, the compound of Formula (I) is Compound No. 29 or a pharmaceutically acceptable salt thereof. In an embodiment, the compound of Formula (I) is Compound No. 30 or a pharmaceutically acceptable salt thereof. In an embodiment, the compound of Formula (I) is Compound No. 31 or a pharmaceutically acceptable salt thereof. In an embodiment, the compound of Formula (I) is Compound No. 32 or a pharmaceutically acceptable salt thereof. In an embodiment, the compound of Formula (I) is Compound No. 33 or a pharmaceutically acceptable salt thereof. In an embodiment, the compound of Formula (I) is Compound No. 34 or a pharmaceutically acceptable salt thereof. In an embodiment, the compound of Formula (I) is Compound No. 35 or a pharmaceutically acceptable salt thereof. In an embodiment, the compound of Formula (I) is Compound No. 36 or a pharmaceutically acceptable salt thereof. In an embodiment, the compound of Formula (I) is Compound No. 37 or a pharmaceutically acceptable salt thereof. In an embodiment, the compound of Formula (I) is Compound No. 38 or a pharmaceutically acceptable salt thereof. In an embodiment, the compound of Formula (I) is Compound No. 39 or a pharmaceutically acceptable salt thereof. In an embodiment, the compound of Formula (I) is Compound No. 40 or a pharmaceutically acceptable salt thereof. In an embodiment, the compound of Formula (I) is Compound No. 41 or a pharmaceutically acceptable salt thereof. In an embodiment, the compound of Formula (I) is Compound No. 42 or a pharmaceutically acceptable salt thereof. In an embodiment, the compound of Formula (I) is Compound No. 43 or a pharmaceutically acceptable salt thereof. In an embodiment, the compound of Formula (I) is Compound No. 44 or a pharmaceutically acceptable salt thereof. In an embodiment, the compound of Formula (I) is Compound No. 45 or a pharmaceutically acceptable salt thereof. In an embodiment, the compound of Formula (I) is Compound No. 46 or a pharmaceutically acceptable salt thereof. In an embodiment, the compound of Formula (I) is Compound No. 47 or a pharmaceutically acceptable salt thereof. v In an embodiment, the compound of Formula (I) is Compound No. 48 or a pharmaceutically acceptable salt thereof. In an embodiment, the compound of Formula (I) is Compound No. 49 or a pharmaceutically acceptable salt thereof. In an embodiment, the compound of Formula (I) is Compound No. 50 or a pharmaceutically acceptable salt thereof. In an embodiment, the compound of Formula (I) is Compound No. 51 or a pharmaceutically acceptable salt thereof. In an embodiment, the compound of Formula (I) is Compound No. 52 or a pharmaceutically acceptable salt thereof. In an embodiment, the compound of Formula (I) is Compound No. 53 or a pharmaceutically acceptable salt thereof. In an embodiment, the compound of Formula (I) is Compound No. 54 or a pharmaceutically acceptable salt thereof. In an embodiment, the compound of Formula (I) is Compound No. 55 or a pharmaceutically acceptable salt thereof. In an embodiment, the compound of Formula (I) is Compound No. 56 or a pharmaceutically acceptable salt thereof. In an embodiment, the compound of Formula (I) is Compound No. 57 or a pharmaceutically acceptable salt thereof. In an embodiment, the compound of Formula (I) is Compound No. 58 or a pharmaceutically acceptable salt thereof. In an embodiment, the compound of Formula (I) is Compound No. 59 or a pharmaceutically acceptable salt thereof. In an embodiment, the compound of Formula (I) is Compound No. 60 or a pharmaceutically acceptable salt thereof. In an embodiment, the compound of Formula (I) is Compound No. 61 or a pharmaceutically acceptable salt thereof. In an embodiment, the compound of Formula (I) is Compound No. 62 or a pharmaceutically acceptable salt thereof. In an embodiment, the compound of Formula (I) is Compound No. 63 or a pharmaceutically acceptable salt thereof. In an embodiment, the compound of Formula (I) is Compound No. 64 or a pharmaceutically acceptable salt thereof. In an embodiment, the compound of Formula (I) is Compound No. 65 or a pharmaceutically acceptable salt thereof. In an embodiment, the compound of Formula (I) is Compound No. 66 or a pharmaceutically acceptable salt thereof. In an embodiment, the compound of Formula (I) is Compound No. 67 or a pharmaceutically acceptable salt thereof. In an embodiment, the compound of Formula (I) is Compound No. 68 or a pharmaceutically acceptable salt thereof. In an embodiment, the compound of Formula (I) is Compound No. 69 or a pharmaceutically acceptable salt thereof. In an embodiment, the compound of Formula (I) is Compound No. 70 or a pharmaceutically acceptable salt thereof. In an embodiment, the compound of Formula (I) is Compound No. 71 or a pharmaceutically acceptable salt thereof. In an embodiment, the compound of Formula (I) is Compound No. 72 or a pharmaceutically acceptable salt thereof. In an embodiment, the compound of Formula (I) is Compound No. 73 or a pharmaceutically acceptable salt thereof. In an embodiment, the compound of Formula (I) is Compound No. 74 or a pharmaceutically acceptable salt thereof. In an embodiment, the compound of Formula (I) is Compound No. 75 or a pharmaceutically acceptable salt thereof. In an embodiment, the compound of Formula (I) is Compound No. 76 or a pharmaceutically acceptable salt thereof.

In some embodiments, the compound of Formula (I) provided herein is not a compound disclosed in WO 97/49692A; De Tullio P. et al. J Med Chem., 2003, 46:1, pp. 3342-3353. In some embodiments, the compounds provided herein do not include any of the compounds disclosed in Sharma B. K. et al., Int J Chem Sci., 2009, 7:2, pp. 655-671. In some embodiments, the compounds provided herein do not include any of the compounds disclosed in Escudero, J. et al., ACS Catalysis. 2022, 12, pp. 6857-6873. In some embodiments, the compounds provided herein do not include any of the compounds disclosed in Vlaar, T. et al., Angew Chem Int Ed., 2012, 51, pp. 13058-13061. In some embodiments, the compounds provided herein do not include any of the compounds disclosed in De Tullio, P. et al., J Med Chem., 2005, 48, pp. 4990-5000.

In an embodiment, the compound is not a compound disclosed in WO1997049692A1, WO2002074945A1, WO2003087089A1, WO2003091245A1, WO2005063742A2, WO2006045799A2, WO2007125048A1, WO2010093243A1, WO2013130411A1, WO2014046172A1, WO2019084271A1, WO2021236818A1, U.S. Pat. No. 4,035,374A, US20070254862A1, DE2757922A1, DE2757999A1, EP105732A2, EP112142A2, EP172968A1, EP355612A2, JP6284823B2, JP51054576A, or JP60072868A.

In an embodiment, the compound is not a compound disclosed in WO2003087089A1, WO2003091245A1, WO2004087053A2, WO2005058348A1, WO2005063742A2, WO2007053514A2, WO2007081521A2, WO2007136125A1, WO2009006483A1, WO2017098421A1, WO2019084271A1, WO2021236818A1, WO1997049692A1, U.S. Pat. No. 4,035,374A, EP105732A2, EP112142A2, EP355612A2, EP386931A1, JP2015214525A, JP2016011275A, JP3254698A, or JP60112781A.

In an embodiment, the compound is not a compound disclosed in WO2006025857A2, WO2006069806A1, WO2000037474A1, WO2001002410A1, WO2002000222A1, WO2002000665A1, WO2002050085A1, WO2003045954A1, WO2003045955A1, WO2003105896A1, WO2004005299A1, WO2005013962A1, WO2006045799A2, WO2006088798A2, WO2007020286A2, WO2007125048A1, WO2009000038A1, WO2022125784A1, WO1997026265A1, DK200400395A, or US20070254862A1.

In an embodiment, the compound is not a compound disclosed in WO2000037474A1, WO2001002410A1, WO2002000222A1, WO2002050085A1, WO2003045954A1, WO2003045955A1, WO2003087089A1, WO2003091245A1, WO2003105896A1, WO1999003861A1, WO1999032494A1, or DK200400395A.

Definitions

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl. “Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “nitro” refers to a substitutent having two oxygen atoms bound to a nitrogen atom, e.g., —NO₂.

As used herein, “oxo” refers to a carbonyl, i.e., —C(O)—.

The symbol “ [symbol] ” as used herein in relation to a compound of Formula (I) refers to an attachment point to another moiety or functional group within the compound.

Alkyl, alkenyl, alkynyl, haloalkyl, heteroalkyl, cycloalkyl, and heterocyclyl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form cycloalkyl or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutical composition” as used herein has its conventional meaning and refers to a composition which is pharmaceutically acceptable.

The term “pharmaceutically acceptable” as used herein has its conventional meaning and refers to compounds, material, compositions and/or dosage forms, which are, within the scope of sound medical judgement suitable for contact with the tissues of mammals, especially humans, without excessive toxicity, irritation, allergic response and problem complications commensurate with a reasonable benefit/risk ratio.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro- forms of phenylnitromethane that are likewise formed by treatment with acid or base. Tautomeric forms may be relevant to the attainment of the optimal chemical reactivity and biological activity of a compound of interest.

A “metabolite” of a compound of the current invention is an active derivative of a compound according to the invention which is produced when the compound is metabolized.

A “prodrug” is a compound that either is converted into a compound disclosed in the present invention in vivo or has the same active metabolite as a compound disclosed in this application.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “disease,” and “disorder” are used interchangeably.

An “effective amount” of a compound of Formula (I) refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

“Prevention,” “prevent,” and “preventing” as used herein refers to a treatment that comprises administering a therapy, e.g., administering a compound described herein (e.g., a compound of Formula (I)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease or disorder have developed or have been observed. In other embodiments, treatment may be administered in the absence of signs or symptoms of the disease, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The term “carrier” as used herein has its conventional meaning and refers to a pharmaceutically acceptable diluent, adjuvant, excipient or vehicle with which a pharmaceutically active ingredient is administered.

The term “excipient” as used herein has its conventional meaning and refers to a pharmaceutically acceptable ingredient, which is commonly used in the pharmaceutical technology for preparing a granulate, solid or liquid formulation.

The term “persistent or recurrent congenital hyperinsulinism (CHI)” comprises forms of hyperinsulinism (HI) which can be formed in neonates and children due to any genetic mutations and which is persistent despite continuous glucose administration or due to any other metabolic disorder known or unknown to the skilled person.

The term “transient CHI” comprises forms of HI which can be formed during fetal, neonatal and early childhood stages due to gene mutations and/or perinatal stress, such as prematurity, intrauterine growth retardation, small for gestational age, or perinatal asphyxia or due to any other metabolic disorder known or unknown to the skilled person.

### Methods of Use

Disclosed herein are methods for treating a disease or disorder with a compound, e.g., a compound of Formula (I) or a pharmaceutically acceptable salt thereof. Exemplary diseases or disorders may include a metabolic disorder (e.g., CHI), cancer, a neurological disorder, a cardiovascular disorder, a pulmonary disorder, an integumentary disorder, a sexual disorder, a urinary disorder, or a symptom thereof.

In some embodiments, the disease or disorder is a metabolic disorder. In some embodiments, the metabolic disorder is hyperinsulinism (HI), congenital hyperinsulinism (CHI), persistent hyperinsulinism, or transient hyperinsulinism. In some embodiments, the metabolic disorder is a hyperinsulism-related syndrome, e.g., Beckwith-Wiedemann syndrome, hyperinsulinism-hyperammonaemia (HIHA) syndrome, Sotos syndrome, Turner syndrome, Costello syndrome, Kabuki syndrome, and the like. Glucose is an important source of energy for mammals, as it is transported from the intestines or liver to body cells via the bloodstream and is made available for cellular metabolism by secretion of the hormone insulin. By means of a delicate mechanism, referred to as glucose homeostasis, the body in healthy human subjects is able to maintain the glucose concentration within a preferred range of around 3.5 to 5.5 mmol/L. Blood glucose levels outside this normal range may be an indicator of a medical condition.

The major hormones involved in regulation of glucose metabolism over the time scale of hours are insulin and glucagon. Insulin and glucagon are secreted by the pancreatic islets and are both secreted in response to blood glucose levels, but in opposite fashion.

Insulin is an important peptide hormone involved in nutrient homeostasis which is produced by the β-cells in the pancreatic islets. It regulates the metabolism of carbohydrates and fats by promoting the transport of glucose from the blood to skeletal muscles and fat tissue. The general stimulus for insulin secretion is a high blood glucose. Although there is always a low level of insulin secreted by the pancreas, the amount secreted into the blood increases as the blood glucose rises.

On the other hand, when glucose levels are low, glucagon secretion increases and insulin secretion decreases. The most important effect of the glucagon increase is that it triggers the liver to release the glucose it has stored in its cells (as the polymer glycogen) into the bloodstream. The net effect of this release of glucose is the raising of the blood glucose concentration. Moreover, glucagon also induces the liver and other cells to make glucose out of building blocks obtained from other nutrients found in the body (e.g., protein).

Blood glucose levels outside the normal range may be an indicator of a medical condition. A persistently high level is referred to as hyperglycaemia and low levels are referred to as hypoglycaemia. One particular condition associated with a dysfunctional glucose homeostasis is hyperinsulinism.

Hyperinsulinism (HI) is defined as the elevated insulin levels in blood due to dysregulated release of the insulin from p-cells and causes hypoglycaemia. It can be genetic or acquired and transient or permanent. Hyperinsulinaemic hypoglycaemia (HH) is described as a clinically, genetically, and morphologically heterozygous conditions where the insulin secretion is persistent despite low blood glucose levels. The most severe and permanent forms of HH is due to congenital hyperinsulinism.

Congenital hyperinsulinism (CHI) is an inherited form of hyperinsulinaemic hypoglycaemia (HH) due to mutations in genes involved in the regulation of insulin secretion. CHI is a permanent form of hypoglycaemia in neonates, infants and children which causes irreversible brain damage due to the secondary metabolic actions of insulin. The excessive release of insulin causes suppression of lipolysis and reduction in ketones which are alternative energy sources for the brain. The rise of the insulin levels also inhibits secretion of glucagon which hinders the fat breakdown causing again direct damage of the brain. Therefore, up to 48% of children having recurrent hypoglycaemia suffer from brain damage.

The genetic basis of CHI has been related to 23 mutations in essential genes controlling insulin secretion and two of these genes, namely ABCC8 and KCNJ11, are genes encoding K_{ATP} channel proteins SUR1 and Kir6.2, regulating the insulin release from the pancreatic β-cell among other proteins.

In some embodiments, the metabolic disorder is obesity, e.g., hypothalamic obesity. In some embodiments, the metabolic disorder is diabetes, e.g., Type 1 or Type 2 diabetes, or first-phase diabetes or a pre-diabetes syndrome. In some embodiments, the metabolic disorder is a genetic or epigenetic disorder, e.g., Prader-Willi syndrome, Alstrom syndrome, Bardet-Biedl syndrome, or Smith-Magenis syndrome. In some embodiments, the metabolic disorder is Prader-Willi syndrome. In some embodiments, the metabolic disorder is Alstrom syndrome. In some embodiments, the metabolic disorder is Bardet-Biedl syndrome. In some embodiments, the metabolic disorder is Smith-Magenis syndrome. In an embodiment, the disease or disorder is ischemia.

In some embodiments, the disease or disorder is alopecia or baldness.

In some embodiments, the methods described herein directly or indirectly reduce or alleviate at least one symptom of a disease or disorder (e.g., a disease or disorder described herein).

In some embodiments, the methods described herein may treat or alleviate at least one symptom of the metabolic disorder, e.g., an increase in waist circumference of at least 2 cm relative to a reference (e.g., the waist circumference of a subject before onset of the metabolic disorder); an increase in blood pressure relative to a reference (e.g., the blood pressure of a subject before onset of the metabolic disorder); hyperglycemia or an increase in fasting blood sugar relative to a reference (e.g., the fasting blood sugar of a subject before the onset of the metabolic disorder); an increase in thirst (e.g., an increase in thirst relative to a reference, e.g., the level of thirst of a subject before the onset the metabolic disorder); an increase in fatigue (e.g., an increase in fatigue relative to a reference, e.g., the level of fatigue of a subject before the onset of a metabolic disorder); or an increase in urination (e.g., an increase in urination relative to a reference, e.g., the frequency or amount of urination of a subject before the onset of a metabolic disorder).

In some embodiments, the methods described herein may prevent or slow the onset of a disease or disorder, e.g., a metabolic disorder.

In some embodiments, the subject may have a comorbidity, e.g., obesity, hyperphagia or hyperphagia-related syndromes, unwanted appetite, hypoglycemia, hyperglycemia, hyperlipidemia, hypercholesterolemia, or hypertriglyceridemia. In some embodiments, the metabolic disorder is hyperinsulinemia, e.g., chronic hyperinsulinemia (CHI). In some embodiments, the metabolic disorder is pre-diabetes, type I diabetes, or type II diabetes. In some embodiments, the metabolic disorder is a diabetological condition.

In some embodiments, the subject may have had interventional surgery, e.g., bariatric surgery.

In some embodiments, the methods described herein may treat a metabolic disorder. In some embodiments, the methods described herein may provide a counterregulatory response to hypoglycemia in subject with diabetes, e.g., type I or type II diabetes. In some embodiments, the methods described herein may be useful in combination with a second agent, e.g., a sulfonylurea prior to a bolus to restore normal insulin levels in a subject, e.g., a subject with diabetes, e.g., type I or type II diabetes.

In some embodiments, the disease or disorder is a cancer. In some embodiments, the cancer is a gastrointestinal cancer, e.g., oesophageal cancer, gastric cancer, pancreatic cancer, liver cancer, gallbladder cancer, colorectal cancer, anal cancer, or a gastrointestinal carcinoid tumour. In some embodiments, the gastrointestinal cancer is oesophageal cancer. In some embodiments, the gastrointestinal cancer is gastric cancer. In some embodiments, the gastrointestinal cancer is pancreatic cancer. In some embodiments, the gastrointestinal cancer is a pancreatic cancer, wherein the pancreatic cancer is an insulinoma. In some embodiments, the gastrointestinal cancer is liver cancer. In some embodiments, the gastrointestinal cancer is gallbladder cancer. In some embodiments, the gastrointestinal cancer is colorectal cancer. In some embodiments, the gastrointestinal cancer is anal cancer. In some embodiments, the gastrointestinal cancer is a gastrointestinal cancer.

In some embodiments, the disease or disorder is a neurological disorder, e.g., Alzheimer's Disease, Parkinson's Disease, Multiple Sclerosis, migraine (e.g., chronic migraine), epilepsy and epilepsy-related syndromes.

In some embodiments, the methods as described herein directly or indirectly reduce or alleviate at least one symptom of a disease or disorder, e.g., a neurological disease or disorder described herein. In some embodiments, the methods directly or indirectly reduce or alleviate pain or aid in pain management, i.e., the methods as described herein directly or indirectly function in analgesia of a disease or a disorder, e.g., a neurological disorder.

In some embodiments, the methods as described herein directly or indirectly alleviate a symptom of a disease or disorder, e.g., a neurological disorder. In some embodiments, the methods as described herein provide neuroprotection in a subject in need thereof, e.g., in a subject with a neurological disorder, e.g., Alzheimer's Disease, Parkinson's Disease, or Multiple Sclerosis.

In some embodiments, the disease or disorder is a cardiovascular disorder, e.g., ischemia, ischemia-reperfusion injury, hypertension, coronary spasm, ocular pressure, peripheral vascular disease. In some embodiments, the cardiovascular disorder is angina, cardioplegia, a ventricular septal defect, atrial fibrillation, arrythmia, coronary artery disease, or myocardial stunning.

In some embodiments, the methods as described herein provide cardiooprotection in a subject in need thereof, e.g., in a subject with a cardiovascular disorder, e.g., angina, cardioplegia, a ventricular septal defect, atrial fibrillation, arrythmia, coronary artery disease, or myocardial stunning.

In some embodiments, the disease or disorder is a pulmonary disorder, e.g., pulmonary hypertension or asthma.

In some embodiments, the disease or disorder is an integumentary disorder, e.g., a disorder of the hair, skin, and nails, inter alia. In some embodiments, the disease or disorder is alopecia, baldness, e.g., male-pattern baldness, or disorders of hair follicular growth.

In some embodiments, the disease or disorder is a sexual disorder, e.g., male impotence.

In some embodiments, the disease or disorder is a urinary disorder, e.g., detrusor hyperreactivity.

In an embodiment, the subject is a mammal, e.g., a human. In an embodiment, the subject is an adult (e.g., over the age of 18 years old) or a child (e.g., under the age of 18, 12, 10, 8, 6, 4, or 2 years old). In an embodiment, the subject has been treated for a metabolic disorder previously. For example, the subject may be a bariatric surgery patient.

### Routes of Administration and Dosages

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention are administered orally or parenterally at dosage levels of about 0.01 mg/kg to about 100 mg/kg and preferably from about 1 mg/kg to about 50 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms optionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions are formulated according to the known art using suitable dispersing or wetting agents and suspending agents. The sterile injectable preparation are also a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This is accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also optionally comprises buffering agents.

Solid compositions of a similar type are also employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type are also employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also comprise, as is normal practice, additional substances other than inert diluents, e.g., tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### ENUMERATED EMBODIMENTS

1. A compound having the structure of Formula (I):

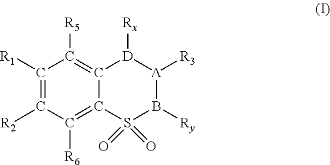

or a pharmaceutically acceptable salt, tautomer, stereoisomer, hydrate, or isotope thereof, wherein:
   A is C(R′);
   each of B and D are independently N; wherein A and B or A and D are connected via a double or single bond;
   each of R₁, R₂, R₅, and R₆ are independently hydrogen, C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano; wherein A and B form a double bond and Rₓ is hydrogen or wherein A and D form a double bond and R_{y} is hydrogen;
   R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), wherein alkyl, alkenyl, alkynyl, heteroalkyl, haloalkyl, cycloalkyl, and heterocyclyl are each optionally substituted with one or more R₄;
   R′ is absent, hydrogen, or C1-C6 alkyl;
   each R₄ is independently C1-C6 alkyl, C1-C6 heteroalkyl, C1-C6 haloalkyl, cycloalkyl, heterocyclyl, fluorine, chlorine, bromine, or iodine;
   R^{A} is hydrogen, C1-C6 alkyl, C2-C6 alkenyl, C1-C6 alkynyl, C1-C6 heteroalkyl, C1-C6 haloalkyl, cycloalkyl, or heterocyclyl, wherein alkyl, alkenyl, alkynyl, heteroalkyl, haloalkyl, cycloalkyl, and heterocyclyl are each optionally substituted with one or more R₇;
   each of R^{B} and R^{C} is independently hydrogen, C1-C6 alkyl, C2-C6 alkenyl, C1-C6 alkynyl, C1-C6 heteroalkyl, C1-C6 haloalkyl, cycloalkyl, heterocyclyl, wherein alkyl, alkenyl, alkynyl, heteroalkyl, haloalkyl, cycloalkyl, and heterocyclyl are each optionally substituted with one or more R₇; or
   wherein R^{B} and R^{C} are taken together to form heterocyclyl optionally substituted with one or more R⁷;
   each R⁷ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano.
2. The compound of embodiment 1, wherein each of R₁ and R₂ is independently hydrogen, C1-C6 alkyl, C1-C6 heteroalkyl, C1-C6 haloalkyl, —OR^{A}, fluorine, chlorine, bromine, iodine, nitro, or cyano.
3. The compound of any one of the preceding embodiments, wherein each of R₁ and R₂ is independently hydrogen, fluorine, chlorine, bromine, iodine, or cyano.
4. The compound of any one of the preceding embodiments, wherein each of R₁ and R₂ is independently hydrogen, fluorine, or chlorine.
5. The compound of any one of the preceding embodiments, wherein R₁ is hydrogen and R₂ is fluorine, chlorine, bromine, iodine, nitro, or cyano.
6. The compound of any one of the preceding embodiments, wherein R₂ is fluorine, chlorine, bromine, iodine, nitro, or cyano and R₂ is fluorine, chlorine, bromine, iodine, nitro, or cyano.
7. The compound of any one of the preceding embodiments, wherein R₁ is hydrogen and R₂ is fluorine.
8. The compound of any one of the preceding embodiments, wherein R₁ is hydrogen and R₂ is chlorine.
9. The compound of any one of the preceding embodiments, wherein R₁ is chlorine and R₂ is fluorine.
10. The compound of any one of the preceding embodiments, wherein each of R₁ and R₂ is independently fluorine.
11. The compound of any one of the preceding embodiments, wherein R₅ is H.
12. The compound of any one of the preceding embodiments, wherein R₆ is H.
13. The compound of any one of the preceding embodiments, wherein R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), each of which is optionally substituted with optionally substituted with one or more R₇.
14. The compound of any one of the preceding embodiments, wherein R₃ is C1-C6 heteroalkyl optionally substituted with one or more R₇.
15. The compound of any one of the preceding embodiments, wherein R₃ is C1-C6 haloalkyl, optionally substituted with one or more R₇.
16. The compound of any one of the preceding embodiments, wherein R₃ is cycloalkyl, optionally substituted with one or more R₇.
17. The compound of any one of the preceding embodiments, wherein R₃ is heterocyclyl, optionally substituted with one or more R₇.
18. The compound of any one of the preceding embodiments, wherein R₃ is N(R^{B})(R^{C}).
19. The compound of embodiment 18, wherein R^{B} and R^{C} are taken together to form heterocyclyl optionally substituted with one or more R⁷ (e.g., a 4-membered heterocyclyl, 5-membered heterocyclyl, 6-membered heterocyclyl, or a bridged heterocyclyl).
20. The compound of embodiment 18, wherein R^{B} is hydrogen.
21. The compound of any one of embodiments 18-20, wherein R^{C} is C1-C6 alkyl, C2-C6 alkenyl, C1-C6 alkynyl, C1-C6 heteroalkyl, C1-C6 haloalkyl, cycloalkyl, heterocyclyl, each optionally substituted with one or more R₇.
22. The compound of any one of embodiments 18-21, wherein R^{C} is C1-C6 alkyl optionally substituted with one or more Rr.
23. The compound of any one of embodiments 18-21, wherein R^{C} is C1-C6 heteroalkyl optionally substituted with one or more R₇.
24. The compound of any one of embodiments 18-21, wherein R^{C} is C1-C6 haloalkyl optionally substituted with one or more R₇.
25. The compound of any one of embodiments 18-21, wherein R^{C} is cycloalkyl optionally substituted with one or more R₇ (e.g., monocyclic cycloalkyl, bicyclic cycloalkyl).
26. The compound of any one of embodiments 18-21, wherein R^{C} is heterocyclyl optionally substituted with one or more R₇.
27. The compound of any one of embodiments 21-26, wherein R₇ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano.
28. The compound of any one of the preceding embodiments, wherein the compound is a compound of Formula (I-a):

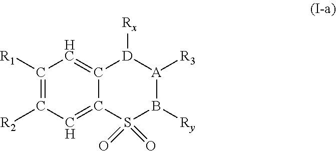

wherein:
   A is carbon (C);
   B and D are Nitrogen (N); and
   wherein either A and B or A and D are connected via a double bond; and
   wherein R₁ and R₂ are independently selected from the group consisting of hydrogen, fluorine, chlorine, bromine and iodine; and
   wherein A and B form a double bond and Rₓ is H or wherein A and D form a double bond and R_{y} is H; and
wherein R₃ is independently selected from the group consisting of:

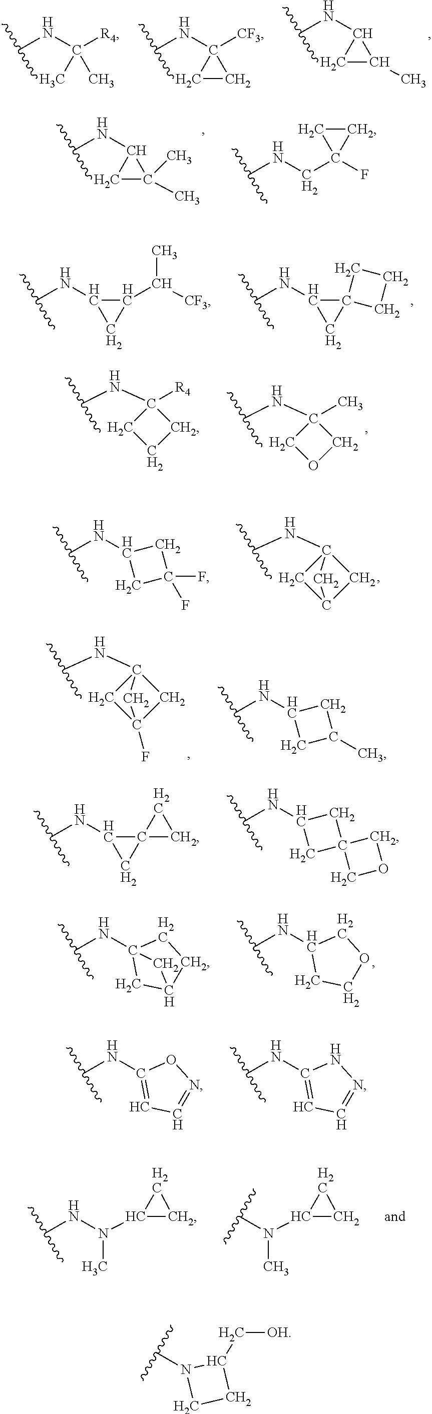

and wherein R₄ is selected from the group consisting of C1-C3 alkyl optionally substituted with one to two substituents selected from the group consisting of fluoro and hydroxy, ethynyl, C1-C2 alkoxy, (methoxy)-C1-C2 alkyl, cyano, fluoro, (methylsulfonyl)-C1-C2 alkyl, (dimethylamino)-C1-C2 alkyl and n-methyl carbamoyl.
29. The compound of any one of the preceding embodiments, wherein:
   A is carbon (C);
   B and D are Nitrogen (N); and
   wherein A and B or A and D are connected via a double or single bond; and
   wherein R₁ and R₂ are independently selected from the group consisting of hydrogen, fluorine, chlorine, bromine and iodine; and
   wherein A and B form a double bond and Rₓ is H or wherein A and D form a double bond and R_{y} is H; and
   wherein R₃ is independently selected from the group consisting of:

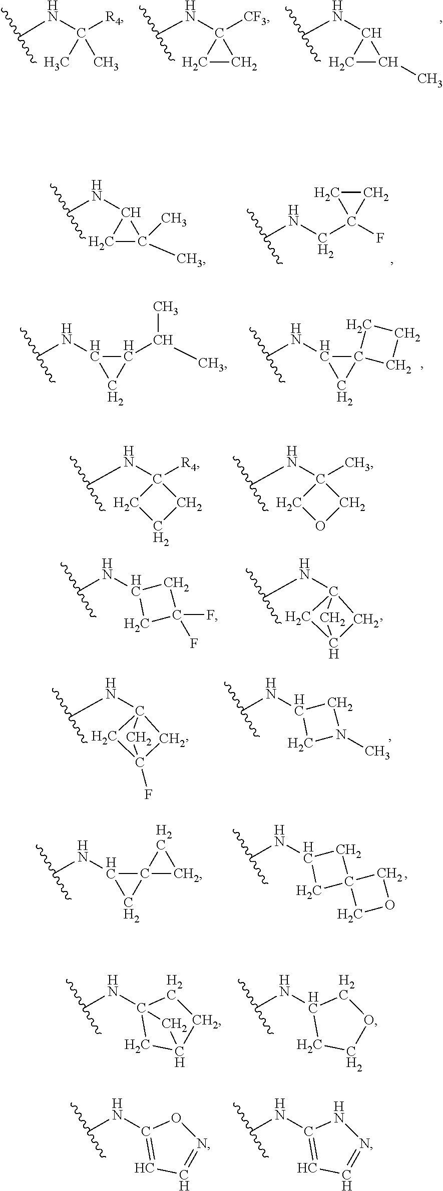

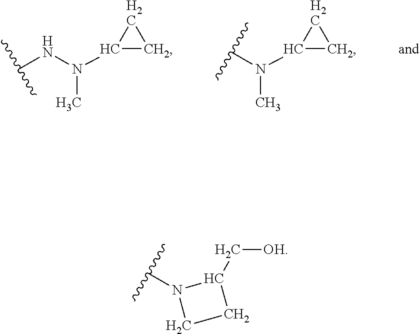

30. The compound of any one of the preceding embodiments, wherein the compound is a compound of Formula (I-b):

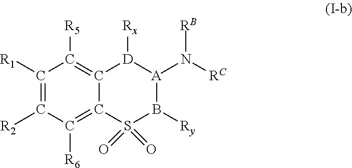

or a pharmaceutically acceptable salt, tautomer, stereoisomer, hydrate, or isotope thereof, wherein A, B, D, R₁, R₂, R₅, R₆, Rₓ R_{y}, R^{A}—R^{B}, R^{C}, and R⁷ are defined as provided for Formula (I).
31. The compound of any one of the preceding embodiments, wherein the compound is a compound of Formula (I-c):

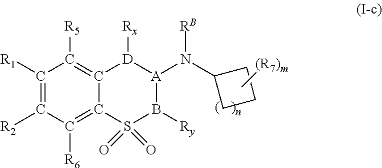

or a pharmaceutically acceptable salt, tautomer, stereoisomer, hydrate, or isotope thereof, wherein A, B, D, R₁, R₂, R₅, R₆, Rₓ R_{y}, R^{A}, R^{B}, R^{C}, and R⁷ are defined as provided for Formula (I), m is an integer between 0 and 12; and n is an integer between 0 and 4.
32. The compound of any one of the preceding embodiments, wherein the compound is a compound of Formula (I-e):

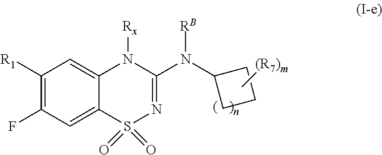

or a pharmaceutically acceptable salt, tautomer, stereoisomer, hydrate, or isotope thereof, wherein R₁, R₅, Rₓ, and R⁷ are defined as provided for Formula (I), m is an integer between 0 and 12; and n is an integer between 0 and 4.
33. The compound of any one of the preceding embodiments, wherein the compound is a compound of Formula (I-f):

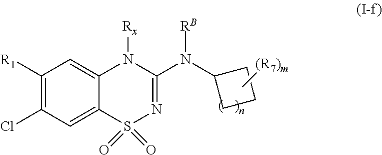

or a pharmaceutically acceptable salt, tautomer, stereoisomer, hydrate, or isotope thereof, wherein R₁, R₅, Rₓ, and R⁷ are defined as provided for Formula (I), m is an integer between 0 and 12; and n is an integer between 0 and 4.
34. The compound of any one of the preceding embodiments, wherein the compound is selected from the group consisting of

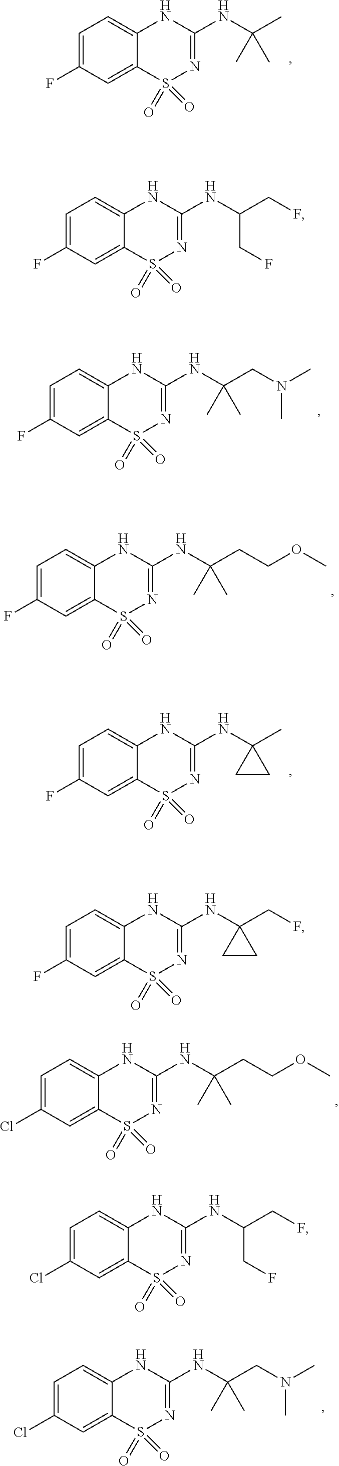

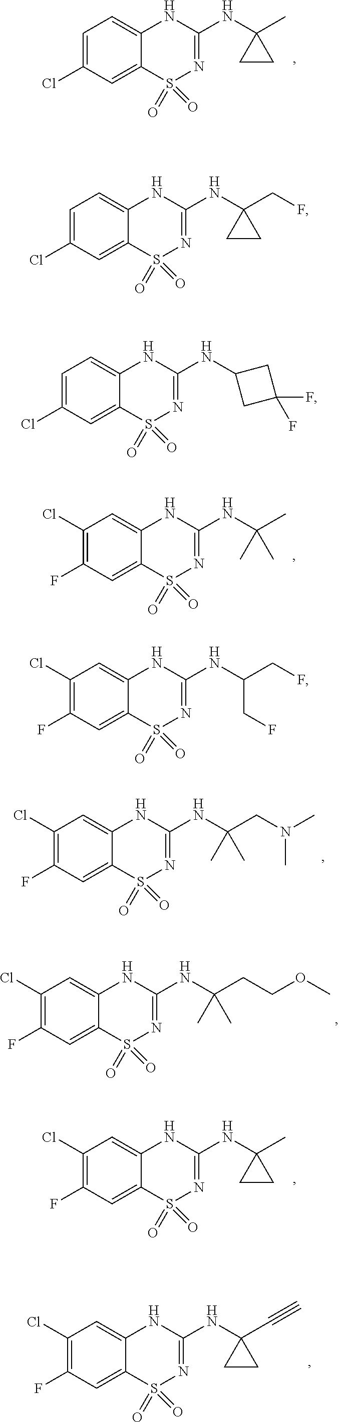

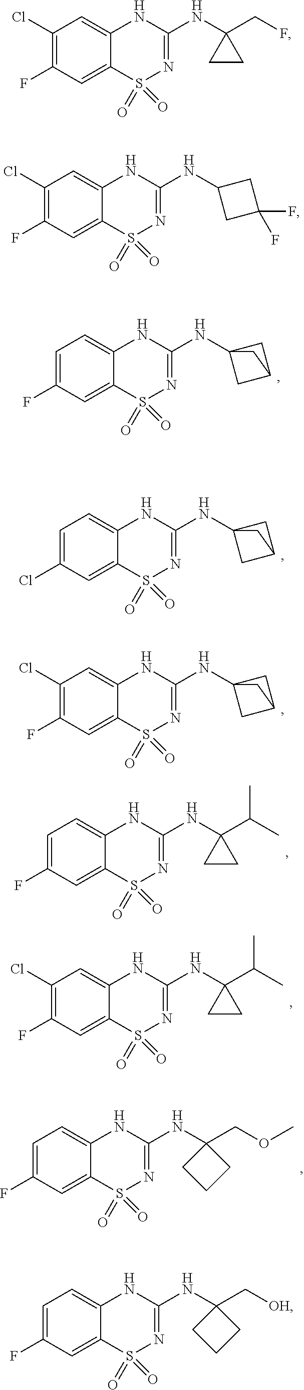

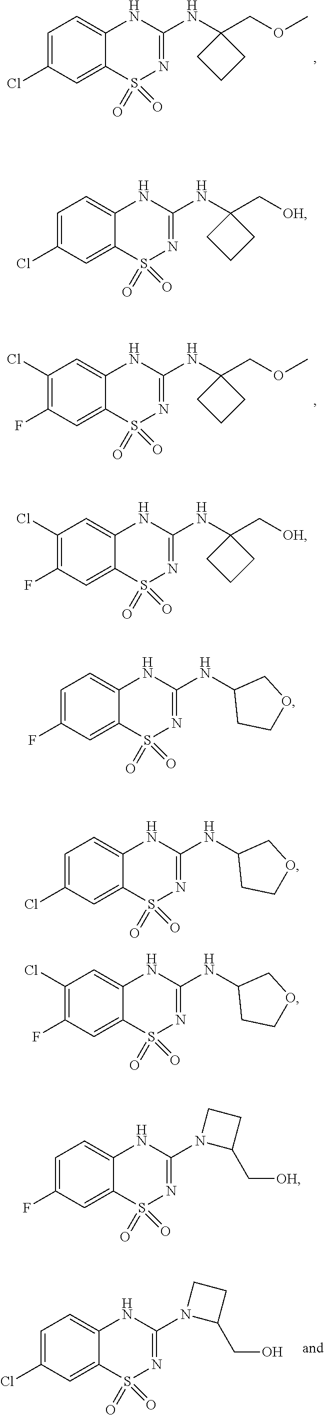

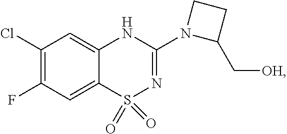 [SMILES: O=S1(=O)N=C(N2CCC2CO)Nc2cc(Cl)c(F)cc21]

or a pharmaceutically acceptable salt thereof.
35. The compound of any one of the preceding embodiments, wherein the compound is provided in Table 1 or a pharmaceutically acceptable salt thereof.
36. A pharmaceutical composition comprising a compound of any one of the preceding embodiments and a pharmaceutically acceptable excipient.
37. A method for treating a disease or disorder in a subject with a compound of any one of embodiments 1-35, or a pharmaceutically acceptable thereof, or a pharmaceutical composition of embodiment 36.
38. The method of embodiment 37, wherein the disease or disorder is selected from a metabolic disorder (e.g., CHI), cancer, a neurological disorder, a cardiovascular disorder, a pulmonary disorder, an integumentary disorder, a sexual disorder, a urinary disorder, or a symptom thereof.
39. The method of any one of embodiments 37-38, wherein the disease or disorder is a metabolic disorder.
40. The method of any one of embodiments 37-38, wherein the metabolic disorder is hyperinsulinism, e.g., congenital hyperinsulinism (CHI).
41. The method of any one of embodiments 37-40, wherein the subject has been diagnosed or identified with having the disease or disorder.
42. The method of any one of embodiments 37-42, wherein the subject is a mammal (e.g., a human).
43. The method of any one of embodiments 37-42, wherein the compound or pharmaceutical composition is formulated for oral or intravenous administration.
44. The method of embodiment 43, wherein the compound or pharmaceutical composition is a solid dosage form, in particular an oral dosage form, such as a tablet or capsule.
45. A process for preparing compounds according to any of the embodiments 1-6, wherein said process comprises the steps of:
a) providing a first compound according to formula (X)

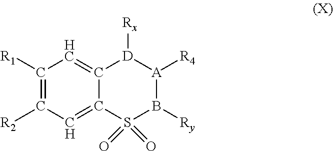

wherein:
   A is carbon (C);
   B and D are Nitrogen (N); and
   wherein A and B or A and D are connected via a double or single bond; and
   wherein R₁ and R₂ are independently selected from the group consisting of hydrogen, fluorine, chlorine, bromine and iodine; and
   wherein A and B form a double bond, Rₓ is H or wherein A and D form a double bond, R_{y} is H and
   wherein R₄ is independently selected from the group consisting of fluorine, chlorine, bromine and iodine, preferably chlorine:
b) providing a second compound according to formula H—R₃, wherein R₃ is selected from the group consisting of:

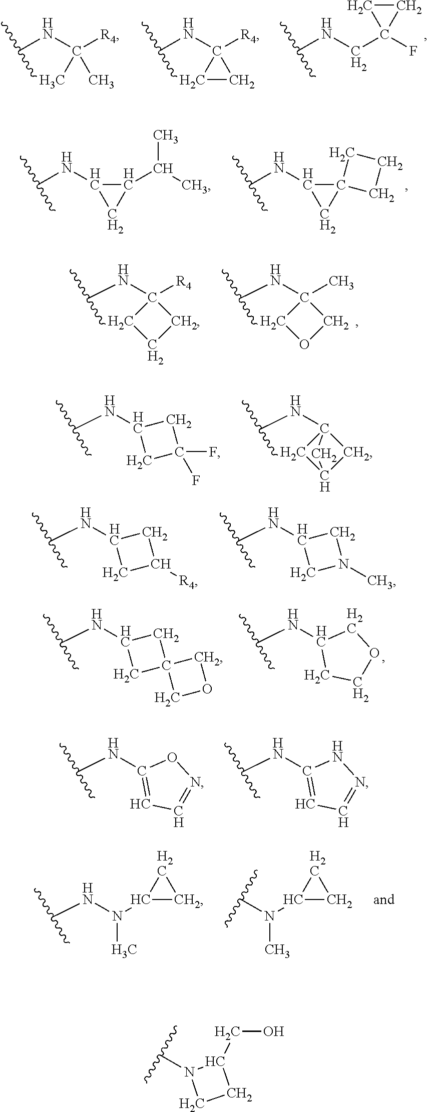

and wherein R₄ is selected from the group consisting of C1-C3 alkyl optionally substituted with one to two substituents selected from the group consisting of fluoro and hydroxy, ethynyl, C1-C2 alkoxy, (methoxy)-C1-C2 alkyl, cyano, fluoro, (methylsulfonyl)-C1-C2 alkyl, (dimethylamino)-C1-C2 alkyl and n-methyl carbamoyl;
c) reacting said first compound with said second compound under conditions that result in the formation of a compound of any one of the embodiments 1-35.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

¹H & ¹H{¹⁹F}NMR: ¹HNMR and ¹H{¹⁹F}NMR spectra were recorded on a Bruker Ultrashield (400 MHz). Chemical shifts were reported in ppm. The multiplicity of a signal is designated by the following abbreviations: br s, broad singlet; s, singlet; d, doublet; t, triplet; q, quartet; dd, doublet of doublets; dt, doublet of triplets; m, multiplet. All observed coupling constants, J, are reported in Hertz (Hz). Exchangeable protons are not always observed.

### LC/MS:

Condition 1: LC-MS data was generated using a Waters 2695 e system: Waters PDA 2998 detector, Waters QDA detector (ESI), Sedere SEDEX 80 (light scattering detector).

LC-MS method: reverse phase HPLC analysis; Column Agilent: Poroshell, Length: 100 mm, Internal diameter: 4.6 mm, Particle size: 4 μm; Solvent A: Water with Formic Acid (0.1% V/V); Solvent B: Acetonitrile; UV detection: 220 nm

### Gradient 1:

| Time (min) | Flow (mL/min) | % A | % B | Curve |
|---|---|---|---|---|
| 0 | 1 | 98 | 2 | initial |
| 2 | 1 | 98 | 2 | 6 |
| 12 | 1 | 0 | 100 | 6 |
| 15.4 | 1 | 0 | 100 | 6 |
| 16.7 | 1 | 98 | 2 | 6 |
| 18.4 | 1 | 98 | 2 | 6 |

### Condition 2:

LC/MS data was generated using a Waters Acquity UPLC Class I: Waters PDA ek detector, Waters SQD2 MS detector, Sedere SEDEX 80 (light scattering detector). LC/MS method: reverse phase HPLC analysis; Column Waters: Acquity Premier CSH C18, length: 100 mm, Internal diameter: 2.1 mm, particle size: 1.7 μm; Solvent A: Water with Formic Acid (0.1% V/V); Solvent B: Acetonitrile; UV detection: 220 nm

### Gradient 1:

| Time (min) | Flow (mL/min) | % A | % B | Curve |
|---|---|---|---|---|
| 0 | 0.4 | 98 | 2 | initial |
| 1.5 | 0.4 | 98 | 2 | Linear |
| 8.5 | 0.4 | 0 | 100 | Linear |
| 9.4 | 0.4 | 0 | 100 | Linear |
| 9.5 | 0.4 | 98 | 2 | Linear |
| 12 | 0.4 | 98 | 2 | Linear |

### Silica Gel Flash Chromatography

Condition 1, Mobile Phase: cyclohexane/EtOAc, Gradient 1: 100:0 to 0:100; Gradient 2: 100:0 to 0:100, then EtOAc 100%; Gradient 3: 100:0 to 50:50.
Condition 2, Mobile Phase: EtOAc/MeOH; Gradient 1: 100:0 to 90:10.

### Preparative HPLC

Condition 1: Column: XBridge C18 (30×150 (5 μm)); flow rate: 43 mL/min; Eluant: Water (+0.1% formic acid)/Acetonitrile; Gradient 1: 25 to 40% of acetonitrile in water (+0.1% formic acid).
Condition 2: Column: XBridge C18 (30×150 (5 μm)); flow rate: 43 mL/min; Eluant: Water (+0.1% formic acid)/MeOH; Gradient 1: from 40 to 55% of MeOH in water (+0.1% formic acid).

### Chiral SFC Separation

Condition 1: Column: Chiralpak OD-H (20×250 mm); Mobile Phase: CO₂/EtOH; Gradient 1: 75:25 isocratic; Gradient 2: 60:40 isocratic.
Condition 2: Column: Chiralpak OD-H (20×250 mm); Mobile Phase: CO₂/(EtOH+0.3% v/v i-PrNH2); Gradient 1: 70:30 isocratic.
Condition 3: Column: Chiralpak OD-H (20×250 mm); Mobile Phase: CO₂/MeOH; Gradient 1: 75:25 isocratic.

### Chiral SFC Purity Analysis Conditions

### Chiral SFC Purity Analysis Conditions for Enantiomers Separation

Column: ChiralPak AS-3 (4.6×100 mm); Column Temperature: 35° C.; Flow Rate: 3.5 mL/min;

Detector Wavelength: 220-410 nm; Injection Volume: 2 μL; BPR: 1500 PSI;
Condition 1: Mobile Phase: CO₂/MeOH; Gradient 1: 80:20 isocratic; Gradient 2: 75/25 isocratic.
Condition 2: Mobile Phase: CO2/(EtOH+0.3% c/v i-PrNH2); Gradient 1: 70/30 isocratic
Condition 3: Mobile Phase: CO₂/EtOH; Gradient 1: 70:30 isocratic;

### Chiral SFC Purity Analysis Conditions for Regioisomers Separation

Column Details: ChiralPak OD-3 (4.6×100 mm); Column Temperature: 35° C.; Flow Rate: 3.5 mL/min; Detector Wavelength: 220-410 nm; Injection Volume: 2 μL; BPR: 1500 PSI;
Condition 1: Mobile Phase CO₂/EtOH; Gradient 1: 75:25 isocratic.

### Example 1. Synthesis of 3,7-dichloro-4H-benzo[e][1,2,4]thiadiazine 1,1-dioxide

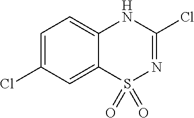 [SMILES: O=S1(=O)N=C(Cl)Nc2ccc(Cl)cc21]

The title compound was obtained according to the method previously described by H. Nishimura et al. (Patent, Dainippon Pharmaceutical Co, U.S. Pat. No. 4,029,780, 1977) starting from the corresponding 7-chloro-2H-benzo[e][1,2,4]thiadiazin-3 (4H)-one 1,1-dioxide and chlorination with phosphorus oxychloride.

### Example 2. Synthesis of 3-chloro-7-fluoro-4H-benzo[e][1,2,4]thiadiazine 1,1-dioxide

### 2.1. Preparation of 7-fluoro-2H-benzo[e][1,2,4]thiadiazin-3 (4H)-one 1,1-dioxide

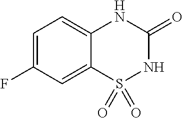 [SMILES: O=C1Nc2ccc(F)cc2S(=O)(=O)N1]

2-amino-5-fluorobenzene-1-sulfonamide (2.5 g, 13.1 mmol) and urea (0.9 g, 14.5 mmol) were intimately mixed and heated to 200° C. for 2 hours. Ammonia was evolved, and the mixture suddenly crystallized. The crude solid was dissolved in a solution of 1 N NaOH (30 mL) and stirred for 5 min. The pH was adjusted to 1-2 with a solution of 1 N HCl (40 mL). The precipitate was collected by filtration, washed with water, and dried under vacuum to afford the title compound (2.6 g, 83%) as a white solid. LC-MS (ESI+): m/z 217.1 [M+H]⁺. ¹H-NMR (400 MHz, DMSO-d6): δ 11.35 (s, 1H), 7.67 (dd, J=2.8 Hz, J=7.6 Hz, 1H), 7.57-7.51 (m, 1H), 7.28 (dd, J=4.4 Hz, J=9.0 Hz, 1H), and one NH missing.

### 2.2. Preparation of 3-chloro-7-fluoro-4H-benzo[e][1,2,4]thiadiazine 1,1-dioxide

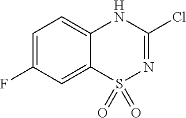 [SMILES: O=S1(=O)N=C(Cl)Nc2ccc(F)cc21]

A suspension of 7-fluoro-2H-benzo[e][1,2,4]thiadiazin-3 (4H)-one 1,1-dioxide (2.4 g, 10.1 mmol) in phosphorus oxychloride (14.1 mL, 151.5 mmol) was stirred at 120° C. for 20 hours. The mixture was cooled to RT and poured into ice water (50 mL), and stirred for 30 min. A solution of 3 N NaOH (350-400 mL) was added dropwise at 0° C. until pH 9 over 30 min. The mixture was extracted with EtOAc (3×200 mL). The organic layers were combined, dried over anhydrous Na₂SO₄, filtered, and concentrated in vacuo to afford the title compound as a white powder (2.1 g, 88%). The product was used without further purification. LC-MS (ESI+): m/z 235.1 [M+H]⁺. ¹H-NMR (400 MHz, DMSO-d6): δ 7.38 (dd, J=2.9 Hz, J=7.8 Hz, 1H), 7.36-7.30 (m, 1H), 7.19 (dd, J=5.0 Hz, J=9.0 Hz, 1H), and one NH missing.

### Example 3. Synthesis of 3,6-dichloro-7-fluoro-4H-benzo[e][1,2,4]thiadiazine 1,1-dioxide

### 3.1. Preparation of 2-amino-4-chloro-5-fluorobenzenesulfonamide

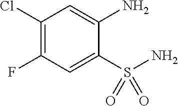 [SMILES: Nc1cc(Cl)c(F)cc1S(N)(=O)=O]

The title compound was obtained according to the method previously described by P. De Tullio et al. (J. Med Chem. 2005, 48, 4990-5000) starting from the corresponding 3-chloro-4-fluoroaniline. 3.2. Preparation of 6-chloro-7-fluoro-2H-benzo[e][1,2,4]thiadiazin-3 (4H)-one 1,1-dioxide

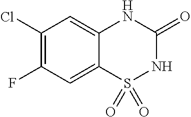 [SMILES: O=C1Nc2cc(Cl)c(F)cc2S(=O)(=O)N1]

2-amino-4-chloro-5-fluorobenzenesulfonamide (3.0 g, 13.4 mmol) and urea (0.9 g, 14.5 mmol) were intimately mixed and heated to 200° C. for 1 hour. Ammonia was evolved, and the mixture suddenly crystallized. The crude solid was dissolved in a solution of 1 N NaOH (30 mL) and stirred for 5 min. The pH was adjusted to 1-2 with a solution of 1 N HCl (40 mL). The precipitate was collected by filtration, washed with water, and dried under vacuum to afford the title compound (1.8 g, 53%) as a purple solid. LC-MS (ESI+): m/z 251.1 [M+H]⁺. ¹H-NMR (400 MHz, DMSO-d6): δ 10.27 (s, 1H), 7.62 (d, J=7.9 Hz, 1H), 7.23 (d, J=6.1 Hz, 1H), and one NH missing.

### 3.3. Preparation of 3,6-dichloro-7-fluoro-4H-benzo[e][1,2,4]thiadiazine 1,1-dioxide

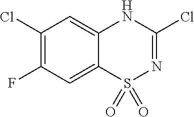 [SMILES: O=S1(=O)N=C(Cl)Nc2cc(Cl)c(F)cc21]

To a solution of 6-chloro-7-fluoro-2H-benzo[e][1,2,4]thiadiazin-3 (4H)-one 1,1-dioxide (3.6 g, 14.4 mmol) in phosphorus oxychloride (20.0 mL, 215.4 mmol) was added N,N-diethylaniline (4.3 g, 28.7 mmol, 4.6 mL). The solution was heated to 120° C. for 18 hours, then cooled to 25° C., poured into ice water (50 mL), and stirred for 1 hour. The precipitate was collected by filtration, washed with water, and dried under vacuum. The residue was purified by silica-gel column chromatography (cyclohexane/EtOAc=100/0:0/100) to give 2.63 g (68%) of 3,6-dichloro-7-fluoro-4H-benzo[e][1,2,4]thiadiazine 1,1-dioxide as a pale brown solid. LC-MS (ESI+): m/z 269.0 [M+H]⁺. ¹H-NMR (400 MHz, DMSO-d6): δ 8.05 (d, J=8.1 Hz, 1H), 7.50 (d, J=6.2 Hz, 1H), and one NH missing.

### Example 4. General Procedures for the Preparation of 3-(alkylamino)-4H-benzo[e][1,2,4]thiadiazine 1,1-dioxides

### 4.1. Method A:

A mixture of the appropriate 3-chloro-substituted benzothiadiazine 1,1-dioxide (0.5 mmol, 1 eq.) and the appropriate alkylamine (50 eq.) was heated in a sealed vessel at 150° C. for 50-60 hours (until completion of the reaction monitored by LC-MS). The excess of amine was removed by distillation under reduced pressure. The crude residue was purified by silica gel column chromatography (cyclohexane/EtOAc=100/0:0/100 then EtOAc=100%) to give the title compound (yields: 30-40%) as a white powder.

### 4.2. Method B:

A solution of the appropriate 3-chloro-substituted benzothiadiazine 1,1-dioxide (0.5 mmol, 1 eq.), the appropriate alkylamine hydrochloride (2 eq.) and N,N-Diisopropylethylamine (3 eq.), in 2 mL of dioxane, was heated in a sealed vessel at 120° C. for 2-20 hours (until completion of the reaction monitored by LC-MS). The mixture was diluted with water (25 mL), extracted with EtOAc (25 mL×3), and the combined organic extracts were washed with brine, dried over anhydrous Na₂SO₄, filtered and concentrated. The residue was purified by silica gel column chromatography (cyclohexane/EtOAc=100/0:0/100 then EtOAc=100%) to give the title compound (yields: 20-66%) as a white powder.

### 4.3. Method C:

A solution of the appropriate 3-chloro-substituted benzothiadiazine 1,1-dioxide (0.5 mmol, 1 eq.), the appropriate alkylamine hydrochloride (2 eq.) and N,N-Diisopropylethylamine (3 eq.), in 2 mL of dioxane, was heated in a sealed vessel at 60° C. for 2-3 hours (until completion of the reaction monitored by LC-MS). The mixture was diluted with water (25 mL), extracted with EtOAc (25 mL×3), and the combined organic extracts were washed with brine, dried over anhydrous Na₂SO₄, filtered and concentrated. The residue was purified by silica gel column chromatography (cyclohexane/EtOAc=100/0:0/100 then EtOAc=100%) to give the title compound (yields: 33-65%) as a white powder.

### 4.4. Method D:

A solution of the appropriate 3-chloro-substituted benzothiadiazine 1,1-dioxide (0.5 mmol, 1 eq.), the appropriate alkylamine (2 eq.) and N,N-Diisopropylethylamine (1 eq.), in 2 mL of dioxane, was heated in a sealed vessel at 100° C. for 2-5 hours (until completion of the reaction monitored by LC-MS). The mixture was diluted with water (25 mL), extracted with EtOAc (25 mL×3), and the combined organic extracts were washed with brine, dried over anhydrous Na₂SO₄, filtered and concentrated. The residue was purified by silica gel column chromatography (cyclohexane/EtOAc=100/0:0/100 then EtOAc=100%) to give the title compound (yields: 8-45%) as a white powder.

### 4.5. Method E:

A solution of the appropriate 3-chloro-substituted benzothiadiazine 1,1-dioxide (0.5 mmol, 1 eq.), the appropriate alkylamine (2 eq.) and N,N-Diisopropylethylamine (1 eq.), in 2 mL of dioxane, was heated in a sealed vessel at 60° C. for 2-3 hours (until completion of the reaction monitored by LC-MS). The mixture was diluted with water (25 mL), extracted with EtOAc (25 mL×3), and the combined organic extracts were washed with brine, dried over anhydrous Na₂SO₄, filtered and concentrated. The residue was purified by silica gel column chromatography (cyclohexane/EtOAc=100/0:0/100 then EtOAc=100%) to give the title compound (yields: 45-70%) as a white powder.

### 4.6. Method F:

A solution of the appropriate 3-chloro-substituted benzothiadiazine 1,1-dioxide (0.5 mmol, 1 eq.) and the appropriate alkylamine (20 eq.) in 2 mL of dioxane, was heated in a sealed vessel at 120° C. for 18-24 hours (until completion of the reaction monitored by LC-MS). The mixture was diluted with water (25 mL), extracted with EtOAc (25 mL×3), and the combined organic extracts were washed with brine, dried over anhydrous Na₂SO₄, filtered and concentrated. The residue was purified by silica gel column chromatography (cyclohexane/EtOAc=100/0:0/100 then EtOAc=100%) to give the title compound (yields: 40-50%) as a white powder.

### 4.7. Method G:

A solution of the appropriate 3-choro-substituted benzothiadiazine 1,1-dioxide (0.5 mmol, 1 eq.) and the appropriate alkylamine (20 eq.) in 2 mL of dioxane, was heated in a sealed vessel at 40° C. for 4-5 days (until completion of the reaction monitored by LC-MS). The mixture was diluted with water (25 mL), extracted with EtOAc (25 mL×3), and the combined organic extracts were washed with brine, dried over anhydrous Na₂SO₄, filtered and concentrated. The residue was purified by silica gel column chromatography (cyclohexane/EtOAc=100/0:0/100 then EtOAc=100%) to give the title compound (yields: 21627.) as a white powder.

The list of certain compounds and their data of the present invention is set forth in Table 2, below.

TABLE 2
Exemplary compounds and characterization

| | | | LC-MS | |
|---|---|---|---|---|
| | | | (ESI+): | |
| Method | Structure | Name | [M + H]⁺ | ¹H-NMR |
| A, F | 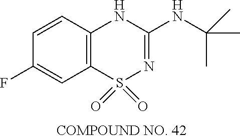 [SMILES: CC(C)(C)NC1=NS(=O)(=O)c2cc(F)ccc2N1] | 3-(tert-butylamino)- 7-fluoro-4H- benzo[e][1,2,4] thiadiazine 1,1-dioxide | 272.1 | ¹H-NMR (400 MHz, DMSO-d6): δ 10.24 (s, 1H), 7.50-7.41 (m, 2H), 7.15 (dd, J = 4.4 Hz, J = 9.0 Hz, 1H), 6.76 (s, 1H), 1.38 (s, 9H). |
| A, F | 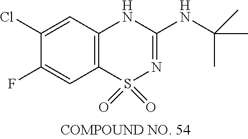 [SMILES: CC(C)(C)NC1=NS(=O)(=O)c2cc(F)c(Cl)cc2N1] | 3-(tert-butylamino)- 6-chloro-7- fluoro-4H- benzo[e][1,2,4] thiadiazine 1,1-dioxide | 306.1 | ¹H-NMR (400 MHz, DMSO-d6): δ 10.23 (s, 1H), 7.75 (d, J = 7.8 Hz, 1H), 7.37 (d, J = 6.0 Hz, 1H), 6.98 (s, 1H), 1.38 (s, 9H). |
| B | 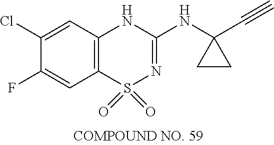 [SMILES: C#CC1(NC2=NS(=O)(=O)c3cc(F)c(Cl)cc3N2)CC1] | 6-chloro-3-((1- ethynylcyclopropyl) amino)- 7-fluoro-4H- benzo[e][1,2,4] thiadiazine 1,1-dioxide | 314.2 | ¹H-NMR (400 MHz, DMSO-d6): δ 10.66 (s, 1H), 8.34 (br s, 1H), 7.79 (d, J = 8.3 Hz, 1H), 7.63 (br s, 1H), 3.17 (s, 1H), 1.28-1.12 (m, 4H). |
| E | 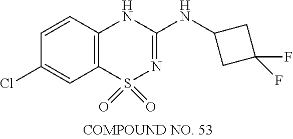 [SMILES: O=S1(=O)N=C(NC2CC(F)(F)C2)Nc2ccc(Cl)cc21] | 7-chloro-3-((3,3- difluorocyclobutyl) amino)-4H- benzo[e][1,2,4] thiadiazine 1,1-dioxide | 322.2 | ¹H-NMR (400 MHz, DMSO-d6): δ 10.84 (s, 1H), 7.84 (s, 1H), 7.68 (d, J = 2.3 Hz, 1H), 7.63 (dd, J = 2.3 Hz, J = 8.8 Hz, 1H), 7.29 (d, J = 8.8 Hz, 1H), 4.19-4.09 (m, 1H), 3.04-2.93 (m, 2H), 2.75-2.62 (m, 2H). |
| E | 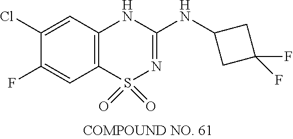 [SMILES: O=S1(=O)N=C(NC2CC(F)(F)C2)Nc2cc(Cl)c(F)cc21] | 6-chloro-3-((3,3- difluorocyclobutyl) amino)-7-fluoro- 4H- benzo[e][1,2,4] hiadiazine 1,1-dioxide | 340.1 | ¹H-NMR (400 MHz, DMSO-d6): δ 10.75 (s, 1H), 7.97 (s, 1H), 7.78 (d, J = 7.9 Hz, 1H), 7.49 (d, J = 5.6 Hz, 1H), 4.17-4.09 (m, 1H), 3.04-2.94 (m, 2H), 2.75- 2.62 (m, 2H). |
| B | 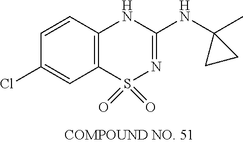 [SMILES: CC1(NC2=NS(=O)(=O)c3cc(Cl)ccc3N2)CC1] | 7-chloro-3-((1- methylcyclopropyl) amino)-4H- benzo[e][1,2,4] thiadiazine 1,1-dioxide | 287.0 | ¹H-NMR (400 MHz, DMSO-d6): δ 10.44 (s, 1H), 7.71-7.59 (m, 3H), 7.32 (s, 1H), 1.37 (s, 3H), 0.77-0.68 (m, 4H). |
| B | 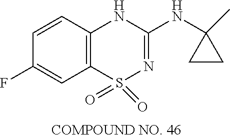 [SMILES: CC1(NC2=NS(=O)(=O)c3cc(F)ccc3N2)CC1] | 7-fluoro-3-((1- methylcyclopropyl) amino)-4H- benzo[e][1,2,4] thiadiazine 1,1-dioxide | 270.5 | ¹H-NMR (400 MHz, DMSO-d6): δ 10.38 (s, 1H), 7.63 (s, 1H), 7.50-7.42 (m, 2H), 7.35 (s, 1H), 1.37 (s, 3H), 0.77-0.68 (m, 4H). |
| B | 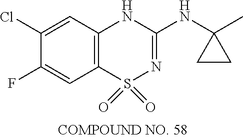 [SMILES: CC1(NC2=NS(=O)(=O)c3cc(F)c(Cl)cc3N2)CC1] | 6-chloro-7- fluoro-3-((1- methylcyclopropyl) amino)-4H- benzo[e][1,2,4] thiadiazine 1,1-dioxide | 304.2 | ¹H-NMR (400 MHz, DMSO-d6): δ 10.35 (s, 1H), 7.89 (br s, 1H), 7.75 (d, J = 7.7 Hz, 1H), 7.58 (br s, 1H), 1.37 (s, 3H), 0.78-0.69 (m, 4H). |
| B | 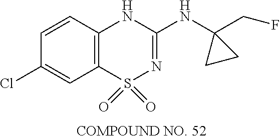 [SMILES: O=S1(=O)N=C(NC2(CF)CC2)Nc2ccc(Cl)cc21] | 7-chloro-3-((1- (fluoromethyl) cyclopropyl) amino)-4H- benzo[e][1,2,4] thiadiazine 1,1-dioxide | 304.2 | ¹H-NMR (400 MHz, DMSO-d6): δ 10.56 (s, 1H), 7.89 (s, 1H), 7.68 (d, J = 2.3 Hz, 1H), 7.62 (dd, J = 2.3 Hz, J = 8.7 Hz, 1H), 7.32 (d, J = 8.7 Hz, 1H), 4.47 (d, J = 48.9 Hz, 2H), 1.01-0.89 (m, 4H). |
| B | 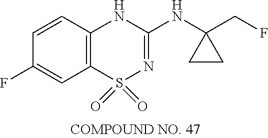 [SMILES: O=S1(=O)N=C(NC2(CF)CC2)Nc2ccc(F)cc21] | 7-fluoro-3-((1- (fluoromethyl) cyclopropyl) amino)-4H- benzo[e][1,2,4] thiadiazine 1,1-dioxide | 288.3 | ¹H-NMR (400 MHz, DMSO-d6): δ 10.49 (s, 1H), 7.84 (s, 1H), 7.53-7.44 (m, 2H), 7.35 (m, 1H), 4.47 (d, J = 49.0 Hz, 2H), 1.01-0.90 (m, 4H). |
| B | 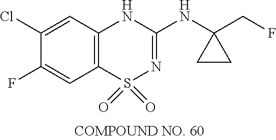 [SMILES: O=S1(=O)N=C(NC2(CF)CC2)Nc2cc(Cl)c(F)cc21] | 6-chloro-7-fluoro- 3-((1-(fluoromethyl) cyclopropyl) amino)-4H- benzo[e][1,2,4] thiadiazine 1,1-dioxide | 322.2 | ¹H-NMR (400 MHz, DMSO-d6): δ 10.47 (s, 1H), 8.10 (s, 1H), 7.78 (d, J = 7.9 Hz, 1H), 7.58 (s, 1H), 4.46 (d, J = 48.9 Hz, 2H), 1.01- 0.90 (m, 4H). |
| B | 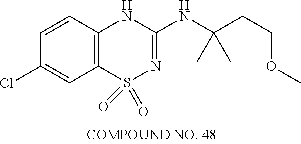 [SMILES: COCCC(C)(C)NC1=NS(=O)(=O)c2cc(Cl)ccc2N1] | 7-chloro-3- ((4-methoxy- 2-methylbutan- 2-yl)amino)-4H- benzo[e][1,2,4] thiadiazine 1,1-dioxide | 332.2 | ¹H-NMR (400 MHz, DMSO-d6): δ 10.41 (s, 1H), 7.66 (d, J = 2.4 Hz, 1H), 7.60 (dd, J = 2.4 Hz, J = 8.7 Hz, 1H), 7.14 (d, J = 8.7 Hz, 1H), 6.79 (s, 1H), 3.41 (t, J = 6.8 Hz, 2H), 3.23 (s, 3H), 2.00 (t, J = 6.8 Hz, 2H), 1.38 (s, 6H). |
| B | 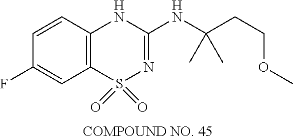 [SMILES: COCCC(C)(C)NC1=NS(=O)(=O)c2cc(F)ccc2N1] | 7-fluoro-3- ((4-methoxy- 2-methylbutan- 2-yl)amino)-4H- benzo[e][1,2,4] thiadiazine 1,1-dioxide | 316.7 | ¹H-NMR (400 MHz, DMSO-d6): δ 10.37 (s, 1H), 7.49 (dd, J = 2.9 Hz, J = 7.5 Hz, 1H), 7.44 (td, J = 2.9 Hz, J = 8.8 Hz, 1H), 7.16 (dd, J = 4.3 Hz, J = 8.8 Hz, 1H), 6.76 (s, 1H), 3.41 (t, J = 6.8 Hz, 2H), 3.23 (s, 3H), 2.00 (t, J = 6.8 Hz, 2H), 1.37 (s, 6H). |
| B | 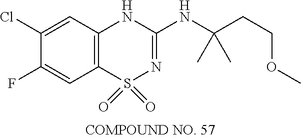 [SMILES: COCCC(C)(C)NC1=NS(=O)(=O)c2cc(F)c(Cl)cc2N1] | 6-chloro-7-fluoro- 3-((4-methoxy-2- methylbutan- 2-yl)amino)-4H- benzo[e][1,2,4] thiadiazine 1,1-dioxide | 350.2 | ¹H-NMR (400 MHz, DMSO-d6): δ 10.35 (s, 1H), 7.76 (d, J = 8.0 Hz, 1H), 7.36 (d, J = 6.0 Hz, 1H), 6.95 (s, 1H), 3.41 (t, J = 6.8 Hz, 2H), 3.22 (s, 3H), 1.99 (t, J = 6.8 Hz, 2H), 1.37 (s, 6H). |
| C | 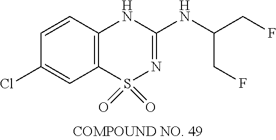 [SMILES: O=S1(=O)N=C(NC(CF)CF)Nc2ccc(Cl)cc21] | 7-chloro-3-((1,3- difluoropropan-2- yl)amino)-4H- benzo[e][1,2,4] thiadiazine 1,1-dioxide | 310.2 | ¹H-NMR (400 MHz, DMSO-d6): δ 10.73 (s, 1H), 7.70 (d, J = 2.4 Hz, 1H), 7.64 (dd, J = 2.4 Hz, J = 8.8 Hz, 1H), 7.58 (d, J = 6.0 Hz, 1H), 7.26 (d, J = 8.8 Hz, 1H), 4.69-4.62 (m, 2H), 4.57-4.50 (m, 2H), 4.45-4.29 (m, 1H). |
| C | 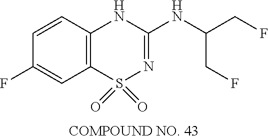 [SMILES: O=S1(=O)N=C(NC(CF)CF)Nc2ccc(F)cc21] | 3-((1,3- difluoropropan-2- yl)amino)-7- fluoro-4H- benzo[e][1,2,4] thiadiazine 1,1-dioxide | 294.3 | ¹H-NMR (400 MHz, DMSO-d6): δ 10.67 (s, 1H), 7.55-7.46 (m, 3H), 7.28 (dd, J = 4.4 Hz, J = 9.0 Hz, 1H), 4.70-4.63 (m, 2H), 4.58- 4.51 (m, 2H), 4.46-4.30 (m, 1H). |
| B | 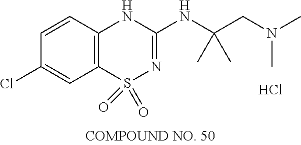 [SMILES: CN(C)CC(C)(C)NC1=NS(=O)(=O)c2cc(Cl)ccc2N1 ; Cl] | 7-chloro-3-((1- (dimethylamino)-2- methylpropan- 2-yl)amino)-4H- benzo[e][1,2,4] thiadiazine 1,1-dioxide hydrochloride salt | 331.3 | ¹H-NMR (400 MHz, DMSO-d6): δ 11.49 (br s, 1H), 9.43 (br s, 1H), 7.72 (d, J = 2.3 Hz, 1H), 7.64 (dd, J = 2.3 Hz, J = 8.8 Hz, 1H), 7.39 (br s, 1H), 7.20 (d, J = 8.7 Hz, 1H), 3.64 (d, J = 4.4 Hz, 2H), 2.84 (d, J = 4.9 Hz, 6H), 1.46 (s, 6H). |
| B | 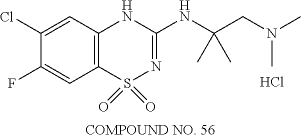 [SMILES: CN(C)CC(C)(C)NC1=NS(=O)(=O)c2cc(F)c(Cl)cc2N1 ; Cl] | 6-chloro-3-((1- (dimethylamino)- 2-methylpropan-2- yl)amino)-7-fluoro- 4H-benzo[e][1,2,4] thiadiazine 1,1-dioxide hydrochloride salt | 349.3 | ¹H-NMR (400 MHz, DMSO-d6): δ 11.56 (br s, 1H), 9.51 (br s, 1H), 7.82 (d, J = 7.9 Hz, 1H), 7.47 (br s, 1H), 7.39 (d, J = 6.0 Hz, 1H), 3.64 (d, J = 4.4 Hz, 2H), 2.84 (d, J = 4.8 Hz,6H), 1.46 (s, 6H). |
| B | 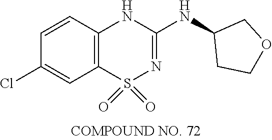 [SMILES: O=S1(=O)N=C(N[C@@H]2CCOC2)Nc2ccc(Cl)cc21] | (R)-7-chloro-3- ((tetrahydrofuran- 3-yl)amino)- 4H- benzo[e][1,2,4] thiadiazine 1,1-dioxide | 302.1 | ¹H-NMR (400 MHz, DMSO-d6): δ 10.50 (s, 1H), 7.67 (d, J = 2.3 Hz, 1H), 7.61 (dd, J = 2.3 Hz, J = 8.7 Hz, 1H), 7.52 (br s, 1H), 7.25 (d, J = 8.7 Hz, 1H), 4.37-4.30 (m, 1H), 3.86-3.78 (m, 2H), 3.74-3.68 (m, 1H), 3.59 (dd, J = 3.3 Hz, J = 9.2 Hz 1H), 2.25-2.16 (m, 1H), 1.87-1.80 (m, 1H). |
| B | 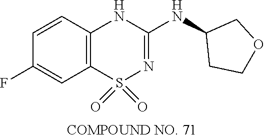 [SMILES: O=S1(=O)N=C(N[C@@H]2CCOC2)Nc2ccc(F)cc21] | (R)-7-fluoro-3- ((tetrahydrofuran- 3-yl)amino)-4H- benzo[e][1,2,4] thiadiazine 1,1-dioxide | 286.3 | ¹H-NMR (400 MHz, DMSO-d6): δ 10.43 (s, 1H), 7.52-7.43 (m, 3H), 7.27 (dd, J = 4.4 Hz, J = 8.9 Hz, 1H), 4.37-4.30 (m, 1H), 3.86-3.78 (m, 2H), 3.74-3.69 (m, 1H), 3.59 (dd, J = 3.3 Hz, J = 9.2 Hz, 1H), 2.25-2.16 (m, 1H), 1.87-1.79 (m, 1H). |
| B | 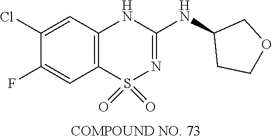 [SMILES: O=S1(=O)N=C(N[C@@H]2CCOC2)Nc2cc(Cl)c(F)cc21] | (R)-6-chloro- 7-fluoro-3- ((tetrahydrofuran- 3-yl)amino)-4H- benzo[e][1,2,4] thiadiazine 1,1-dioxide | 320.3 | ¹H-NMR (400 MHz, DMSO-d6): δ 10.42 (s, 1H), 7.77 (d, J = 7.9 Hz, 1H), 7.70 (br s, 1H), 7.48 (d, J = 6.0 Hz, 1H), 4.36-4.29 (m, 1H), 3.86-3.78 (m, 2H), 3.74-3.68 (m, 1H), 3.59 (dd, J = 3.4 Hz, J = 9.2 Hz, 1H), 2.25-2.16 (m, 1H), 1.87-1.80 (m, 1H). |
| B | 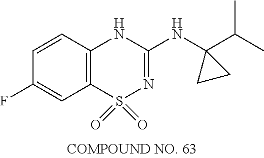 [SMILES: CC(C)C1(NC2=NS(=O)(=O)c3cc(F)ccc3N2)CC1] | 7-fluoro-3-((1- isopropylcyclopropyl) amino)-4H- benzo[e][1,2,4] thiadiazine 1,1-dioxide | 298.6 | ¹H-NMR (400 MHz, DMSO-d6): δ 10.15 (br s, 1H), 7.49-7.42 (m, 2H), 7.30 (br s, 2H), 1.77-1.67 (m, 1H), 0.90 (d, J = 6.7 Hz, 6H), 0.75 (br s, 4H). |
| B | 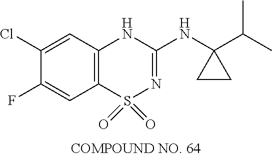 [SMILES: CC(C)C1(NC2=NS(=O)(=O)c3cc(F)c(Cl)cc3N2)CC1] | 6-chloro-7- fluoro-3-((1- isopropylcyclopropyl) amino)-4H- benzo[e][1,2,4] thiadiazine 1,1-dioxide | 332.0 | ¹H-NMR (400 MHz, DMSO-d6): δ 10.16 (br s, 1H), 7.74 (d, J = 7.9 Hz, 1H), 7.69-7.37 (m, 2H), 1.77-1.67 (m, 1H), 0.89 (d, J = 6.7 Hz, 6H), 0.79-0.73 (m, 4H). |
| B | 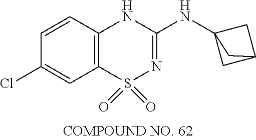 [SMILES: O=S1(=O)N=C(NC23CC(C2)C3)Nc2ccc(Cl)cc21] | 3-(bicyclo[1.1.1] pentan-1- ylamino)-7- chloro-4H- benzo[e][1,2,4] thiadiazine 1,1-dioxide | 298.2 | ¹H-NMR (400 MHz, DMSO-d6): δ 10.52 (s, 1H), 7.86 (s, 1H), 7.67 (d, J = 2.4 Hz, 1H), 7.60 (dd, J = 2.4 Hz, J = 8.8 Hz, 1H), 7.23 (d, J = 8.8 Hz, 1H), 2.47 (s, 1H), 2.09 (s, 6H). |
| B | 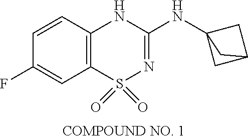 [SMILES: O=S1(=O)N=C(NC23CC(C2)C3)Nc2ccc(F)cc21] | 3-(bicyclo[1.1.1] pentan-1- ylamino)-7- fluoro-4H- benzo[e][1,2,4] thiadiazine 1,1-dioxide | 282.0 | ¹H-NMR (400 MHz, DMSO-d6): δ 10.44 (s, 1H), 7.80 (s, 1H), 7.50 (dd, J = 2.9 Hz, J = 7.6 Hz, 1H), 7.45 (dt, J = 2.9 Hz, J = 8.8 Hz, 1H), 7.25 (dd, J = 4.4 Hz, J = 8.8 Hz, 1H), 2.47 (s, 1H), 2.08 (s, 6H). |
| B | 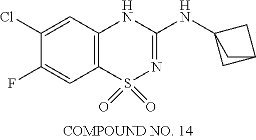 [SMILES: O=S1(=O)N=C(NC23CC(C2)C3)Nc2cc(Cl)c(F)cc21] | 3-(bicyclo[1.1.1] pentan-1- ylamino)-6- chloro-7-fluoro- 4H-benzo[e][1,2,4] thiadiazine 1,1-dioxide | 316.1 | ¹H-NMR (400 MHz, DMSO-d6): δ 10.43 (s, 1H), 8.05 (s, 1H), 7.50 (d, J = 7.9 Hz, 1H), 7.46 (d, J = 6.1 Hz, 1H), 2.48 (s, 1H), 2.09 (s, 6H). |
| B | 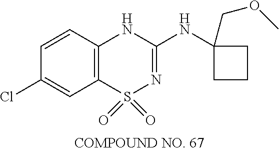 [SMILES: COCC1(NC2=NS(=O)(=O)c3cc(Cl)ccc3N2)CCC1] | 7-chloro-3-((1- (methoxymethyl) cyclobutyl) amino)-4H- benzo[e][1,2,4] thiadiazine 1,1-dioxide | 330.2 | ¹H-NMR (400 MHz, DMSO-d6): δ 10.44 (br s, 1H), 7.66 (d, J = 2.2 Hz, 1H), 7.60 (dd, J = 2.3 Hz, J = 8.7 Hz, 1H), 7.27 (br s, 1H), 7.19 (d, J = 8.7 Hz, 1H), 3.62 (s, 2H), 3.32 (s, 3H), 2.33-2.25 (m, 2H), 2.14-2.07 (m, 2H), 1.92-1.73 (m, 2H). |
| B | 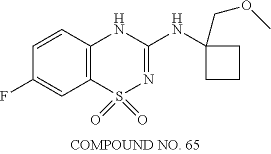 [SMILES: COCC1(NC2=NS(=O)(=O)c3cc(F)ccc3N2)CCC1] | 7-fluoro-3-((1- (methoxymethyl) cyclobutyl) amino)-4H- benzo[e][1,2,4] thiadiazine 1,1-dioxide | 314.4 | ¹H-NMR (400 MHz, DMSO-d6): δ 10.40 (br s, 1H), 7.50 (dd, J = 2.8 Hz, J = 7.6 Hz, 1H), 7.45 (dt, J = 2.8 Hz, J = 8.7 Hz, 1H), 7.23-7.19 (m, 2H), 3.63 (s, 2H), 3.32 (s, 3H), 2.33-2.25 (m, 2H), 2.14-2.07 (m, 2H), 1.92-1.72 (m, 2H). |
| B | 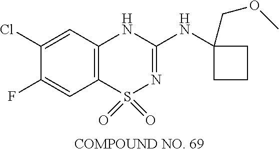 [SMILES: COCC1(NC2=NS(=O)(=O)c3cc(F)c(Cl)cc3N2)CCC1] | 6-chloro-7- fluoro-3-((1- (methoxymethyl) cyclobutyl) amino)-4H- benzo[e][1,2,4] thiadiazine 1,1-dioxide | 348.3 | ¹H-NMR (400 MHz, DMSO-d6): δ 10.29 (br s, 1H), 7.74 (d, J = 7.9 Hz, 1H), 7.53 (s, 1H), 7.41 (d, J = 6.1 Hz, 1H), 3.62 (s, 2H), 3.32 (s, 3H), 2.31-2.23 (m, 2H), 2.14-2.07 (m, 2H), 1.91-1.73 (m, 2H). |
| D | 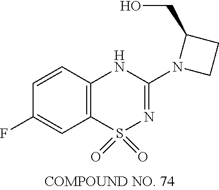 [SMILES: O=S1(=O)N=C(N2CC[C@@H]2CO)Nc2ccc(F)cc21] | (R)-7-fluoro- 3-(2- (hydroxymethyl) azetidin-1- yl)-4H- benzo[e][1,2,4] thiadiazine 1,1-dioxide | 286.4 | ¹H-NMR (400 MHz, DMSO-d6): δ 10.59 (br s, 1H), 7.51-7.43 (m, 2H), 7.32 (dd, J = 4.3 Hz, J = 8.9 Hz, 1H), 5.47 (br s, 1H), 4.57-4.50 (m, 1H), 4.04-3.94 (m, 2H), 3.82 (dd, J = 3.9 Hz, J = 11.5 Hz, 1H), 3.62 (dd, J = 5.0 Hz, J = 11.6 Hz, 1H), 2.37-2.29 (m, 1H), 2.21-2.12 (m, 1H). |
| D | 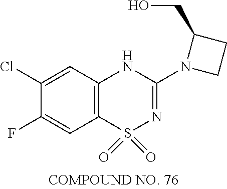 [SMILES: O=S1(=O)N=C(N2CC[C@@H]2CO)Nc2cc(Cl)c(F)cc21] | (R)-6-chloro- 7-fluoro-3-(2- (hydroxymethyl) azetidin-1- yl)-4H- benzo[e][1,2,4] thiadiazine 1,1-dioxide | 320.2 | ¹H-NMR (400 MHz, DMSO-d6): δ 10.56 (br s, 1H), 7.75 (d, J = 7.9 Hz, 1H), 7.49 (d, J = 6.1 Hz, 1H), 5.33 (br s, 1H), 4.57-4.49 (m, 1H), 4.04-3.95 (m, 2H), 3.82 (dd, J = 3.9 Hz, J = 11.5 Hz, 1H), 3.60 (dd, J = 4.6 Hz, J = 11.5 Hz, 1H), 2.38-2.29 (m, 1H), 2.22- 2.13 (m, 1H). |
| B | 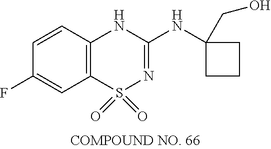 [SMILES: O=S1(=O)N=C(NC2(CO)CCC2)Nc2ccc(F)cc21] | 7-fluoro-3-((1- (hydroxymethyl) cyclobutyl) amino)-4H- benzo[e][1,2,4] thiadiazine 1,1-dioxide | 300.2 | ¹H-NMR (400 MHz, DMSO-d6): δ 10.43 (br s, 1H), 7.48 (dd, J = 2.8 Hz, J = 7.6 Hz, 1H), 7.44 (dt, J = 2.9 Hz, J = 8.8 Hz, 1H), 7.20-7.15 (m, 2H), 5.07 (br s, 1H), 3.63 (s, 2H), 2.30-2.21 (m, 2H), 2.12-2.06 (m, 2H), 1.89-1.67 (m, 2H). |
| B | 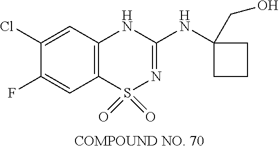 [SMILES: O=S1(=O)N=C(NC2(CO)CCC2)Nc2cc(Cl)c(F)cc21] | 6-chloro-7- fluoro-3-((1- (hydroxymethyl) cyclobutyl) amino)-4H- benzo[e][1,2,4] thiadiazine 1,1-dioxide | 334.2 | ¹H-NMR (400 MHz, DMSO-d6): δ 10.41 (br s, 1H), 7.74 (d, J = 7.9 Hz, 1H), 7.42 (d, J = 6.1 Hz, 1H), 7.37 (br s, 1H), 5.07 (br s, 1H), 3.64 (s, 2H), 2.28-2.20 (m, 2H), 2.13- 2.06 (m, 2H), 1.89-1.67 (m, 2H). |

### Example 5. Synthesis of Intermediate A2

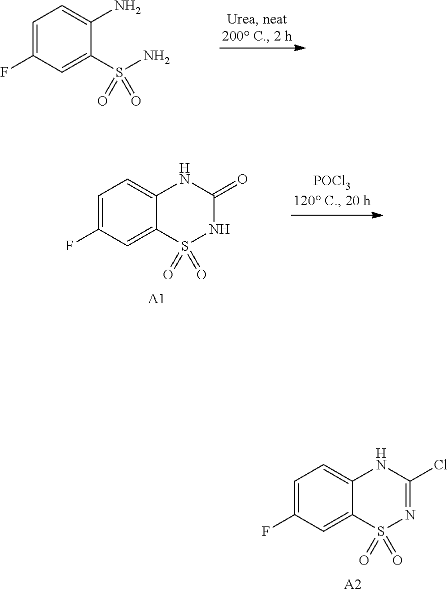

### Preparation of Intermediate 1 ((7-fluoro-2H-benzo[e][1,2,4]thiadiazin-3 (4H)-one 1,1-dioxide)

2-amino-5-fluorobenzene-1-sulfonamide (2.5 g, 13.1 mmol, 1 eq.) and urea (0.9 g, 14.5 mmol, 1.1 eq.) were mixed for 2 h at 200° C. Ammonia was evolved, and the mixture suddenly crystallized. The crude solid was dissolved in an aqueous solution of 1N NaOH (30 mL) and subsequently stirred for 5 min. The pH was adjusted to 1-2 with an aqueous solution of 1N HCl (40 mL), and the precipitate was collected by filtration, washed with water, and dried under vacuum to afford Intermediate A1 as a white solid (2.6 g, 83%). LC/MS (ESI+): m/z 217.1 [M+H]⁺. ¹H NMR (400 MHz, DMSO-d₆): δ 11.35 (s, 1H), 7.67 (dd, J=7.6, 2.8 Hz, 1H), 7.57-7.51 (m, 1H), 7.28 (dd, J=9.0, 4.4 Hz, 1H), and one NH missing.

### Preparation of Intermediate 2 (3-chloro-7-fluoro-4H-benzo[e][1,2,4]thiadiazine 1,1-dioxide)

A suspension of Intermediate A1 (2.4 g, 10.1 mmol, 1 eq.) in phosphorus oxychloride (14.1 mL, 151.5 mmol, 15 eq.) was stirred at 120° C. for 20 h. The mixture was then cooled to room temperature and poured into ice water (50 mL) and subsequently stirred for 30 min. An aqueous solution of 3N NaOH (350-400 mL) was then added dropwise at 0° C. until pH 9 over 30 min. The mixture was extracted with EtOAc (3×200 mL), the organic layers were combined, dried over anhydrous Na₂SO₄, filtered, and concentrated in vacuo to afford Intermediate A2 as a white powder (2.1 g). The product was used without further purification. LC/MS (ESI+): m/z 235.1 [M+H]⁺. ¹H NMR (400 MHz, DMSO-d₆): δ 7.38 (dd, J=7.8, 2.9 Hz, 1H), 7.36-7.30 (m, 1H), 7.19 (dd, J=9.0, 5.0 Hz, 1H), and one NH missing.

### Example 6. Synthesis of Intermediate 5

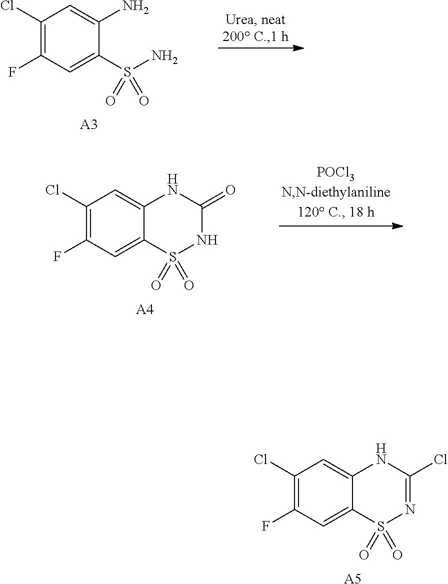

### Preparation of Intermediate 3 (2-amino-4-chloro-5-fluorobenzenesulfonamide)

Intermediate A3 was obtained according to the method previously described by P. De Tullio et al. (J. Med. Chem. 2005, 48, 4990-5000) starting from the corresponding 3-chloro-4-fluoroaniline.

### Preparation of Intermediate A4 (6-chloro-7-fluoro-2H-benzo[e][1,2,4]thiadiazin-3 (4H)-one 1,1-dioxide)

Intermediate A3 (3.0 g, 13.4 mmol, 1 eq.) and urea (0.9 g, 14.5 mmol, 1.1 eq.) were mixed for 1 h 200° C. Ammonia was evolved, and the mixture suddenly crystallized. The crude solid was then dissolved in an aqueous solution of 1N NaOH (30 mL) and stirred for 5 min. The pH was adjusted to 1-2 with an aqueous solution of 1N HCl (40 mL), and the precipitate was collected by filtration, washed with water, and dried under vacuum to afford Intermediate A4 as a purple solid (1.8 g). LC/MS (ESI+): m/z 251.1 [M+H]⁻. ¹H NMR (400 MHz, DMSO-d₆): δ 10.27 (s, 1H), 7.62 (d, J=7.9 Hz, 1H), 7.23 (d, J=6.1 Hz, 1H), and one NH missing.

### Preparation of Intermediate A5 (3,6-dichloro-7-fluoro-4H-benzo[e][1,2,4]thiadiazine 1,1-dioxide)

To a solution of Intermediate A4 (3.6 g, 14.4 mmol, 1 eq.) in phosphorus oxychloride (20.0 mL, 215 mmol, 15 eq.) was added N,N-diethylaniline (4.6 mL, 28.7 mmol, 2 eq.). The solution was heated to 120° C. for 18 h, then cooled to 25° C., poured into ice water (50 mL), and stirred for 1 h. The precipitate was collected by filtration, washed with water, and dried under vacuum. The crude residue was then purified by silica gel flash chromatography (Condition 1, Gradient 1) to give Intermediate A5 as a pale brown solid (2.63 g). LC/MS (ESI+): m/z 269.0 [M+H]⁺. ¹H NMR (400 MHz, DMSO-d₆): δ 8.05 (d, J=8.1 Hz, 1H), 7.50 (d, J=6.2 Hz, 1H), and one NH missing.

### Example 7. Synthesis of Intermediate A7

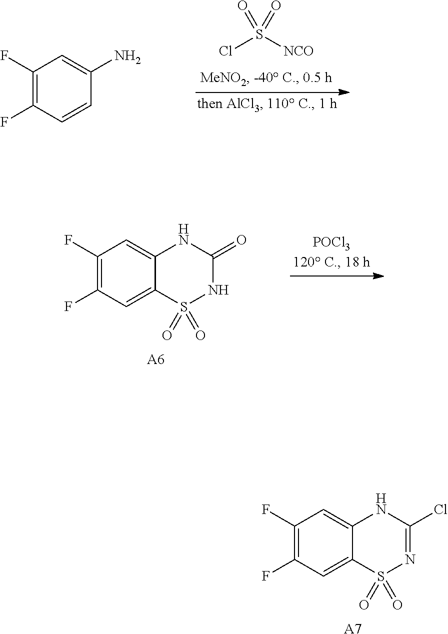

### Preparation of Intermediate A6 (6,7-difluoro-2H-benzo[e]f[1,2,4]thiadiazin-3 (4H)-one 1,1-dioxide)

To a solution of chlorosulfonyl isocyanate (0.809 mL, 9.29 mmol, 1.2 eq.) in nitromethane (7.4 mL) was added dropwise a solution of 3,4-difluoroaniline (0.768 mL, 7.75 mmol, 1 eq.) in nitromethane (2.3 mL) at −40° C. under argon atmosphere. The reaction mixture was stirred at −40° C. for 30 min and then allowed to warm to room temperature before the addition of aluminum chloride (1.34 g, 10.1 mmol, 1.3 eq.). The reaction mixture was stirred at 110° C. for 1 h and then cooled to 0° C. Water (25 mL) was added to the reaction mixture and the resulting precipitate was filtered, washed with water (2×10 mL) and dried under reduced pressure to afford Intermediate A6 as a brown solid (0.920 g, 51%). LC/MS (ESI−): m/z 233.0 [M−H]⁻. ¹H NMR (400 MHz, DMSO-d₆): δ 11.39 (s, 1H), 8.01 (dd, J=9.3, 8.0 Hz, 1H), 7.21 (dd, J=11.2, 6.6 Hz, 1H), and one NH missing.

### Preparation of Intermediate A7 (3-chloro-6,7-difluoro-4H-benzo[e][1,2,4]thiadiazine 1,1-dioxide)

A solution of Intermediate A6 (920 mg, 3.93 mmol, 1 eq.) in phosphorus oxychloride (9.15 mL, 98.2 mmol, 25 eq.) was stirred in a sealed vessel at 120° C. for 22 h. The mixture was cooled to room temperature, poured into ice water (100 mL) and stirred for 30 min. An aqueous solution of 10N NaOH was added dropwise at 0° C. until pH˜10. The mixture was saturated with solid NaCl, extracted with EtOAc (2×100 mL), and the combined organic layers were dried over Na₂SO₄, filtered, and concentrated under reduced pressure. The crude residue was purified by silica gel flash chromatography (Condition 1, Gradient 1) to afford Intermediate A7 as a pale yellow solid (566 mg). LC/MS (ESI+): m/z 253.0 [M+H]⁻. ¹H NMR (400 MHz, DMSO-d₆): δ 7.63 (dd, J 9.6, 8.6 Hz, 1H), 7.15 (dd, J 12.3, 7.3 Hz, 1H) and one NH missing.

### Example 8. Synthesis of Intermediates A9 and A9′

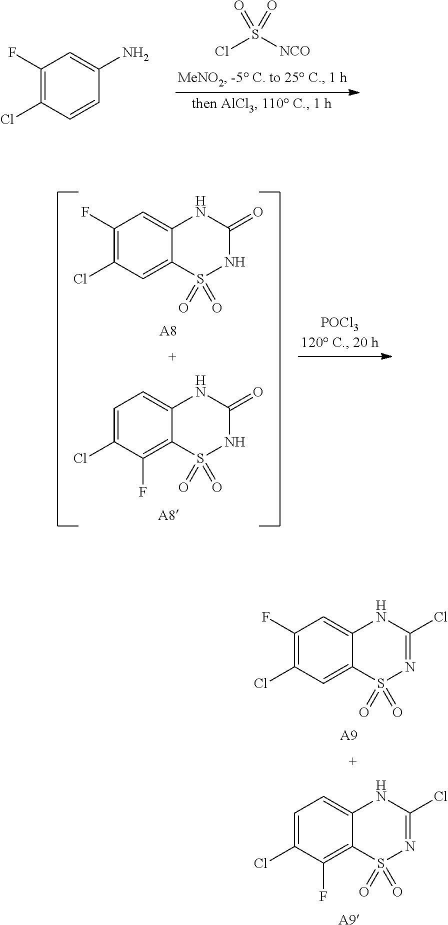

### Preparation of Intermediate A9 (3,7-dichloro-6-fluoro-4H-benzo[e][1,2,4]thiadiazine 1,1-dioxide) and Intermediate A9′ (3,7-dichloro-8-fluoro-4H-benzo[e][1,2,4]thiadiazine 1,1-dioxide)

To a solution of chlorosulfonyl isocyanate (0.941 mL, 10.8 mmol, 1.6 eq.) in nitromethane (9 mL) was added dropwise a solution of 4-chloro-3-fluoroaniline (1.00 g, 6.87 mmol, 1 eq.) in nitromethane (2.8 mL) at −5° C. under argon atmosphere. The reaction mixture was stirred from −5° C. to 10° C. for 1 h and then allowed to warm to room temperature. Aluminum chloride (1.56 g, 11.7 mmol, 1.7 eq.) was then added and the reaction mixture was stirred at 110° C. for 1 h. The reaction mixture was subsequently cooled to room temperature, poured into ice water (˜50 mL) and finally stirred for 30 min. The resulting precipitate was filtered, washed with water (2×15 mL) and dried under reduced pressure to afford a mixture of Intermediates A8 and A8′ as a grey solid (1.02 g). The mixture of Intermediates A8 and A8′ was directly used as such in the next step without purification.

A suspension of Intermediates A8 and A8′ (1.00 g, 3.99 mmol, 1 eq.) in phosphorus oxychloride (7.44 mL, 79.8 mmol, 20 eq.) was stirred in a sealed vessel at 120° C. for 20 h. The mixture was then cooled to room temperature, poured into ice water (˜100 mL) and stirred for 30 min. An aqueous solution of 3N NaOH (250 mL) was then added at 0° C. until pH˜10 and the resulting mixture was saturated with solid NaCl. The mixture was extracted with EtOAc (3×250 mL), the combined organic layers were dried over Na₂SO₄, filtered, and concentrated under reduced pressure. The crude residue was purified by silica gel flash chromatography (Condition 1, Gradient 1, then Condition 2, Gradient 1) to afford an inseparable mixture of Intermediates A9 and A9′ as a beige solid (525 mg). ¹H NMR ratio A9/A9′ (DMSO-d₆)=80:20). LC/MS (ESI−): m/z 266.9 [M−H]⁻. ¹H NMR (400 MHz, DMSO-d₆) of mixture of Intermediates A9 and A9′: δ 7.76 (d, J=8.0 Hz, 0.8H, Intermediate A9), 7.58 (t, J=8.6 Hz, 0.2H, Intermediate A9′), 7.12 (d, J=11.0 Hz, 0.8H, Intermediate A9), 6.99 (dd, J=8.9, 1.5 Hz, 0.2H, Intermediate A9′) and both NH are missing.

### Example 9. Synthesis of Intermediates A11 and A11′

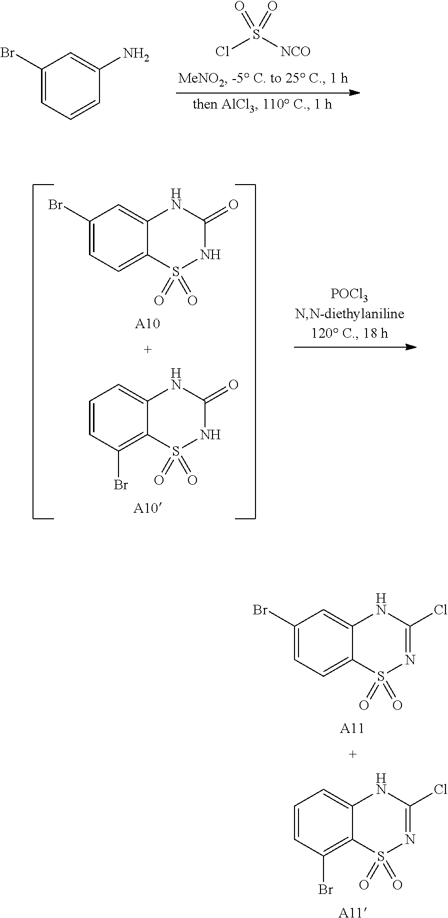

### Preparation of Intermediate A11 (6-bromo-3-chloro-4H-benzo[e][1,2,4]thiadiazine 1,1-dioxide) & Intermediate A11′ (8-bromo-3-chloro-4H-benzo[e][1,2,4]thiadiazine 1,1-dioxide)

To a solution of chlorosulfonyl isocyanate (0.607 mL, 6.98 mmol, 1.2 eq.) in nitromethane (5.8 mL) was added dropwise a solution of 3-bromoaniline (0.633 mL, 5.81 mmol, 1 eq.) in nitromethane (1.8 mL) at −5° C. under argon atmosphere. The reaction mixture was stirred from −5° C. to 10° C. for 1 h and then allowed to warm to room temperature. Aluminum chloride (1.01 g, 7.56 mmol, 1.3 eq.) was added and the reaction mixture was stirred at 110° C. for 1 h. The reaction mixture was then cooled to room temperature, poured into ice water (˜50 mL) and stirred for 30 min. The resulting precipitate was filtered, washed with water (2×10 mL) and dried under reduced pressure to afford a mixture of Intermediates A10 and A10′ as a grey solid (0.540 g). The mixture of Intermediates A10 and A10′ was directly used as such in the next step without purification.

To a suspension of Intermediates A10 and A10′ (530 mg, 1.91 mmol, 1 eq.) in phosphorus oxychloride (4.46 mL, 47.8 mmol, 25 eq.) was added N,N-diethylaniline (1.01 mL, 6.31 mmol, 3.3 eq.) at room temperature. The reaction mixture was stirred in a sealed vessel at 100° C. for 18 h and then at 120° C. for 3 h. The reaction mixture was subsequently cooled to room temperature, poured into ice water (˜150 mL) and stirred for 30 min. An aqueous solution of 3N NaOH (100 mL) was added at 0° C. until pH˜8, and the resulting mixture was stirred for 1 h. The mixture was then re-acidified with an aqueous solution of 1N HCl (60 mL) until pH˜2, extracted with EtOAc (2×100 mL), and the combined organic layers were dried over Na₂SO₄, filtered, and concentrated under reduced pressure. The crude residue was purified by silica gel flash chromatography (Condition 1, Gradient 1) followed by preparative HPLC (Condition 1, Gradient 1) to afford Intermediate A11 (171 mg) and Intermediate A11′ (18 mg) as white solids. Analytical data for Intermediate A11: LC/MS (ESI+): m/z 295.1 [M+H]⁺. ¹H NMR (400 MHz, DMSO-d₆): δ 7.82 (d, J=8.5 Hz, 1H), 7.68 (dd, J=8.5, 1.9 Hz, 1H), 7.51 (d, J=1.8 Hz, 1H) and one NH missing. Analytical data for Intermediate A11′: LC/MS (ESI+): m/z 295.1 [M+H]⁺. ¹H NMR (400 MHz, DMSO-d₆): δ 7.73 (dd, J=7.9, 1.1 Hz, 1H), 7.60 (t, J=8.1 Hz, 1H), 7.38 (dd, J=8.3, 1.1 Hz, 1H) and one NH missing.

### Example 10. Synthesis of Intermediates A13 and A13′

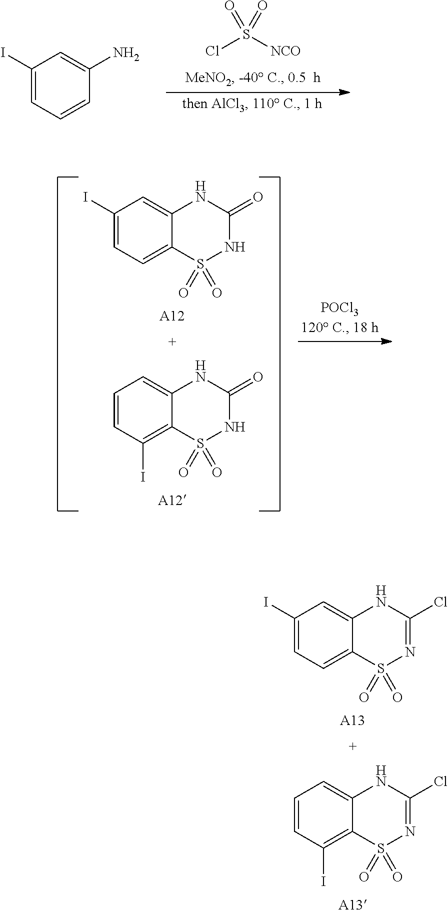

### Preparation of Intermediate A13 (3-chloro-6-iodo-4H-benzo[e][1,2,4]thiadiazine 1,1-dioxide) & Intermediate 13′ (3-chloro-6-iodo-4H-benzo[e][1,2,4]thiadiazine 1,1-dioxide)

To a solution of chlorosulfonyl isocyanate (2.38 mL, 27.4 mmol, 1.2 eq.) in nitromethane (21.7 mL) was added dropwise a solution of 3-iodoaniline (2.75 mL, 22.8 mmol, 1 eq.) in nitromethane (6.8 mL) at −40° C. under argon atmosphere. The reaction mixture was stirred at −40° C. for 30 min and then allowed to warm to room temperature before the addition of aluminum chloride (3.96 g, 29.7 mmol, 1.3 eq.). The reaction mixture was stirred at 110° C. for 1 h and then cooled to 0° C. Water (50 mL) was then added and the reaction mixture was stirred for 30 min. The resulting precipitate was filtered, washed with water (2×20 mL) and dried under reduced pressure to afford a mixture of Intermediates A12 and A12′ as a brown solid (3.64 g). The mixture of Intermediates A12 and A12′ was directly used as such in the next step without purification.

A suspension of Intermediates A12 and A12′ (250 mg, 0.77 mmol, 1 eq.) in phosphorus oxychloride (1.44 mL, 15.4 mmol, 20 eq.) was stirred in a sealed vessel at 120° C. for 18 h. The mixture was then cooled to room temperature, poured into ice water (˜50 mL) and stirred for 30 min. An aqueous solution of 3N NaOH (50 mL) was added at 0° C. until pH˜8 and the resulting mixture was stirred for 1 h. The mixture was re-acidified with an aqueous solution of 1N HCl (30 mL) until pH˜2 and then saturated with solid NaCl. The aqueous layer was extracted with EtOAc (3×100 mL), and the combined organic layers were dried over Na₂SO₄, filtered, and concentrated under reduced pressure. The crude residue was purified by silica gel flash chromatography (Condition 1, Gradient 3) to afford Intermediate A13 (75.0 mg) and Intermediate A13′ (49 mg) as brown solids. Analytical data for Intermediate A13: LC/MS (ESI−): m/z 340.9 [M−H]⁻. ¹H NMR (400 MHz, CD₃OD) δ 7.86 (dd, J=8.4, 1.5 Hz, 1H), 7.68 (d, J=1.5 Hz, 1H), 7.61 (d, J=8.4 Hz, 1H) and one NH missing. Analytical data for Intermediate A13′: LC/MS (ESI−): m/z 340.9 [M−H]⁻. ¹H NMR (400 MHz, CD₃OD): δ 8.07 (dd, J=7.4, 1.4 Hz, 1H), 7.40 (dd, J=8.3, 7.4 Hz, 1H), 7.35 (dd, J=8.3, 1.4 Hz, 1H) and one NH missing.

### Example 11. Synthesis of Compound 1 From Intermediate A2

A mixture of Intermediate A2 (1.00 g, 4.26 mmol, 1 eq.), bicyclo[1.1.1]pentan-1-amine hydrochloride (0.815 g, 6.82 mmol, 1.6 eq.) and DIEA (1.93 mL, 11.1 mmol, 2.6 eq.) in dioxane (36 mL), was heated in a sealed vessel at 100° C. for 2 h. The reaction mixture was concentrated under reduced pressure and then partitioned between EtOAc (100 mL) and a water/brine mixture (2:1, 40 mL). The layers were separated and the organic layer was washed with a water/brine mixture (2:1, 2×40 mL), dried over Na₂SO₄, filtered and concentrated under reduced pressure. The crude residue was purified by flash chromatography over silica gel (Condition 1, Gradient 1) to afford Compound 1 as a white solid (0.780 g). LC/MS (ESI+): m/z 282.1 [M+H]⁺; Rt=6.99 min. ¹H NMR (400 MHz, DMSO-d₆): δ 10.44 (s, 1H), 7.80 (s, 1H), 7.50 (dd, J=7.6, 2.9 Hz, 1H), 7.45 (dt, J=8.8, 2.9 Hz, 1H), 7.25 (dd, J=8.8, 4.4 Hz, 1H), 2.47 (s, 1H), 2.08 (s, 6H).

LC/MS (ESI+): m/z X [M+H]⁺; Rt=Y min.

The compounds reported in the following Table 3 were prepared via an analogous procedure from Intermediate A2 as a starting material.

TABLE 3

| Compound | | | |
|---|---|---|---|
| No. | Structure | Name | Analytical Data |
| 2 | 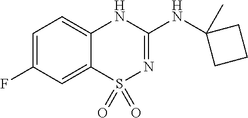 [SMILES: CC1(NC2=NS(=O)(=O)c3cc(F)ccc3N2)CCC1] | 7-fluoro-3-((1- methylcyclobutyl)amino)- 4H- benzo[e][1,2,4]thiadiazine 1,1-dioxide | LC/MS (ESI+): m/z 284.2 [M + H]⁺; Rt = 5.35 min. ¹HNMR (400 MHz, DMSO-d₆): δ 10.34 (s, 1H), 7.48 (dd, J = 7.6, 3.0 Hz, 1H), 7.44 (dt, J = 8.7, 2.8 Hz, 1H), 7.23 (s, 1H), 7.21 (dd, J = 8.9, 4.3 Hz, 1H), 2.31-2.23 (m, 2H), 2.04-1.98 (m, 2H), 1.85-1.77 (m, 2H), 1.49 (s, 3H). |
| 3 | 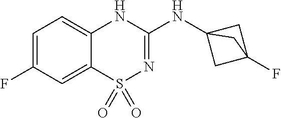 [SMILES: O=S1(=O)N=C(NC23CC(F)(C2)C3)Nc2ccc(F)cc21] | 7-fluoro-3-((3- fluorobicyclo[1.1.1]pentan- 1-yl)amino)-4H- benzo[e][1,2,4]thiadiazine 1,1-dioxide | LC/MS (ESI+): m/z 300.1 [M + H]⁺; Rt = 5.25 min. ¹HNMR (400 MHz, DMSO-d₆): δ 10.71 (br s, 1H). 8.02 (s, 1H), 7.53 (dd, J = 7.6, 2.9 Hz, 1H), 7.46 (dt, J = 8.7, 2.9 Hz. 1H), 7.28 (dd, J = 9.0, 4.4 Hz, 1H), 2.42 (d, J = 2.2 Hz, 6H). |
| 4 | 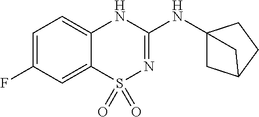 [SMILES: O=S1(=O)N=C(NC23CCC(C2)C3)Nc2ccc(F)cc21] | 3-(bicyclo[2.1.1]hexan-1- ylamino)-7-fluoro-4H- benzo[e][1,2,4]thiadiazine 1,1-dioxide | LC/MS (ESI+): m/z 296.2 [M + H]⁺; Rt = 5.33 min. ¹HNMR (400 MHz, DMSO-d₆): δ 10.37 (s, 1H), 7.61 (s, 1H), 7.50 (dd, J = 7.6, 2.9 Hz, 1H), 7.45 (dt, J = 8.8, 2.9 Hz. 1H), 7.23 (dd, J = 9.0. 4.3 Hz, 1H), 2.35-2.32 (m, 1H), 1.89-1.85 (m, 2H), 1.84-1.78 (m, 2H), 1.67-1.63 (m, 2H), 1.47-1.41 (m, 2H). |
| 5 | 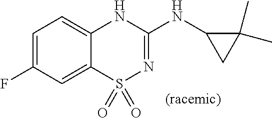 [SMILES: CC1(C)CC1NC1=NS(=O)(=O)c2cc(F)ccc2N1] | 3-((2,2- dimethylcyclopropyl) amino)- 7-fluoro-4H- benzo[e][1,2,4]thiadiazine 1,1-dioxide | LC/MS (ESI+): m/z 284.2 [M + H]⁺; Rt = 5.09 min. ¹HNMR (400 MHz, DMSO-d₆): δ 10.51 (s, 1H), 7.72-7.35 (m, 3H), 7.31 (s, 1H), 2.47-2.42 (m, 1H), 1.09 (s, 3H), 1.02 (s, 3H), 0.76 (dd, J = 7.8, 5.2 Hz. 1H), 0.43 (t, J = 4.6 Hz, 1H). |
| 6 | 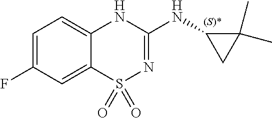 [SMILES: CC1(C)C[C@@H]1NC1=NS(=O)(=O)c2cc(F)ccc2N1] | (S*)-3-((2,2- dimethylcyclopropyl) amino)-7-fluoro-4H- benzo[e][1,2,4]thiadiazine 1,1-dioxidc | LC/MS (ESI+): m/z 284.4 [M + H]⁺; Rt = 5.05 min. ¹HNMR (400 MHz, DMSO-d₆): δ 10.51 (br s, 1H), 7.74-7.37 (m, 3H), 7.31 (br s, 1H), 2.47-2.42 (m, 1H), 1.09 (s, 3H), 1.02 (s, 3H), 0.76 (dd, J = 7.7, 5.2 Hz, 1H), 0.43 (t, J = 4.7 Hz, 1H). |
| | | | SFC: (Separation: Gradient: Condition 3, |
| | | | Gradient 1; Analytical: Condition 1, Gradient 1) |
| | | | Rt = 1.16 min; purity = 100%. |
| | | | Absolute stereochemistry was assigned arbitrarily. |
| 7 | 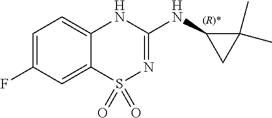 [SMILES: CC1(C)C[C@H]1NC1=NS(=O)(=O)c2cc(F)ccc2N1] | (R*)-3-((2,2- dimethylcyclopropyl) amino)- 7-fluoro-4H- benzo[e][1,2,4]thiadiazine 1,1-dioxide | LC/MS (ESI+): m/z 284.4 [M + H]⁺; Rt = 5.06 min. ¹HNMR (400 MHz, DMSO-d₆): δ 10.55 (br s, 1H), 7.74-7.37 (m, 3H), 7.31 (br s, 1H), 2.47-2.42 (m, 1H), 1.09 (s, 3H), 1.02 (s, 3H), 0.76 (dd, J = 7.7, 5.2 Hz, 1H), 0.43 (t, J = 4.7 Hz, 1H). |
| | | | SFC: (Separation: Gradient: Condition 3. |
| | | | Gradient 1; Analytical: Condition 1, Gradient 1) |
| | | | Rt = 2.47 min; purity = 100%. |
| | | | Absolute stereochemistry was assigned arbitrarily. |
| 8 | 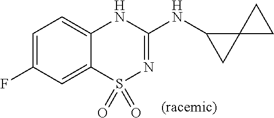 [SMILES: O=S1(=O)N=C(NC2CC23CC3)Nc2ccc(F)cc21] | 7-fluoro-(spiro[2.2]pentan- 1-ylamino)-4H- benzo[e][1,2,4]thiadiazine 1,1-dioxide | LC/MS (ESI+): m/z 282.2 [M + H]⁺; Rt = 4.94 min. ¹H NMR (400 MHz, DMSO-d₆): δ 10.51 (s, 1H), 7.57 (br s, 1H), 7.51-7.42 (m, 2H), 7.32 (s, 1H), 3.00-2.96 (m, 1H), 1.32-1.29 (m, 1H), 1.02-0.93 (m, 3H), 0.87-0.83 (m, 1H), 0.79-0.74 (m, 1H). |
| 9 | 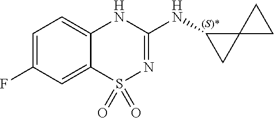 [SMILES: O=S1(=O)N=C(N[C@H]2CC23CC3)Nc2ccc(F)cc21] | (S*)-7-fluoro-3- (spiro[2.2]pentan-1- ylamino)-4H- benzo[e][1,2,4]thiadiazine 1,1-dioxide | LC/MS (ESI+): m/z 282.4 [M + H]⁺; Rt = 4.93 min. ¹HNMR (400 MHz, DMSO-d₆): δ 10.32 (br s, 1H), 7.62 (br s, 1H), 7.52-7.40 (m, 2H), 7.31 (br s, 1H). 3.00-2.95 (m, 1H), 1.32-1.29 (m, 1H), 1.03-0.93 (m, 3H), 0.87-0.83 (m, 1H), 0.80-0.74 (m, 1H). |
| | | | SFC: (Separation:Condition 2, Gradient 1; |
| | | | Analytical: Condition 2, Gradient 1) |
| | | | Rt = 1.74 min; purity = 100%. |
| | | | Absolute stereochemistry was assigned arbitrarily. |
| 10 | 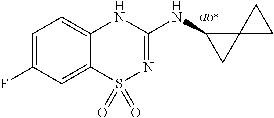 [SMILES: O=S1(=O)N=C(N[C@@H]2CC23CC3)Nc2ccc(F)cc21] | (R*)-7-fluoro-3- (spiro[2.2]pentan-1- ylamino)-4H- benzo[e][1,2,4]thiadiazine 1,1-dioxide | LC/MS (ESI+): m/z 282.4 [M + H]⁺; Rt = 4.93 min. ¹HNMR (400 MHz, DMSO-d₆): δ 10.20 (br s, 1H), 7.67 (br s. 1H), 7.52-7.40 (m, 2H), 7.31 (br s, 1H), 3.00-2.95 (m, 1H), 1.32-1.29 (m, 1H), 1.03-0.93 (m, 3H), 0.87-0.83 (m, 1H), 0.80-0.74 (m, 1H). |
| | | | SFC: (Separation: Condition 2,Gradient 1; |
| | | | Analytical: Condition 2, Gradient 1) |
| | | | Rt = 2.93 min; purity = 99.5%. |
| | | | Absolute stereochemistry was assigned arbitrarily. |
| 11 | 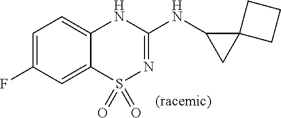 [SMILES: O=S1(=O)N=C(NC2CC23CCC3)Nc2ccc(F)cc21] | 7-fluoro-3(spiro[2.3]hexan- 1-ylamino)-4H- benzo[e][1,2,4]thiadiazine 1,1-dioxide | LC/MS (ESI+): m/z 296.4 [M + H]⁺; Rt = 7.51 min. ¹HNMR (400 MHz, DMSO-d₆): δ 10.57 (s, 1H). 7.51 (dd, J = 7.6, 2.9 Hz, 1H), 7.47 (dt, J = 8.7, 2.9 Hz, 1H), 7.34 (br s, 2H), 2.56-2.52 (m, 1H), 2.18-2.06 (m, 3H), 2.03-1.91 (m, 3H), 0.93-0.90 (m, 1H), 0.56-0.54 (m, 1H). |
| 12 | 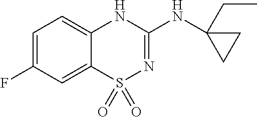 [SMILES: CCC1(NC2=NS(=O)(=O)c3cc(F)ccc3N2)CC1] | 3-((1- ethylcyclopropyl)amino)-7- fluoro-4H- benzo[e][1,2,4]thiadiazine 1,1-dioxide | LC/MS (ESI+): m/z 284.5 [M + H]⁺; Rt = 4.99 min. ¹H NMR (400 MHz, DMSO-d₆): δ 10.26 (br s, 1H), 7.62 (br s, 1H). 7.49- 7.42 (m, 2H), 7.35 (br s, 1H), 1.61 (q, J = 7.4 Hz, 2H), 0.91 (t, J = 7.4 Hz, 3H), 0.76-0.68 (m, 4H). |
| 13 | 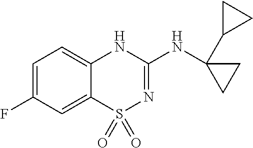 [SMILES: O=S1(=O)N=C(NC2(C3CC3)CC2)Nc2ccc(F)cc21] | 3-([1,1-bi(cyclopropan)]1- ylamino)-7-fluoro-4H- benzo[e][1,2,4]thiadiazine 1,1-dioxide | LC/MS (ESI+): m/z 296.4 [M + H]⁺; Rt = 5.10 min. ¹H NMR (400 MHz, DMSO-d₆): δ 10.32 (s, 1H), 7.72 (br s, 1H), 7.50-7.43 (m, 2H), 7.37 (br s, 1H), 1.46-1.39 (m, 1H), 0.72-0.69 (m, 2H), 0.66-0.63 (m, 2H), 0.42-0.37 (m, 2H), 0.22-0.18 (m, 2H). |

### Example 12. Synthesis of Compound 14 From Intermediate A5

A mixture of Intermediate 5 (0.410 g, 1.52 mmol, 1 eq.), bicyclo[1.1.1]pentan-1-amine hydrochloride (0.292 g, 2.44 mmol, 1.6 eq.) and DIEA (0.690 mL, 3.96 mmol, 2.6 eq.) in dioxane (10 mL) was heated in a sealed vessel at 100° C. for 2 h. The reaction mixture was concentrated under reduced pressure and then partitioned between EtOAc (80 mL) and a water/brine mixture (2:1, 20 mL). The layers were separated and the organic layer was washed with a water/brine mixture (2:1, 2×20 mL), dried over Na₂SO₄, filtered and concentrated under reduced pressure. The crude residue was purified by flash chromatography over silica gel (Condition 1, Gradient 2) The resulting solid was triturated in EtOH (5 mL), filtered and dried under reduced pressure to afford Compound 14 as a white solid (0.269 g). LC/MS (ESI+): m/z 316.2 [M+H]⁺; Rt=5.74 min.

¹H NMR (400 MHz, DMSO-d₆): δ 10.44 (s, 1H), 8.05 (s, 1H), 7.77 (d, J=7.9 Hz, 1H), 7.46 (d, J=6.1 Hz, 1H), 2.48 (s, 1H), 2.09 (s, 6H).

The compounds reported in the following Table 4 were prepared via an analogous procedure.

TABLE 4

| Compound | | | Analytical |
|---|---|---|---|
| No. | Structure | Name | Data |
| 15 | 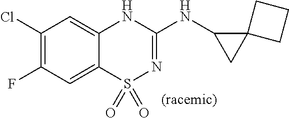 [SMILES: O=S1(=O)N=C(NC2CC23CCC3)Nc2cc(Cl)c(F)cc21] | 6-chloro-7-fluoro-3- (spiro[2.3]hexan-1-ylamino)-4H- benzo[e][1,2,4]thiadiazine 1,1- dioxide | LC/MS (ESI+): m/z 330.1 [M + H]⁺; Rt = 8.34 min. ¹H NMR (400 MHz, DMSO-d₆): δ 10.57 (s, 1H), 7.78 (d, J = 7.9 Hz, 1H), 7.53 (br s, 2H), 2.58-2.54 (m, 1H), 2.17-2.05 (m, 3H), 2.01-1.94 (m, 3H), 0.94-0.91 (m, 1H), 0.58-0.54 (m, 1H). |
| 16 | 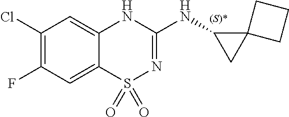 [SMILES: O=S1(=O)N=C(N[C@H]2CC23CCC3)Nc2cc(Cl)c(F)cc21] | (S*)-6-chloro-7-fluoro-3- (spiro[2.3]hexan-1-ylamino)-4H- benzo[e][1,2,4]-thiadiazine 1,1- dioxide | LC/MS (ESI+): m/z 330.1 [M + H]⁺; Rt = 5.93 min. ¹H NMR (400 MHz, DMSO-d₆): δ 10.57 (s, 1H), 7.78 (d, J = 7.9 Hz, 1H), 7.53 (br s, 2H), 2.58-2.54 (m, 1H), 2.17-2.05 (m, 3H), 2.01-1.94 (m, 3H), 0.95-0.89 (m, 1H), 0.59-0.53 (m, 1H). |
| | | | SFC: Separation: Condition 2, |
| | | | Gradient 2; Analytical: Condition 3, |
| | | | Gradient 1) Rt = 1.48 min; purity = |
| | | | 97.4%. Absolute stereochemistry was |
| | | | assigned arbitrarily. |
| 17 | 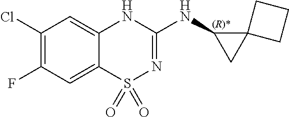 [SMILES: O=S1(=O)N=C(N[C@@H]2CC23CCC3)Nc2cc(Cl)c(F)cc21] | (R*)-6-chloro-7-fluoro-3- (spiro[2.3]hexan-1-ylamino)-4H- benzo[e][1,2,4]-thiadiazine 1,1- dioxide | LC/MS (ESI+): m/z 330.1 [M + H]⁺; Rt = 5.92 min. ¹H NMR (400 MHz, DMSO-d₆): δ 10.57 (s, 1H), 7.78 (d, J = 7.9 Hz, 1H), 7.53 (br s, 2H), 2.58-2.54 (m, 1H), 2.17- 2.05 (m, 3H), 2.01-1.94 (m, 3H), 0.95-0.89 (m, 1H), 0.59 0.53 (m, 1H). SFC: (Separation: Condition 2, |
| | | | Gradient 1; Analytical: Condition 2, |
| | | | Gradient 1) Rt = 4.38 min; purity = 100%. |
| | | | Absolute stereochemistry was assigned |
| | | | arbitrarily. |
| 18 | 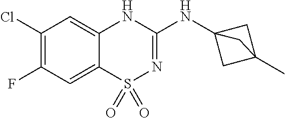 [SMILES: CC12CC(NC3=NS(=O)(=O)c4cc(F)c(Cl)cc4N3)(C1)C2] | 6-chloro-7-fluoro-3-((3- methylbicyclo[1.1.1]pentan- 1-yl)amino)-4H- benzo[e][1,2,4]thiadiazine 1,1-dioxide | LC/MS (ESI+): m/z 330.1 [M + H]⁺; Rt = 8.29 min. ¹H NMR (400 MHz, DMSO-d₆): δ 10.40 (s, 1H), 7.99 (s, 1H), 7.76 (d, J = 7.9 Hz, 1H), 7.46 (d, J = 6.1 Hz, 1H), 1.95 (s, 6H), 1.23 (s, 3H). |
| 19 | 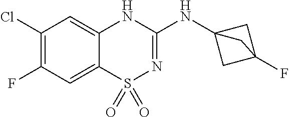 [SMILES: O=S1(=O)N=C(NC23CC(F)(C2)C3)Nc2cc(Cl)c(F)cc21] | 6-chloro-7-fluoro-3-((3- fluorobicyclo[1.1.1 ]pentan-1- yl)amino)-4H- benzo[e][1,2,4]thiadiazine 1,1-dioxide | LC/MS (ESI+): m/z 334.1 [M + H]⁺; Rt = 7.79 min. ¹H NMR (400 MHz, DMSO-d₆): δ 10.69 (s, 1H), 8.25 (s, 1H), 7.80 (d, J = 7.9 Hz, 1H), 7.48 (d, J = 6.1 Hz, 1H), 2.42 (d. J = 2.2 Hz, 6H). |
| 20 | 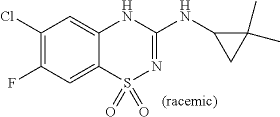 [SMILES: CC1(C)CC1NC1=NS(=O)(=O)c2cc(F)c(Cl)cc2N1] | 6-chloro-3-((2,2- dimethylcyclopropyl)amino)- 7-fluoro-4H-benzo[e][1,2,4] thiadiazine 1,1-dioxide | LC/MS (ESI+): m/z 318.1 [M + H]⁺; Rt = 5.75 min. ¹H NMR (400 MHz, DMSO-d₆): δ 10.53 (s. 1H), 7.77 (d, J = 8.0 Hz, 1H), 7.52 (br s, 2H), 2.47-2.42 (m, 1H), 1.09 (s, 3H), 1.01 (s, 3H), 0.78-0.75 (m, 1H), 0.46-0.43 (m, 1H). |
| 21 | 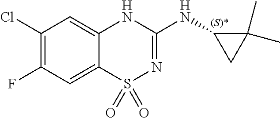 [SMILES: CC1(C)C[C@@H]1NC1=NS(=O)(=O)c2cc(F)c(Cl)cc2N1] | (S*)-6-chloro-3-((2,2- dimethylcyclopropyl)amino)- 7-fluoro-4H benzo[e][1,2,4] thiadiazine 1,1-dioxide | LC/MS (ESI+): m/z 318.2 [M + H]⁺; Rt = 5.84 min. ¹H NMR (400 MHz, DMSO-d₆): δ 10.53 (s, 1H), 7.76 (d, J = 8.0 Hz, 1H), 7.52 (br s, 2H), 2.47-2.42 (m, 1H), 1.09 (s, 3H), 1.02 (s, 3H), 0.78-0.75 (m, 1H), 0.46-0.43 (m, 1H). SFC: (Separation: Condition 3, |
| | | | Gradient 1; Analytical: Condition 1, |
| | | | Gradient 2) Rt = 1.12 min; |
| | | | purity = 100%. Absolute stereochemistry |
| | | | was assigned arbitrarily. |
| 22 | 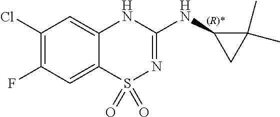 [SMILES: CC1(C)C[C@H]1NC1=NS(=O)(=O)c2cc(F)c(Cl)cc2N1] | (R*)-6-chloro-3-((2,2- dimethylcyclopropyl)amino)- 7-fluoro-4H-benzo[e][1,2,4] thiadiazine 1,1-dioxide | LC/MS (ESI+): m/z 318.2 [M + H]⁺; Rt = 5.83 min. ¹H NMR (400 MHz, DMSO-d₆): δ 10.55 (s, 1H), 7.76 (d, J = 8.0 Hz, 1H), 7.51 (br s, 2H), 2.47-2.42 (m, 1H), 1.09 (s, 3H), 1.02 (s, 3H), 0.78- 0.75 (m, 1H), 0.46-0.43 (m, 1H). |
| | | | SFC: Rt = 2.57 min; purity = 100%. |
| | | | Absolute stereochemistrywas assigned |
| | | | arbitrarily. (Separation: Condition 3, |
| | | | Gradient 1; Analytical: Condition 1, |
| | | | Gradient 2) |
| 23 | 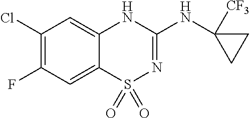 [SMILES: O=S1(=O)N=C(NC2(C(F)(F)F)CC2)Nc2cc(Cl)c(F)cc21] | 6-chloro-7-fluoro-3-((1- (trifluoromethyl)cyclopropyl) amino)-4H-benzo[e][1,2,4] thiadiazine 1,1-dioxide | LC/MS (ESI+): m/z 358.1 [M + H]⁺; Rt = 7.83 min. ¹H NMR (400 MHz, DMSO-d₆): δ 10.65 (br s, 1H), 8.41 (br s, 1H), 7.81 (d, J = 8.0 Hz, 1H), 7.62 (br s, 1H), 1.42-1.38 (m, 2H), 1.28-1.24 (m, 2H). |
| 24 | 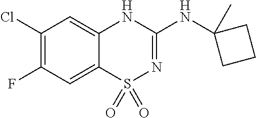 [SMILES: CC1(NC2=NS(=O)(=O)c3cc(F)c(Cl)cc3N2)CCC1] | 6-chloro-7-fluoro-3-((1- methylcyclobutyl)amino)-4H- benzo[e][1,2,4]thiadiazine 1,1-dioxide | LC/MS (ESI+): m/z 318.1 [M + H]⁺; Rt = 7.95 min. ¹H NMR (400 MHz, DMSO-d₆): δ 10.33 (s, 1H), 7.75 (d. J = 8.0 Hz, 1H), 7.48 (s, 1H), 7.42 (d, J = 6.0 Hz, 1H), 2.31-2.23 (m, 2H), 2.05-1.99 (m, 2H), 1.86-1.78 (m, 2H), 1.49 (s, 3H). |
| 25 | 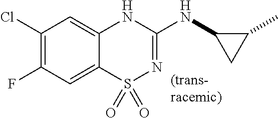 | (rac)-trans-6-chloro-7- fluoro-3-((2-methylcyclopropyl)amino)-4H- benzo[e][1,2,4\|thiadiazine 1,1-dioxide | LC/MS (ESI+): m/z 304.1 [M + H]⁺; Rt = 5.53 min. ¹H NMR (400 MHz, DMSO-d₆): δ 10.48 (br s, 1H), 7.84 (br s, 1H), 7.76 (d, J = 8.0 Hz, 1H), 7.57 (br s, 1H), 2.40 2.36 (m, 1H), 1.07 (d. J = 6.1 Hz, 3H), 0.99-0.91 (m, 1H), 0.73-0.69 (m, 1H), 0.61-0.56 (m, 1H). |
| 26 | 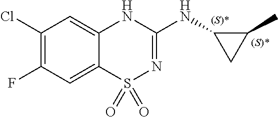 [SMILES: C[C@H]1C[C@@H]1NC1=NS(=O)(=O)c2cc(F)c(Cl)cc2N1] | 6-chloro-7-fluoro-3- (((1S*, 2S*)-2- methylcyclopropyl)amino)-4H- benzo[e][1,2,4]-thiadiazine 1,1-dioxide | LC/MS (ESI+): m/z 304.2 [M + H]⁺; Rt = 5.56 min. ¹H NMR (400 MHz, DMSO-d₆): δ 10.49 (br s, 1H), 7.82 (br s, 1H), 7.75 (d, J = 7.9 Hz, 1H), 7.55 (br s, 1H), 2.40-2.36 (m, 1H), 1.07 (d, J = 6.1 Hz, 3H), 0.99-0.91 (m, 1H), 0.73-0.69 (m, 1H), 0.60-0.56 (m, 1H). |
| | | | SFC: (Separation: Condition 3, |
| | | | Gradient 1; Analytical: Condition 1, |
| | | | Gradient 2) Rt = 1.61 min; purity = 100%. |
| | | | Absolute stereochemistry was assigned |
| | | | arbitrarily. |
| 27 | 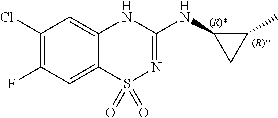 [SMILES: C[C@@H]1C[C@H]1NC1=NS(=O)(=O)c2cc(F)c(Cl)cc2N1] | 6-chloro-7-fluoro-3- (((1R*, 2R*)-2 methylcyclopropyl)amino)-4H- benzo[e][1,2,4]-thiadiazine 1,1-dioxide | LC/MS (ESI+): m/z 304.2 [M + H]⁺; Rt = 5.58 min. ¹H NMR (400 MHz, DMSO-d₆): δ 10.50 (br s, 1H), 7.82 (br s, 1H), 7.75 (d, J = 7.9 Hz, 1H), 7.56 (br s, 1H), 2.40- 2.36 (m, 1H), 1.07 (d, J = 6.0 Hz. 3H), 0.99-0.91 (m, 1H), 0.73-0.69 (m, 1H), 0.60-0.56 (m, 1H). |
| | | | SFC: (Separation: Condition 3, |
| | | | Gradient 1; Analytical: Condition 1, |
| | | | Gradient 2) Rt = 2.28 min; purity = 100%. |
| | | | Absolute stereochemistry was assigned |
| | | | arbitrarily. |
| 28 | 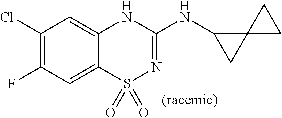 [SMILES: O=S1(=O)N=C(NC2CC23CC3)Nc2cc(Cl)c(F)cc21] | 6-chloro-7-fluoro-3- (spiro[2.2]pentan-1-ylamino)- 4H-benzo[e][1,2,4]thiadiazine 1,1-dioxide | LC/MS (ESI+): m/z 316.1 [M + H]⁺; Rt = 5.62 min. ¹H NMR (400 MHz, DMSO-d₆): δ 10.53 (s, 1H), 7.83 (br s, 1H), 7.77 (d, J = 8.0 Hz, 1H), 7.52 (br s, 1H), 3.00- 2.96 (m, 1H), 1.33-1.30 (m, 1H), 1.02-0.94 (m, 3H), 0.88-0.83 (m, 1H), 0.79-0.75 (m, 1H) |
| 29 | 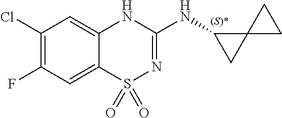 [SMILES: O=S1(=O)N=C(N[C@H]2CC23CC3)Nc2cc(Cl)c(F)cc21] | (S*)-6-chloro-7-fluoro-3- (spiro[2.2]pentan-1-ylamino)- 4H-benzo[e][1,2,4]-thiadiazine 1,1-dioxide | LC/MS (ESI+): m/z 316.2 [M + H]⁺; Rt = 5.69 min. ¹H NMR (400 MHz, DMSO-d₆): δ 10.53 (s, 1H), 7.82 (br s, 1H), 7.76 (d, J = 8.0 Hz, 1H), 7.52 (br s, 1H), 3.00- 2.97 (m, 1H), 1.33-1.30 (m, 1H), 1.02-0.94 (m. 3H), 0.88-0.83 (m, 1H), 0.80-0.76 (m, 1H). |
| | | | SFC: (Separation: Condition 3, |
| | | | Gradient 1; Analytical: Condition 1, |
| | | | Gradient 2) Rt = 1.64 min; purity = 98.9%. |
| | | | Absolute stereochemistry was assigned |
| | | | arbitrarily. |
| 30 | 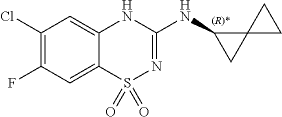 [SMILES: O=S1(=O)N=C(N[C@@H]2CC23CC3)Nc2cc(Cl)c(F)cc21] | (R*)-6-chloro-7-fluoro-3- (spiro[2.2]pentan-1-ylamino)- 4H-benzo[e][1,2,4]-thiadiazine 1,1-dioxide | LC/MS (ESI+): m/z 316.2 [M + H]⁺; Rt = 5.72 min. ¹H NMR (400 MHz, DMSO-d₆): δ 10.52 (s, 1H), 7.82 (br s, 1H), 7.76 (d, J = 8.0 Hz, 1H), 7.52 (br s, 1H), 3.00- 2.97 (m, 1H), 1.33-1.30 (m, 1H), 1.02-0.94 (m, 3H), 0.88-0.83 (m, 1H), 0.80-0.76 (m, 1H). |
| | | | SFC: (Separation: Condition 3, |
| | | | Gradient 1; Analytical: Condition 1, |
| | | | Gradient 2) Rt = 2.52 min; purity = 99.9%. |
| | | | Absolute stereochemistry was assigned |
| | | | arbitrarily. |
| 31 | 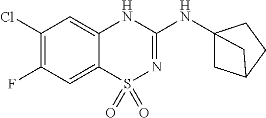 [SMILES: O=S1(=O)N=C(NC23CCC(C2)C3)Nc2cc(Cl)c(F)cc21] | 3-(bicyclo[2.1.1]hexan-1- ylamino)- 6-chloro-7-fluoro-4H- benzo[e][1,2,4]thiadiazine 1,1-dioxide | LC/MS (ESI+): m/z 330.1 [M + H]⁺; Rt = 8.12 min. ¹H NMR (400 MHz, DMSO-d₆): δ 10.36 (s, 1H), 7.85 (s, 1H), 7.76 (d. J = 7.9 Hz, 1H), 7.44 (d, J = 6.0 Hz, 1H), 2.35-2.32 (m, 1H), 1.88-1.84 (m, 2H), 1.83-1.77 (m, 2H), 1.67-1.63 (m, 2H), 1.47-1.42 (m, 2H). |

### Example 13. Synthesis of Compound 32 From Intermediate A7

A suspension of Intermediate 7 (80.0 mg, 0.32 mmol, 1 eq.), bicyclo[(0.1.1]pentan-1-amine hydrochloride (60.6 mg, 0.51 mmol, 1.6 eq.) and DIEA (143 μL, 0.82 mmol, 2.6 eq.) in dioxane (2.7 mL) was heated in a sealed vessel at 100° C. for 2 h. The reaction mixture was concentrated under reduced pressure and then partitioned between EtOAc (50 mL) and a water/brine mixture (2:1, 25 mL). The layers were separated and the organic layer was washed with a water/brine mixture (2:1, 2×25 mL), dried over, Na₂SO₄, filtered and concentrated under reduced pressure. The crude residue was purified by preparative HPLC (Condition 2, Gradient 1) to afford Compound 32 (55.0 mg) as a pale yellow solid. LC/MS (ESI+): m/z 300.3 [M+H]⁺; Rt=5.27 min. ¹H NMR (400 MHz, DMSO-d₆): δ 10.56 (br s, 1H), 7.98 (s, 1H), 7.81 (t, J=8.8 Hz, 1H), 7.28 (dd, J=11.6, 6.5 Hz, 1H), 2.47 (s, 1H), 2.08 (s, 6H).

The compounds reported in the following Table 5 were prepared via an analogous procedure.

TABLE 5

| Compound | | | |
|---|---|---|---|
| No. | Structure | Name | Analytical Data |
| 33 | 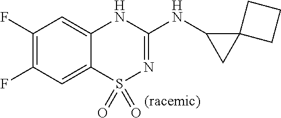 [SMILES: O=S1(=O)N=C(NC2CC23CCC3)Nc2cc(F)c(F)cc21] | 6,7-difluoro-3- (spiro[2.3]hexan-1- ylamino)-4H- benzo[e][1,2,4]thiadiazine 1,1-dioxide | LC/MS (ESI+): m/z 314.3 [M + H]⁺; Rt = 5.52 min. ¹H NMR (400 MHz, DMSO-d₆): δ 10.66 (s, 1H), 7.82 (dd, J = 9.3, 8.2 Hz, 1H), 7.52 (br s, 1H), 7.34 (br s, 1H), 2.54-2.52 (m, 1H), 2.15-2.06 (m, 3H), 2.00-1.93 (m. 3H), 0.93-0.90 (m, 1H), 0.56-0.54 (m, 1H). |
| 34 | 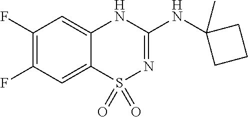 [SMILES: CC1(NC2=NS(=O)(=O)c3cc(F)c(F)cc3N2)CCC1] | 6,7-difluoro-3-((1- methylcyclobutyl)amino)- 4H- benzo[e][1,2,4]thiadiazine 1,1-dioxide | LC/MS (ESI+): m/z 302.2 [M + H]⁺; Rt = 5.35 min. ¹H NMR (400 MHz, DMSO-d₆): δ 10.46 (s, 1H), 7.79 (t, J = 8.8 Hz, 1H), 7.44 (s, 1H), 7.25 (dd. J = 11.5. 6.6 Hz, 1H). 2.30-2.23 (m, 2H), 2.04-1.98 (m, 2H), 1.85-1.77 (m, 2H), 1.48 (s, 3H). |

### Example 14. Synthesis of Compounds 35 and 36 from Intermediates A9 and A9′

A suspension of a mixture of Intermediates 9 and 9′ (200 mg, 0.74 mmol, 1 eq.), bicyclo[1.1.1]pentan-1-amine hydrochloride (142 mg, 1.19 mmol, 1.6 eq.) and DIEA (0.337 mL, 1.93 mmol, 2.6 eq.) in dioxane (6.3 mL) was stirred and heated at 100° C. for 2 h. The reaction mixture was concentrated under reduced pressure and then partitioned between EtOAc (150 mL) and a water/brine mixture (2:1, 50 mL). The layers were separated and the organic layer was washed with a water/brine mixture (2:1, 2×50 mL), dried over Na₂SO₄, filtered and concentrated under reduced pressure. The crude residue was purified by chiral SFC (Condition 1, Gradient 1) to afford Compound 35 (121 mg) and Compound 36 (30 mg) as pale-brown solids. Chiral SFC analyses data for separated regioisomers (column: Chiralpak OD-3 (4.6×100 mm), Mobile Phase (Isocratic Conditions): CO₂/EtOH, 75:25). Compound 35: Rt=1.12 min, Chiral SFC purity: 100%. Compound 36: Rt=2.11 min, Chiral SFC purity: 100%. Analytical data for Compound 35: LC/MS (ESI+): m/z 316.1 [M+H]⁺; Rt=5.72 min. ¹H NMR (400 MHz, DMSO-d₆): δ 10.58 (s, 1H), 8.04 (s, 1H), 7.90 (d, J=7.6 Hz, 1H), 7.27 (d, J=10.3 Hz, 1H), 2.48 (s, 1H), 2.08 (s, 6H). ¹H {¹⁹F}NMR (400 MHz, DMSO-d₆): δ 10.58 (s, 1H), 8.04 (s, 1H), 7.90 (s, 1H), 7.27 (s, 1H), 2.48 (s, 1H), 2.08 (s, 6H). Analytical data for Compound 36: LC/MS (ESI+): m/z 316.1 [M+H]⁺; Rt=5.56 min. ¹H NMR (400 MHz, DMSO-d₆): δ 10.78 (s, 1H), 7.87 (br s, 1H), 7.71 (t, J=8.3 Hz, 1H), 7.05 (d, J=9.0 Hz, 1H), 2.47 (s, 1H), 2.08 (s, 6H). ¹H {¹⁹F}NMR (400 MHz, DMSO-d₆): δ 10.78 (s, 1H), 7.87 (br s, 1H), 7.71 (d, J=9.0 Hz, 1H), 7.05 (d, J=9.0 Hz, 1H), 2.47 (s, 1H), 2.08 (s, 6H).

### Example 15. Synthesis of Compound 37 from Intermediate A11

A suspension of Intermediate A11 (550 mg, 1.86 mmol, 1 eq.), bicyclo[1.1.1]pentan-1-amine hydrochloride (334 mg, 2.79 mmol, 1.5 eq.) and DIEA (0.810 mL, 4.65 mmol, 2.5 eq.) in dioxane (15 mL) was stirred and heated at 100° C. for 3 h. The reaction mixture was concentrated under reduced pressure and then partitioned between EtOAc (150 mL) and a water/brine mixture (2:1, 50 mL). The layers were separated and the organic layer was washed with a water/brine mixture (2:1, 2×50 mL), dried over Na₂SO₄, filtered and concentrated under reduced pressure. The crude solid was triturated in EtOAc (2×7 mL), filtered and dried under reduced pressure to afford Compound 37 as an off-white solid (545 mg). LC/MS (ESI+): m/z 342.2 [M+H]⁺; Rt=5.41 min. ¹H NMR (400 MHz, DMSO-d₆): δ 10.38 (s, 1H), 7.98 (s, 1H), 7.61 (d, J=8.8 Hz, 1H), 7.42-7.44 (m, 2H), 2.48 (s, 1H), 2.09 (s, 6H).

### Example 16. Synthesis of Compound 38 from Intermediate A11′

Compound 38 was prepared according to the procedure as described for Compound 37 starting from Intermediate A11′ (15.0 mg, 0.051 mmol, 1 eq.) to afford Compound 38 as an off-white solid (10.0 mg). LC/MS (ESI+): m/z 342.2 [M+H]˜; Rt=5.06 min. ¹H NMR (400 MHz, DMSO-d₆): δ 10.48 (s, 1H), 7.63 (s, 1H), 7.45 (dd, J=7.9, 1.2 Hz, 1H), 7.40 (t, J=7.9 Hz, 1H), 7.15 (dd, J=8.0, 1.3 Hz, 1H), 2.48 (s, 1H), 2.09 (s, 6H).

### Example 17. Synthesis of Compound 39 from Intermediate A13

Compound 39 was prepared according to the procedure described for Compound 37 starting from Intermediate A13 (50.0 mg, 0.15 mmol, 1 eq.) to afford Compound 39 as a white solid (34.0 mg). LC/MS (ESI+): m/z 390.1 [M+H]⁺; Rt=5.51 min. ¹H NMR (400 MHz, DMSO-d₆): δ 10.35 (s, 1H), 7.95 (s, 1H), 7.61-7.58 (m, 2H), 7.42 (d, J=8.6 Hz, 1H), 2.47 (s, 1H), 2.08 (s, 6H).

### Example 18. Synthesis of Compound 40 from Compound 37

To an argon-purged suspension of Compound 37 (70.0 mg, 0.21 mmol, 1 eq.), trimethylboroxine (57.7 μL, 0.41 mmol, 2 eq.) and Cs₂CO₃ (133 mg, 0.41 mmol, 2 eq.) in anhydrous dioxane (4 mL) was added Pd(dppf)Cl₂-DCM (16.7 mg, 0.021 mmol, 0.1 eq.). The reaction mixture was degassed by bubbling Argon for 5 minutes and stirred at 100° C. for 18 h. The reaction mixture was cooled to room temperature and partitioned between EtOAc (30 mL) and water (10 mL). The layers were separated and the aqueous layer was extracted with EtOAc (3×30 mL). The combined organic layers were washed with brine (20 mL), dried over Na₂SO₄, filtered and concentrated under reduced pressure. The crude residue was purified by silica gel flash chromatography (Condition 1, Gradient 2) followed by preparative HPLC (Condition 1, Gradient 1) to afford Compound 40 (28.0 mg) as a white solid. LC/MS (ESI+): m/z 278.3 [M+H]⁺; Rt=5.00 min. ¹H NMR (400 MHz, DMSO-d₆): δ 10.21 (s, 1H), 7.65 (s, 1H), 7.54 (d, J=8.0 Hz, 1H), 7.07 (dd, J=8.0, 1.7 Hz, 1H), 6.95 (d, J=1.7 Hz, 1H), 2.47 (s, 1H), 2.34 (s, 3H), 2.08 (s, 6H).

### Example 19. Synthesis of Compound 41 from Compound 37

To an argon-purged solution of Compound 37 and zinc cyanide (16.5 mg, 0.14 mmol, 1.6 eq.) in anhydrous DMF (0.7 mL) was added Pd(PPh₃)₄ (12.2 mg, 0.011 mmol, 0.12 eq.). The reaction mixture was degassed by bubbling Argon for 5 minutes and stirred at 100° C. for 18 h. The reaction mixture was cooled to room temperature and partitioned between EtOAc (15 mL) and water (10 mL). The layers were separated and the aqueous layer was extracted with EtOAc (2×20 mL). The combined organic layers were washed with brine (3×20 mL), dried over Na₂SO₄, filtered and concentrated under reduced pressure. The crude residue was purified by silica gel flash chromatography (Condition 1, Gradient 2) to afford Compound 41 (10.0 mg) as a white solid. LC/MS (ESI+): m/z 289.3 [M+H]⁺; Rt=5.05 min. ¹H NMR (400 MHz, DMSO-d₆): δ 10.58 (s, 1H), 8.08 (s, 1H), 7.85 (d, J=8.4 Hz, 1H), 7.68-7.65 (m, 2H), 2.48 (s, 1H), 2.09 (s, 6H).

| Compound | LC/MS Method Number | Rt (min) | [M + H]⁺ |
|---|---|---|---|
| 1 | 1 | 6.99 | 282.1 |
| 2 | 2 | 5.35 | 284.2 |
| 3 | 2 | 5.25 | 300.1 |
| 4 | 2 | 5.33 | 296.2 |
| 5 | 2 | 5.09 | 284.2 |
| 6 | 2 | 5.05 | 284.4 |
| 7 | 2 | 5.06 | 284.4 |
| 8 | 2 | 4.94 | 282.2 |
| 9 | 2 | 4.93 | 282.4 |
| 10 | 2 | 4.93 | 282.4 |
| 11 | 1 | 7.51 | 296.4 |
| 12 | 2 | 4.99 | 284.5 |
| 13 | 2 | 5.10 | 296.4 |
| 14 | 2 | 5.74 | 316.2 |
| 15 | 1 | 8.34 | 330.1 |
| 16 | 2 | 5.93 | 330.1 |
| 17 | 2 | 5.92 | 330.1 |
| 18 | 1 | 8.29 | 330.1 |
| 19 | 1 | 7.79 | 334.1 |
| 20 | 2 | 5.75 | 318.1 |
| 21 | 2 | 5.84 | 318.2 |
| 22 | 2 | 5.83 | 318.2 |
| 23 | 1 | 7.83 | 358.1 |
| 24 | 1 | 7.95 | 318.1 |
| 25 | 2 | 5.53 | 304.1 |
| 26 | 2 | 5.56 | 304.2 |
| 27 | 2 | 5.58 | 304.2 |
| 28 | 2 | 5.62 | 316.1 |
| 29 | 2 | 5.69 | 316.2 |
| 30 | 2 | 5.72 | 316.2 |
| 31 | 1 | 8.12 | 330.1 |
| 32 | 2 | 5.27 | 300.3 |
| 33 | 2 | 5.52 | 314.3 |
| 34 | 2 | 5.35 | 302.2 |
| 35 | 2 | 5.72 | 316.1 |
| 36 | 2 | 5.56 | 316.1 |
| 37 | 2 | 5.41 | 342.2 |
| 38 | 2 | 5.06 | 342.2 |
| 39 | 2 | 5.51 | 390.1 |
| 40 | 2 | 5.00 | 278.3 |
| 41 | 2 | 5.05 | 289.3 |

| Compound | ¹H NMR data |
|---|---|
| 1 | ¹H NMR (400 MHz, DMSO-d₆): δ 10.44 (s, 1H), 7.80 (s, 1H), 7.50 (dd, J = 7.6, 2.9 Hz, 1H), |
| | 7.45 (dt, J = 8.8, 2.9 Hz, 1H), 7.25 (dd, J = 8.8, 4.4 Hz, 1H), 2.47 (s, 1H), 2.08 (s, 6H). |
| 2 | ¹H NMR (400 MHz, DMSO-d₆): δ 10.34 (s, 1H), 7.48 (dd, J = 7.6, 3.0 Hz, 1H), 7.44 (dt, J = |
| | 8.7, 2.8 Hz, 1H), 7.23 (s, 1H), 7.21 (dd, J = 8.9, 4.3 Hz, 1H), 2.31-2.23 (m, 2H), 2.04-1.98 |
| | (m, 2H), 1.85-1.77 (m, 2H), 1.49 (s, 3H). |
| 3 | ¹H NMR (400 MHz, DMSO-d₆): δ 10.71 (br s, 1H), 8.02 (s, 1H), 7.53 (dd, J = 7.6, 2.9 Hz, |
| | 1H), 7.46 (dt, J = 8.7, 2.9 Hz, 1H), 7.28 (dd, J = 9.0, 4.4 Hz, 1H), 2.42 (d, J = 2.2 Hz, 6H). |
| 4 | ¹H NMR (400 MHz, DMSO-d₆): δ 10.37 (s, 1H), 7.61 (s, 1H), 7.50 (dd, J = 7.6, 2.9 Hz, 1H), |
| | 7.45 (dt, J = 8.8, 2.9 Hz, 1H), 7.23 (dd, J = 9.0, 4.3 Hz, 1H), 2.35-2.32 (m, 1H), 1.89-1.85 |
| | (m, 2H), 1.84-1.78 (m, 2H), 1.67-1.63 (m, 2H), 1.47-1.41 (m, 2H). |
| 5 | ¹H NMR (400 MHz, DMSO-d₆): δ 10.51 (s, 1H), 7.72-7.35 (m, 3H), 7.31 (s, 1H), 2.47-2.42 |
| | (m, 1H), 1.09 (s, 3H), 1.02 (s, 3H), 0.76 (dd, J = 7.8. 5.2 Hz, 1H), 0.43 (t, J = 4.6 Hz, 1H). |
| 6 | ¹H NMR (400 MHz, DMSO-d₆): δ 10.51 (br s, 1H), 7.74-7.37 (m, 3H), 7.31 (br s, 1H), |
| | 2.47-2.42 (m, 1H), 1.09 (s, 3H), 1.02 (s, 3H), 0.76 (dd, J = 7.7, 5.2 Hz, 1H), 0.43 (t, J = 4.7 |
| | Hz, 1H). |
| 7 | ¹H NMR (400 MHz, DMSO-d₆): δ 10.55 (br s, 1H), 7.74-7.37 (m, 3H), 7.31 (br s, 1H), |
| | 2.47-2.42 (m, 1H), 1.09 (s, 3H), 1.02 (s, 3H), 0.76 (dd, J = 7.7, 5.2 Hz, 1H), 0.43 (t, J = 4.7 |
| | Hz, 1H). |
| 8 | ¹H NMR (400 MHz, DMSO-d₆): δ 10.51 (s, 1H), 7.57 (br s, 1H), 7.51-7.42 (m, 2H), 7.32 (s, |
| | 1H), 3.00-2.96 (m, 1H), 1.32-1.29 (m, 1H), 1.02-0.93 (m, 3H), 0.87-0.83 (m, 1H), 0.79- |
| | 0.74 (m, 1H). |
| 9 | ¹H NMR (400 MHz, DMSO-d₆): δ 10.32 (br s, 1H), 7.62 (br s, 1H), 7.52-7.40 (m, 2H), 7.31 |
| | (br s, 1H), 3.00-2.95 (m, 1H), 1.32-1.29 (m, 1H), 1.03-0.93 (m, 3H), 0.87-0.83 (m, 1H), |
| | 0.80-0.74 (m, 1H). |
| 10 | ¹H NMR (400 MHz, DMSO-d₆): δ 10.20 (br s, 1H), 7.67 (br s, 1H), 7.52-7.40 (m, 2H), 7.31 |
| | (br s, 1H), 3.00-2.95 (m, 1H), 1.32-1.29 (m, 1H), 1.03-0.93 (m, 3H), 0.87-0.83 (m, 1H), |
| | 0.80-0.74 (m, 1H). |
| 11 | ¹H NMR (400 MHz, DMSO-d₆): δ 10.57 (s, 1H), 7.51 (dd, J = 7.6, 2.9 Hz, 1H), 7.47 (dt, J = |
| | 8.7, 2.9 Hz, 1H), 7.34 (br s, 2H), 2.56-2.52 (m, 1H), 2.18-2.06 (m, 3H), 2.03-1.91 (m, 3H), |
| | 0.93-0.90 (m, 1H), 0.56-0.54 (m, 1H). |
| 12 | ¹H NMR (400 MHz, DMSO-d₆): δ 10.26 (br s, 1H), 7.62 (br s, 1H), 7.49-7.42 (m, 2H), 7.35 |
| | (br s, 1H), 1.61 (q, J = 7.4 Hz, 2H), 0.91 (t, J = 7.4 Hz, 3H), 0.76-0.68 (m, 4H). |
| 13 | ¹H NMR (400 MHz, DMSO-d₆): δ 10.32 (s, 1H), 7.72 (br s, 1H), 7.50-7.43 (m, 2H), 7.37 (br |
| | s, 1H), 1.46-1.39 (m, 1H), 0.72-0.69 (m, 2H), 0.66-0.63 (m, 2H), 0.42-0.37 (m, 2H), 0.22- |
| | 0.18 (m, 2H). |
| 14 | ¹H NMR (400 MHz, DMSO-d₆): δ 10.44 (s, 1H), 8.05 (s, 1H), 7.77 (d, J = 7.9 Hz, 1H), 7.46 |
| | (d, J = 6.1 Hz, 1H), 2.48 (s, 1H), 2.09 (s, 6H). |
| 15 | ¹H NMR (400 MHz, DMSO-d₆): δ 10.57 (s, 1H), 7.78 (d, J = 7.9 Hz, 1H), 7.53 (br s, 2H), |
| | 2.58-2.54 (m, 1H), 2.17-2.05 (m, 3H), 2.01-1.94 (m, 3H), 0.94-0.91 (m, 1H), 0.58-0.54 (m, |
| | 1H). |
| 16 | ¹H NMR (400 MHz, DMSO-d₆): δ 10.57 (s, 1H), 7.78 (d, J = 7.9 Hz, 1H), 7.53 (br s, 2H), |
| | 2.58-2.54 (m, 1H), 2.17-2.05 (m, 3H), 2.01-1.94 (m, 3H), 0.95-0.89 (m, 1H), 0.59-0.53 (m, |
| | 1H). |
| 17 | ¹H NMR (400 MHz, DMSO-d₆): δ 10.57 (s, 1H), 7.78 (d, J = 7.9 Hz, 1H), 7.53 (br s, 2H), |
| | 2.58-2.54 (m, 1H), 2.17-2.05 (m, 3H), 2.01-1.94 (m, 3H), 0.95-0.89 (m, 1H), 0.59-0.53 (m, |
| | 1H). |
| 18 | ¹H NMR (400 MHz, DMSO-d₆): δ 10.40 (s, 1H), 7.99 (s, 1H), 7.76 (d, J = 7.9 Hz, 1H), 7.46 |
| | (d, J = 6.1 Hz, 1H), 1.95 (s, 6H), 1.23 (s, 3H). |
| 19 | ¹H NMR (400 MHz, DMSO-d₆): δ 10.69 (s, 1H), 8.25 (s, 1H), 7.80 (d, J = 7.9 Hz, 1H), 7.48 |
| | (d, J = 6.1 Hz, 1H), 2.42 (d, J = 2.2 Hz, 6H). |
| 20 | ¹H NMR (400 MHz, DMSO-d₆): δ 10.53 (s, 1H), 7.77 (d, J = 8.0 Hz, 1H), 7.52 (br s, 2H), |
| | 2.47-2.42 (m, 1H), 1.09 (s, 3H), 1.01 (s, 3H), 0.78-0.75 (m, 1H), 0.46-0.43 (m, 1H). |
| 21 | ¹H NMR (400 MHz, DMSO-d₆): δ 10.53 (s, 1H), 7.76 (d, J = 8.0 Hz, 1H), 7.52 (br s, 2H), |
| | 2.47-2.42 (m, 1H), 1.09 (s, 3H), 1.02 (s, 3H), 0.78-0.75 (m, 1H), 0.46-0.43 (m, 1H). |
| 22 | ¹H NMR (400 MHz, DMSO-d₆): δ 10.55 (s, 1H), 7.76 (d, J = 8.0 Hz, 1H), 7.51 (br s, 2H), |
| | 2.47-2.42 (m, 1H), 1.09 (s, 3H), 1.02 (s, 3H), 0.78-0.75 (m, 1H), 0.46-0.43 (m, 1H). |
| 23 | ¹H NMR (400 MHz, DMSO-d₆): δ 10.65 (br s, 1H), 8.41 (br s, 1H), 7.81 (d, J = 8.0 Hz, 1H), |
| | 7.62 (br s, 1H), 1.42-1.38 (m, 2H), 1.28-1.24 (m, 2H). |
| 24 | ¹H NMR (400 MHz, DMSO-d₆): δ 10.33 (s, 1H), 7.75 (d, J = 8.0 Hz, 1H), 7.48 (s, 1H), 7.42 |
| | (d, J = 6.0 Hz, 1H), 2.31-2.23 (m, 2H), 2.05-1.99 (m, 2H), 1.86-1.78 (m, 2H), 1.49 (s, 3H). |
| 25 | ¹H NMR (400 MHz, DMSO-d₆): δ 10.48 (br s, 1H), 7.84 (br s, 1H), 7.76 (d, J = 8.0 Hz, 1H), |
| | 7.57 (br s, 1H), 2.40-2.36 (m, 1H), 1.07 (d, J = 6.1 Hz, 3H), 0.99-0.91 (m, 1H), 0.73-0.69 |
| | (m, 1H), 0.61-0.56 (m, 1H). |
| 26 | ¹H NMR (400 MHz, DMSO-d₆): δ 10.49 (br s, 1H), 7.82 (br s, 1H), 7.75 (d, J = 7.9 Hz. 1H), |
| | 7.55 (br s, 1H), 2.40-2.36 (m, 1H), 1.07 (d, J = 6.1 Hz, 3H), 0.99-0.91 (m, 1H), 0.73-0.69 |
| | (m, 1H), 0.60-0.56 (m, 1H). |
| 27 | ¹H NMR (400 MHz, DMSO-d₆): δ 10.50 (br s, 1H), 7.82 (br s, 1H), 7.75 (d, J = 7.9 Hz, 1H), |
| | 7.56 (br s, 1H), 2.40-2.36 (m, 1H), 1.07 (d, J = 6.0 Hz, 3H), 0.99-0.91 (m, 1H), 0.73-0.69 |
| | (m, 1H), 0.60-0.56 (m, 1H). |
| 28 | ¹H NMR (400 MHz, DMSO-d₆): δ 10.53 (s, 1H), 7.83 (br s, 1H), 7.77 (d, J = 8.0 Hz, 1H), |
| | 7.52 (br s, 1H), 3.00-2.96 (m, 1H), 1.33-1.30 (m, 1H), 1.02-0.94 (m, 3H), 0.88-0.83 (m, |
| | 1H), 0.79-0.75 (m, 1H). |
| 29 | ¹H NMR (400 MHz, DMSO-d₆): δ 10.53 (s, 1H), 7.82 (br s, 1H), 7.76 (d, J = 8.0 Hz, 1H), |
| | 7.52 (br s, 1H), 3.00-2.97 (m, 1H), 1.33-1.30 (m, 1H), 1.02-0.94 (m, 3H), 0.88-0.83 (m, |
| | 1H), 0.80-0.76 (m, 1H). |
| 30 | ¹H NMR (400 MHz, DMSO-d₆): δ 10.52 (s, 1H), 7.82 (br s, 1H), 7.76 (d, J = 8.0 Hz, 1H), |
| | 7.52 (br s, 1H), 3.00-2.97 (m, 1H), 1.33-1.30 (m, 1H), 1.02-0.94 (m, 3H), 0.88-0.83 (m, |
| | 1H), 0.80-0.76 (m, 1H). |
| 31 | ¹H NMR (400 MHz, DMSO-d₆): δ 10.36 (s, 1H), 7.85 (s, 1H), 7.76 (d, J = 7.9 Hz, 1H), 7.44 |
| | (d, J = 6.0 Hz, 1H), 2.35-2.32 (m, 1H), 1.88-1.84 (m, 2H), 1.83-1.77 (m, 2H), 1.67-1.63 |
| | (m, 2H), 1.47-1.42 (m, 2H). |
| 32 | ¹H NMR (400 MHz, DMSO-d₆): δ 10.56 (br s, 1H), 7.98 (s, 1H), 7.81 (t, J = 8.8 Hz, 1H), |
| | 7.28 (dd, J = 11.6, 6.5 Hz, 1H), 2.47 (s, 1H), 2.08 (s, 6H). |
| 33 | ¹H NMR (400 MHz, DMSO-d₆): δ 10.66 (s, 1H), 7.82 (dd, J = 9.3, 8.2 Hz, 1H), 7.52 (br s, |
| | 1H), 7.34 (br s, 1H), 2.54-2.52 (m, 1H), 2.15-2.06 (m, 3H), 2.00-1.93 (m, 3H), 0.93-0.90 |
| | (m, 1H), 0.56-0.54 (m, 1H). |
| 34 | ¹H NMR (400 MHz, DMSO-d₆): δ 10.46 (s, 1H), 7.79 (t, J = 8.8 Hz, 1H), 7.44 (s, 1H), 7.25 |
| | (dd, J = 11.5, 6.6 Hz, 1H), 2.30-2.23 (m, 2H), 2.04-1.98 (m, 2H), 1.85-1.77 (m, 2H), 1.48 |
| | (s, 3H). |
| 35 | ¹H NMR (400 MHz, DMSO-d₆): δ 10.58 (s, 1H), 8.04 (s, 1H), 7.90 (d, J = 7.6 Hz, 1H), 7.27 |
| | (d, J = 10.3 Hz, 1H), 2.48 (s, 1H), 2.08 (s, 6H). |
| | ¹H {¹⁹F} NMR (400 MHz, DMSO-d₆): δ 10.58 (s, 1H), 8.04 (s, 1H), 7.90 (s, 1H), 7.27 (s, |
| | 1H), 2.48 (s, 1H), 2.08 (s, 6H). |
| 36 | ¹H NMR (400 MHz, DMSO-d₆): δ 10.78 (s, 1H), 7.87 (br s, 1H), 7.71 (t, J = 8.3 Hz, 1H), |
| | 7.05 (d, J = 9.0 Hz, 1H), 2.47 (s, 1H), 2.08 (s, 6H). |
| | ¹H {¹⁹F} NMR (400 MHz, DMSO-d₆): δ 10.78 (s, 1H), 7.87 (br s, 1H), 7.71 (d, J = 9.0 Hz, |
| | 1H), 7.05 (d, J = 9.0 Hz, 1H), 2.47 (s, 1H), 2.08 (s, 6H). |
| 37 | ¹H NMR (400 MHz, DMSO-d₆): δ 10.38 (s, 1H), 7.98 (s, 1H), 7.61 (d, J = 8.8 Hz, 1H), |
| | 7.42-7.44 (m, 2H), 2.48 (s, 1H), 2.09 (s, 6H). |
| 38 | ¹H NMR (400 MHz, DMSO-d₆): δ 10.48 (s, 1H), 7.63 (s, 1H), 7.45 (dd, J = 7.9, 1.2 Hz, 1H), |
| | 7.40 (t, J = 7.9 Hz, 1H), 7.15 (dd, J = 8.0, 1.3 Hz, 1H), 2.48 (s, 1H), 2.09 (s, 6H). |
| 39 | ¹H NMR (400 MHz, DMSO-d₆): δ 10.35 (s, 1H), 7.95 (s, 1H), 7.61-7.58 (m, 2H), 7.42 (d, J = |
| | 8.6 Hz, 1H), 2.47 (s, 1H), 2.08 (s, 6H). |
| 40 | ¹H NMR (400 MHz, DMSO-d₆): δ 10.21 (s, 1H), 7.65 (s, 1H), 7.54 (d, J = 8.0 Hz, 1H), 7.07 |
| | (dd, J = 8.0, 1.7 Hz, 1H), 6.95 (d, J = 1.7 Hz, 1H), 2.47 (s, 1H), 2.34 (s, 3H), 2.08 (s, 6H). |
| 41 | ¹H NMR (400 MHz, DMSO-d₆): δ 10.58 (s, 1H), 8.08 (s, 1H), 7.85 (d, J = 8.4 Hz, 1H), |
| | 7.68-7.65 (m, 2H), 2.48 (s, 1H), 2.09 (s, 6H). |

| | | Chiral SFC | Chiral SFC | | |
|---|---|---|---|---|---|
| | | Separation | Analysis | | Chiral |
| | | Conditions | Conditions | | SFC |
| | Assigned | (Mobile Phase, | (Mobile Phase, | Rt | purity |
| Compound | Stereochemistry¹ | Isocratic) | Isocratic) | (min) | (%) |
| 6 | S* | CO₂/MeOH, 75:25 | CO₂/MeOH, 80:20 | 1.16 | 100 |
| 7 | R* | | | 2.47 | 100 |
| 9 | S* | CO₂/(EtOH + 0.3% | CO₂/(EtOH + 0.3% | 1.74 | 100 |
| 10 | R* | v/v i-PrNH₂), 70:30 | v/v i-PrNH₂), 70:30 | 2.93 | 99.5 |
| 16 | S* | CO₂/EtOH, 60:40 | CO₂/EtOH, 70:30 | 1.48 | 97.4 |
| 17 | R* | | | 4.38 | 100 |
| 21 | S* | CO₂/MeOH, 75:25 | CO₂/MeOH, 75:25 | 1.12 | 100 |
| 22 | R* | | | 2.57 | 100 |
| 26 | S*, S* | CO₂/MeOH, 75:25 | CO₂/MeOH, 75:25 | 1.61 | 100 |
| 27 | R*, R* | | | 2.28 | 100 |
| 29 | S* | CO₂/MeOH, 75:25 | CO₂/MeOH, 75:25 | 1.64 | 98.9 |
| 30 | R* | | | 2.52 | 99.9 |

¹Stereochemistry assigned arbitrarily.

### Example 20. Glucose Stimulated Insulin Secretion Assay (GSIS)

INS-1E, rat pancreatic tumor (insulinoma) cells were used in the GSIS assay. INS-1E cells were thawed and incubated in culture medium (CM) (RPMI 1640+Glucose q.s., HEPES 25 mM, Na-Pyruvat 1 mM, 2-Mercaptoethanol 50 μM, Fetal Calf Serum; heat inactivated (FCS) 10%, Penicillin/Streptomycin 100 U/ml) at 37° C.; 5% CO2 in a humidified atmosphere. 96 welled plates are coated with Matrigel by incubating the plates with 1 ml pre-diluted 1:10 with cold basal medium with 100 μl/well at 37° C. for 1 hour. Matrigel is then removed and replaced with 100 μl CM. Cells are then seeded into the wells as 70 000 cells/well. CM is refreshed with 200 μl fresh CM on the third day. CM is refreshed again with 200 μl fresh CM on the sixth day. GSIS assay is performed on the seventh day and cells in wells are washed with PKrebs®BSA and incubated with 100 μL/well for 2 h. Secretion plates are placed in an incubator at 37° C., 5% C02 and saturated humidity for 60 min. Meanwhile, lysis solution is prepared by adding 1 tablet of protease inhibitor/10 mL TETG solution and kept at 4° C. until the end of the experiment. The other incubation buffers are prepared without adding pharmacological compounds, aliquoted and then placed in a water bath at 37° C. The samples are prepared including negative (βKrebs®B0=0 mM Glucose) and positive controls (PKrebs®B20=20 mM Glucose) and each compound to be tested is diluted in DMSO as having a series of final concentration including 10.00, 4.00, 1.60, 0.64, 0.26, 0.10, 0.04, 0.02 μM.

After 2 hours of starvation, glucose stimulation and treatments are initiated. KRB Buffer from cells is removed by pipetting. The solutions are homogenized and the cells are treated with 90 L/well. 10 μl from 10-fold compound dilution is added to the assay plate (final 100 μl/well) according to the following layout for each compound to be tested.

Incubation time is 40 minutes. Plates are placed in an incubator at 37° C., 5% CO₂ and saturated humidity. After incubation, 80 μL of supernatants are collected without disrupting the cell layer and stored at 4° C. or on ice until centrifugation. Remaining medium are removed from each well and 100 μL/well of cold lysis solution is added and incubated for minimum 2 minutes at room temperature. Cell lysis is observed under microscope and waited for maximum for 5 more minutes until the lysis is complete. Cell lysates are collected and all samples including supernatants are centrifuged at 700×g, 5 min at 4° C. 30 μL of each samples are transferred into aliquots for ELISA. ELISA is performed with HTRF Assay Kit comprising an insulin high range kit supplied by Cisbio/Perkin Elmer. 5 μl of each Insulin standard and 5 μl of either supernatant or lysate are transferred on HTRF plate. 40 μl/well of Anti-insulin—XL665 and 20 μl/well of Anti-insulin—EU are added to each well and the plate is read on HTRF compatible reader (fluorescence 620 nm & 665 nm).

GSIS experiments were also performed in other cell lines, e.g., EndoC-βH1, a canonical cell line for screening new therapeutic compounds.

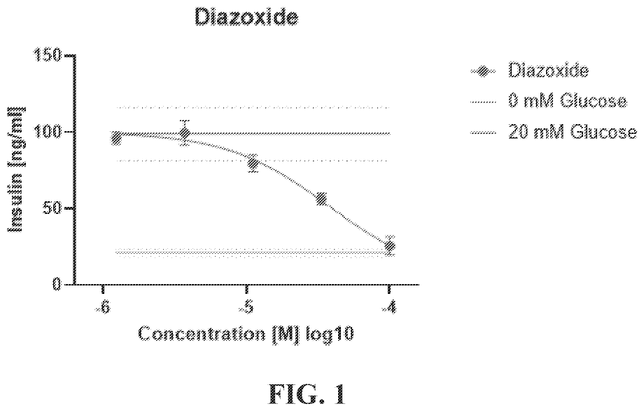

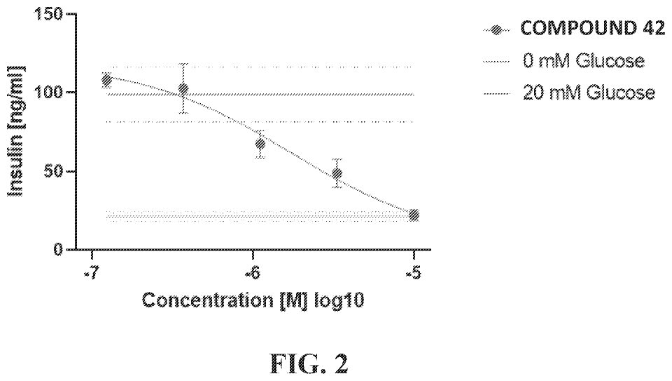

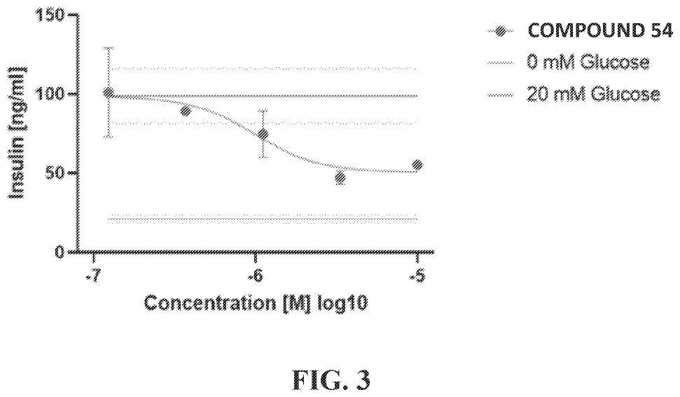

FIG. 1 illustrates the inhibition of insulin by diazoxide in the GSIS assay; FIG. 2 illustrates the inhibition of insulin by Compound 42 in the GSIS assay; FIG. 3 illustrates the inhibition of insulin by Compound 54 in the GSIS assay.

The ability to inhibit insulin of certain compounds of the present invention is given in Table B, below, where the data is provided according to the following key: A is equal to 50% inhibition or greater; B is equal to 25-50% inhibition; C is equal to 10-25% inhibition; and D is equal to 10% or less inhibition.

TABLE B
GSIS test results performed in INS-1E cells

| Compound No. | % inhibition of Insulin (10 μM)* |
|---|---|
| 51 | C |
| 46 | A |
| 58 | A |
| 52 | C |
| 47 | C |
| 60 | A |
| 49 | A |
| 43 | C |
| 72 | B |
| 71 | C |
| 73 | A |
| 63 | A |
| 64 | B |
| 62 | B |
| 1 | A |
| 14 | A |
| 65 | B |
| 69 | A |

*% of inhibition of insulin secretion from Rat pancreatic islets

In Table C, the IC50 values are provided as follows: A is less than 0.5 uM; B is 0.5 uM to 1 uM; C is 1 uM to 2 uM; and D is 2 uM or greater.

TABLE C
GSIS Assay Results in EndoC-βH1
Cell Line with Compound IC50

| Compound | IC50 | Compound | IC50 | Compound | IC50 |
|---|---|---|---|---|---|
| No. | (μM) | No. | (μM) | No. | (μM) |
| 1 | A | 25 | C | 21 | B |
| 14 | A | 28 | A | 22 | C |
| 15 | A | 17 | A | 26 | C |
| 19 | D | 2 | A | 27 | D |
| 24 | A | 3 | D | 29 | D |
| 20 | A | 4 | B | 30 | A |
| 16 | B | 5 | C | | |
| 16 | B | 8 | C | | |

In Table D, the data is provided according to the following key: A is equal to 5000 inhibition or greater; B is equal to 25-50% inhibition; C is equal to 10-25% inhibition; and D is equal to 1000 or less inhibition.

TABLE D
GSIS Assay Results in INS-1E Cell Line: Average Inhibition (%)

| Compound | % inhibition | Compound | % inhibition |
|---|---|---|---|
| No. | of Insulin | No. | of Insulin |
| 1 | A | 20 | A |
| 14 | A | 16 | A |
| 11 | B | 25 | C |
| 15 | A | 28 | A |
| 19 | C | 17 | A |
| 24 | A | 4 | A |
| 23 | D | 5 | A |
| 18 | C | 8 | A |
| 21 | A | 13 | D |
| 22 | A | 6 | A |
| 26 | A | 7 | B |
| 27 | A | 9 | B |
| 29 | A | 10 | A |
| 12 | C | | |

TABLE E
GSIS Assay Results in INS-1E Cell Line with Compound IC50

| Compound No. | IC50 (μM) |
|---|---|
| 1 | B |
| 14 | A |
| 11 | B |
| 15 | A |

These results show that the compounds according to the invention are able to reduce insulin secretion.

### Example 2. Identification of Selective β-Cell K_{ATP} Channel Activator Compounds

The example set forth below describes experiments to identify and characterize selective pancreatic β-cell K_{ATP} channel activator compounds employing a non-radiological rubidium efflux assay in concert with electrophysiological techniques. SUR1 and Kir6.2 (the pancreatic K_{ATP} channel isoform) will be co-expressed in COSm6 cells, and KATP channel activation will be interrogated using a Rb efflux assay following a Martin G. M. et al., Elife, 2017, 6 and Li, J. B. et al., J Biol Chem., 2013, 288(32): 23038-49 with modifications. Briefly, COSm6 cells (a common mammalian cell line that does not express endogenous K_{ATP} channels) will be transiently transfected with cDNAs encoding wild type SUR1 and Kir6.2 using Fugene6. Transfected cells will be cultured in medium containing 5 mM RbCl overnight. The next day, cells will be washed quickly twice in Ringer's solution with no RbCl and Rb efflux measured over a 30 min period with or without candidate compound (e.g., a compound described herein) at two different concentrations: 1 and 10 M. Diazoxide, a known K_{ATP} activator, at 1, 10 and 200 M will be tested in parallel and used as positive control. DMSO at 0.1% will be used as vehicle-treated negative control. Untransfected cells will be included in the assay to assess background efflux. At the end of the 30-min incubation, Ringer solution will be harvested, and cells will be lysed in Ringer's solution plus 1% Triton X-100. Rb concentration in both the efflux solution and cell lysate will be measured using an Atomic Adsorption Instrument Ion Channel Reader ICR 8100^{TM}(available from Aurora Biomed). For each experiment (i.e., each transfection), duplicates (i.e., two identically prepared wells of cells) will be performed as technical repeats and the average taken as the data point for a single experiment. Three separate transfections will be performed for each test compound as three biological repeats to mitigate potential variations arising from variable transfection efficiency. The three data points will be averaged, and standard error of the mean calculated as final data for each of the two concentrations for each compound. Compounds stimulating effects will be tested again by including 10 μM glibenclamide, a K_{ATP} channel specific inhibitor, to validate the increased efflux is due to increased K_{ATP} channel activity. Next, these test compounds will be tested on K_{ATP} channels formed by SUR2A and Kir6.2, and SUR2B and Kir6.2, to assess K_{ATP} isoform specificity.

The % efflux will be calculated by dividing Rb in the efflux solution by the total Rb in both the efflux solution and the cell lysate. The efflux from un-transfected COSm6 cells will be considered as background noise and subtracted from experimental values. As only two concentrations will be tested in the initial experiment, % activation will only be compared at each concentration (1, 10 μM) of each compound to cells treated with diazoxide at the same concentrations and at 200 μM, which will be considered maximum stimulation.

Dose response electrophysiology experiments will subsequently be conducted to measure the effective concentrations of the compounds, e.g., the compounds as described herein, in activating K_{ATP} channels, using techniques such as inside-out patch clamp. At least 5 concentrations will be tested and the concentrations to be tested for each compound will be determined based on the results from the Rb efflux assay as described above. For each compound and each concentration, three biological repeats each with two technical repeats will be performed. Diazoxide will be used as a positive control and glibenclamide will be used as a negative control. Where solvent such as DMSO is used to dissolve the compounds, DMSO at concentrations matching those used to dissolve the compounds will be used as vehicle controls. Electrophysiological data will be used to construct dose response curves for the leading compounds as described previously for other pharmacological modulators and EC50 calculated using GraphPad.

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from.

1. A compound having the structure of Formula (I): 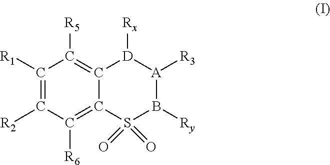
or a pharmaceutically acceptable salt, tautomer, stereoisomer, hydrate, or isotope thereof, wherein:
A is C(R′);
each of B and D are independently N; wherein A and B or A and D are connected via a double or single bond;
each of R₁, R₂, R₅, and R₆ are independently hydrogen, C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano; wherein A and B form a double bond and Rₓ is hydrogen or wherein A and D form a double bond and R_{y} is hydrogen;
R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), wherein alkyl, alkenyl, alkynyl, heteroalkyl, haloalkyl, cycloalkyl, and heterocyclyl are each optionally substituted with one or more R₄;
R′ is absent, hydrogen, or C1-C6 alkyl;
each R₄ is independently C1-C6 alkyl, C1-C6 heteroalkyl, C1-C6 haloalkyl, cycloalkyl, heterocyclyl, fluorine, chlorine, bromine, or iodine;
R^{A} is hydrogen, C1-C6 alkyl, C2-C6 alkenyl, C1-C6 alkynyl, C1-C6 heteroalkyl, C1-C6 haloalkyl, cycloalkyl, or heterocyclyl, wherein alkyl, alkenyl, alkynyl, heteroalkyl, haloalkyl, cycloalkyl, and heterocyclyl are each optionally substituted with one or more R₇;
each of R^{B} and R^{C} is independently hydrogen, C1-C6 alkyl, C2-C6 alkenyl, C1-C6 alkynyl, C1-C6 heteroalkyl, C1-C6 haloalkyl, cycloalkyl, heterocyclyl, wherein alkyl, alkenyl, alkynyl, heteroalkyl, haloalkyl, cycloalkyl, and heterocyclyl are each optionally substituted with one or more R₇; or
wherein R^{B} and R^{C} are taken together to form heterocyclyl optionally substituted with one or more R⁷;
each R⁷ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano.

2. The compound of claim 1, wherein each of R₁ and R₂ is independently hydrogen, C1-C6 alkyl, C1-C6 heteroalkyl, C1-C6 haloalkyl, —OR^{A}, fluorine, chlorine, bromine, iodine, nitro, or cyano.

3. The compound of claim 1, wherein each of R₁ and R₂ is independently hydrogen, fluorine, chlorine, bromine, iodine, or cyano.

4. The compound of claim 1, wherein each of R₁ and R₂ is independently hydrogen, fluorine, or chlorine.

5. The compound of claim 1, wherein R₁ is hydrogen and R₂ is fluorine, chlorine, bromine, iodine, nitro, or cyano.

6. The compound of claim 1, wherein R₂ is fluorine, chlorine, bromine, iodine, nitro, or cyano and R₂ is fluorine, chlorine, bromine, iodine, nitro, or cyano.

7. The compound of claim 1, wherein R₁ is hydrogen and R₂ is fluorine.

8. The compound of claim 1, wherein R₁ is hydrogen and R₂ is chlorine.

9. The compound of claim 1, wherein R₁ is chlorine and R₂ is fluorine.

10. The compound of claim 1, wherein each of R₁ and R₂ is independently fluorine.

11. The compound of claim 1, wherein R₅ is H.

12. The compound of claim 1, wherein R₆ is H.

13. The compound of claim 1, wherein R₃ is C1-C6 alkyl, C2-C6 alkenyl, C1-C6 alkynyl, C1-C6 heteroalkyl, C1-C6 haloalkyl, cycloalkyl, heterocyclyl, or N(R^{B})(R^{C}), each of which is optionally substituted with optionally substituted with one or more R₇.

14. The compound of claim 1, wherein R₃ is C1-C6 heteroalkyl optionally substituted with one or more R₇.

15. The compound of claim 1, wherein R₃ is C1-C6 haloalkyl, optionally substituted with one or more R₇.

16. The compound of claim 1, wherein R₃ is cycloalkyl, optionally substituted with one or more R₇.

17. The compound of claim 1, wherein R₃ is heterocyclyl, optionally substituted with one or more R₇.

18. The compound of claim 1, wherein R₃ is N(R^{B})(R^{C}).

19. The compound of claim 18, wherein R^{B} and R^{C} are taken together to form heterocyclyl optionally substituted with one or more R⁷ (e.g., a 4-membered heterocyclyl, 5-membered heterocyclyl, 6-membered heterocyclyl, or a bridged heterocyclyl).

20. The compound of claim 18, wherein R^{B} is hydrogen.

21. The compound of any one of claims 18-20, wherein R^{C} is C1-C6 alkyl, C2-C6 alkenyl, C1-C6 alkynyl, C1-C6 heteroalkyl, C1-C6 haloalkyl, cycloalkyl, heterocyclyl, each optionally substituted with one or more R₇.

22. The compound of any one of claims 18-21, wherein R^{C} is C1-C6 alkyl optionally substituted with one or more R₇.

23. The compound of any one of claims 18-21, wherein R^{C} is C1-C6 heteroalkyl optionally substituted with one or more R₇.

24. The compound of any one of claims 18-21, wherein R^{C} is C1-C6 haloalkyl optionally substituted with one or more R₇.

25. The compound of any one of claims 18-21, wherein R^{C} is cycloalkyl optionally substituted with one or more R₇ (e.g., monocyclic cycloalkyl, bicyclic cycloalkyl).

26. The compound of any one of claims 18-21, wherein R^{C} is heterocyclyl optionally substituted with one or more R₇.

27. The compound of any one of claims 21-26, wherein R₇ is C1-C6 alkyl, C2-C6 alkenyl, C1-C6 alkynyl, C1-C6 heteroalkyl, C1-C6 haloalkyl, —OR^{A}, fluorine, chlorine, bromine, iodine, nitro, or cyano.

28. The compound of claim 1, wherein the compound is a compound of Formula (I-a): 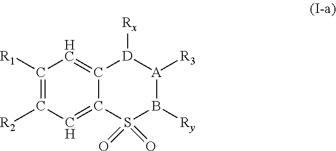
wherein:
A is carbon (C);
B and D are Nitrogen (N); and
wherein either A and B or A and D are connected via a double bond; and
wherein R₁ and R₂ are independently selected from the group consisting of hydrogen, fluorine, chlorine, bromine and iodine; and
wherein A and B form a double bond and Rₓ is H or wherein A and D form a double bond and R_{y} is H; and
wherein R₃ is independently selected from the group consisting of: 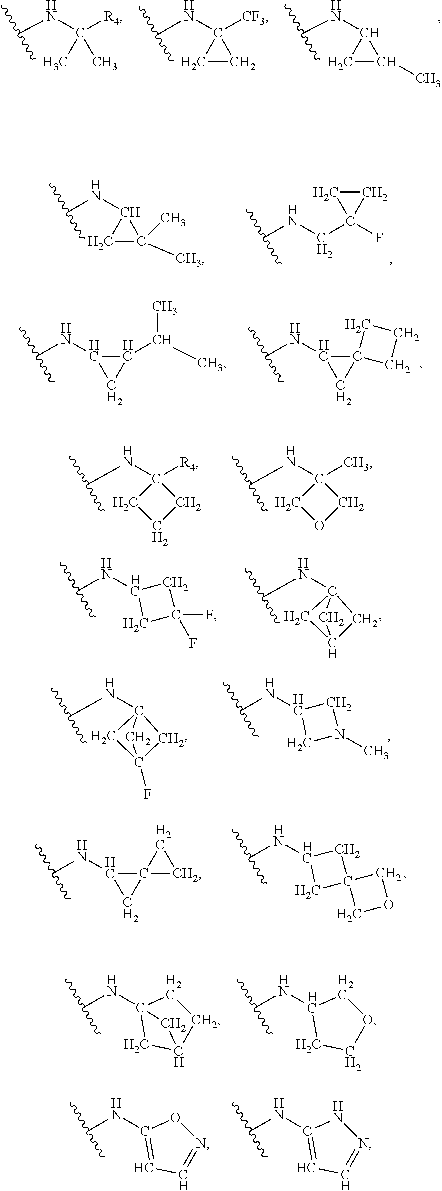 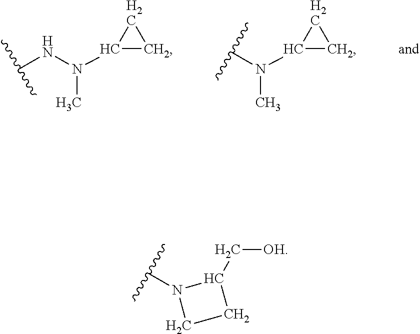
and wherein R₄ is selected from the group consisting of C1-C3 alkyl optionally substituted with one to two substituents selected from the group consisting of fluoro and hydroxy, ethynyl, C1-C2 alkoxy, (methoxy)-C1-C2 alkyl, cyano, fluoro, (methylsulfonyl)-C1-C2 alkyl, (dimethylamino)-C1-C2 alkyl and n-methyl carbamoyl.

29. The compound of claim 1, wherein:
A is carbon (C);
B and D are Nitrogen (N); and
wherein A and B or A and D are connected via a double or single bond; and
wherein R₁ and R₂ are independently selected from the group consisting of hydrogen, fluorine, chlorine, bromine and iodine; and
wherein A and B form a double bond and Rₓ is H or wherein A and D form a double bond and R_{y} is H; and
wherein R₃ is independently selected from the group consisting of:

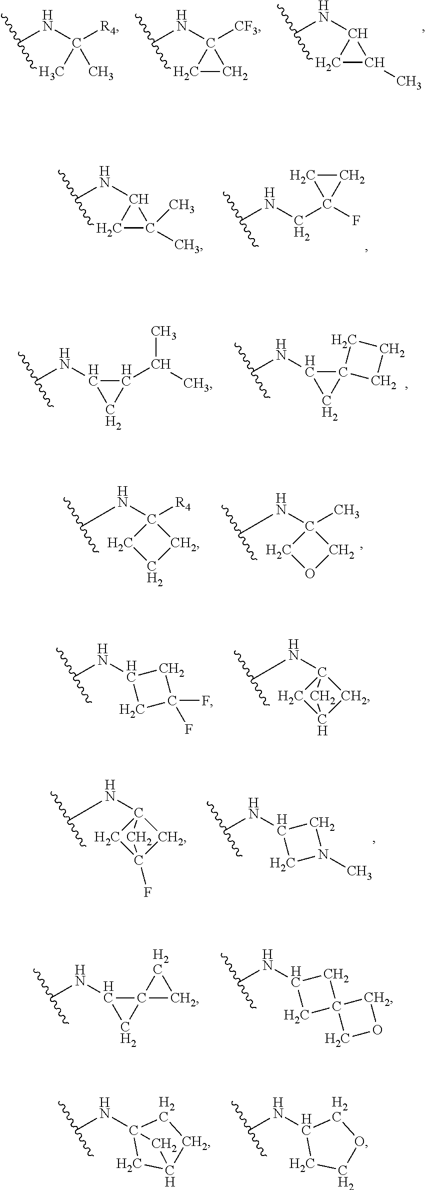

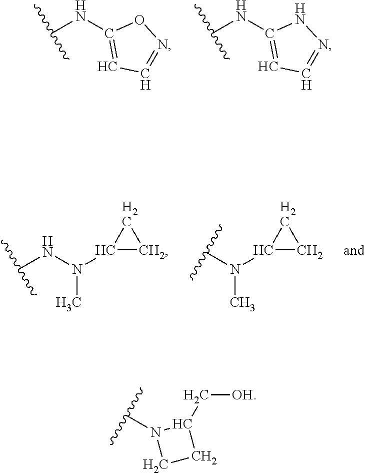

30. The compound of claim 1, wherein the compound is a compound of Formula (I-b): 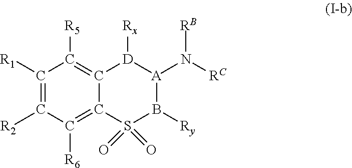
or a pharmaceutically acceptable salt, tautomer, stereoisomer, hydrate, or isotope thereof, wherein A, B, D, R₁, R₂, R₅, R₆, Rₓ R_{y}, R^{A}, R^{B}, R^{C}, and R⁷ are defined as provided for Formula (I).

31. The compound of claim 1, wherein the compound is a compound of Formula (I-c): 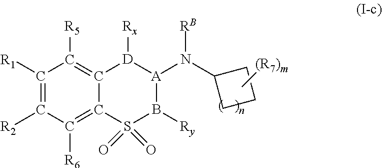
or a pharmaceutically acceptable salt, tautomer, stereoisomer, hydrate, or isotope thereof, wherein A, B, D, R₁, R₂, R₅, R₆, Rₓ R_{y}, R^{A} R^{B}, R^{C}, and R⁷ are defined as provided for Formula (I), m is an integer between 0 and 12; and n is an integer between 0 and 4.

32. The compound of claim 1, wherein the compound is a compound of Formula (I-e): 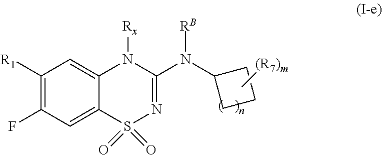
or a pharmaceutically acceptable salt, tautomer, stereoisomer, hydrate, or isotope thereof, wherein R₁, R₅, Rₓ, and R⁷ are defined as provided for Formula (I), m is an integer between 0 and 12; and n is an integer between 0 and 4.

33. The compound of claim 1, wherein the compound is a compound of Formula (I-f): 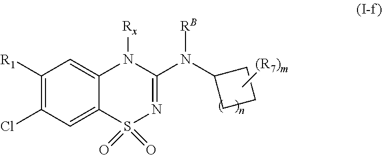
or a pharmaceutically acceptable salt, tautomer, stereoisomer, hydrate, or isotope thereof, wherein R₁, R₅, Rₓ, and R⁷ are defined as provided for Formula (I), m is an integer between 0 and 12; and n is an integer between 0 and 4.

34. The compound of claim 1, wherein the compound is selected from the group consisting of 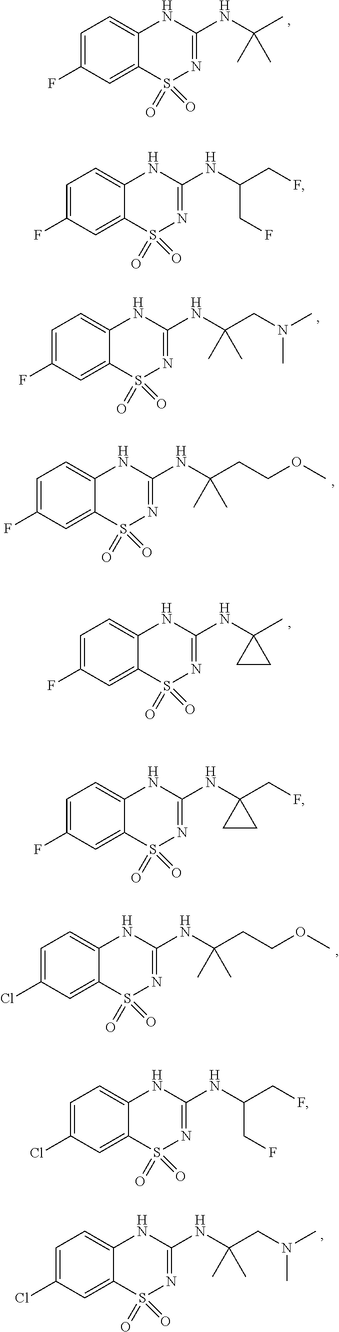 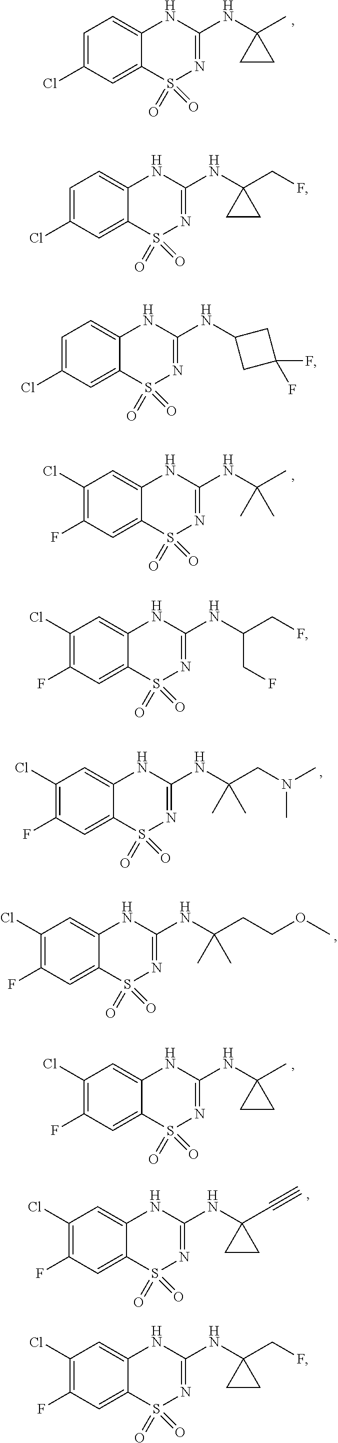 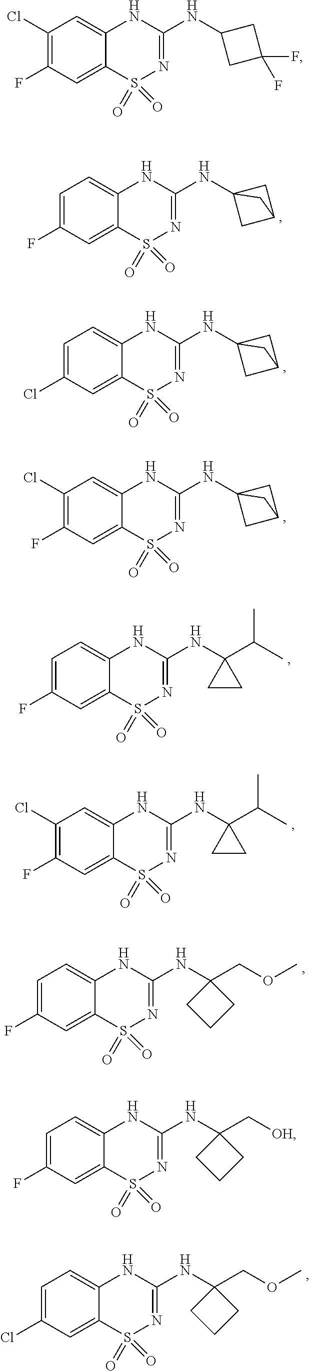 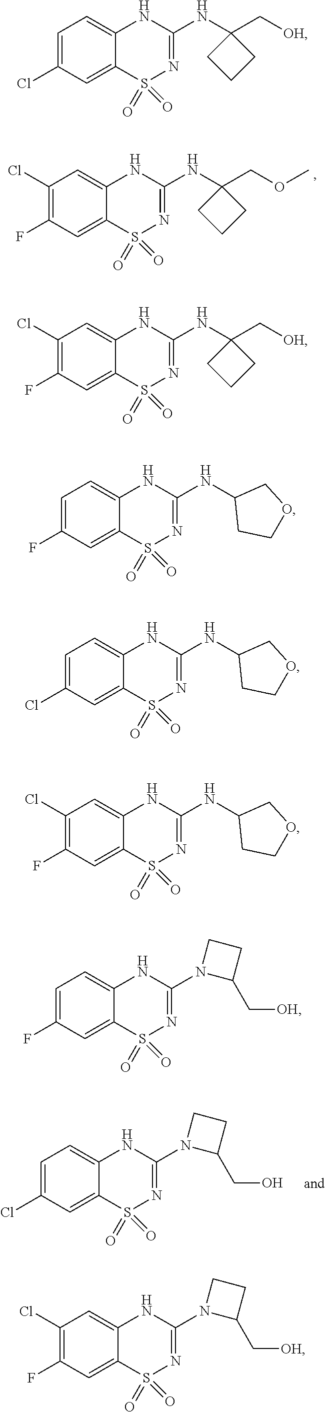
or a pharmaceutically acceptable salt thereof.

35. The compound of claim 1, wherein the compound is provided in Table 1 or a pharmaceutically acceptable salt thereof.

36. A pharmaceutical composition comprising a compound of claim 1 and a pharmaceutically acceptable excipient.

37. A method for treating a disease or disorder in a subject with a compound of claim 1, or a pharmaceutically acceptable thereof, or a pharmaceutical composition of claim 36.

38. The method of claim 37, wherein the disease or disorder is selected from a metabolic disorder (e.g., CHI), cancer, a neurological disorder, a cardiovascular disorder, a pulmonary disorder, an integumentary disorder, a sexual disorder, a urinary disorder, or a symptom thereof.

39. The method of claim 37, wherein the disease or disorder is a metabolic disorder.

40. The method of claim 37, wherein the metabolic disorder is hyperinsulinism, e.g., congenital hyperinsulinism (CHI).

41. The method of claim 37, wherein the subject has been diagnosed or identified with having the disease or disorder.

42. The method of claim 37, wherein the subject is a mammal (e.g., a human).

43. The method of claim 37, wherein the compound or pharmaceutical composition is formulated for oral or intravenous administration.

44. The method of claim 43, wherein the compound or pharmaceutical composition is a solid dosage form, in particular an oral dosage form, such as a tablet or capsule.

45. A process for preparing compounds according to any of the claims 1-6, wherein said process comprises the steps of:
a) providing a first compound according to formula (X) 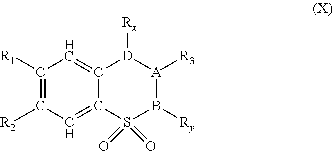
wherein:
A is carbon (C);
B and D are Nitrogen (N); and
wherein A and B or A and D are connected via a double or single bond; and
wherein R₁ and R₂ are independently selected from the group consisting of hydrogen, fluorine, chlorine, bromine and iodine; and
wherein A and B form a double bond, R_{y} is H or wherein A and D form a double bond, R_{y} is H and
wherein R₄ is independently selected from the group consisting of fluorine, chlorine, bromine and iodine, preferably chlorine:
b) providing a second compound according to formula H—R₃, wherein R₃ is selected from the group consisting of: 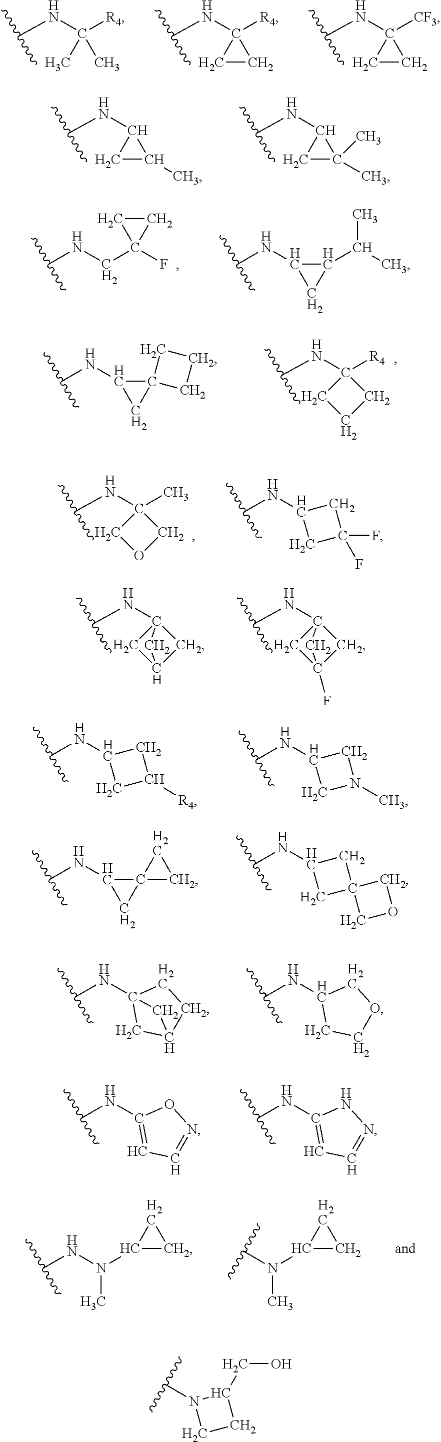
and wherein R₄ is selected from the group consisting of C1-C3 alkyl optionally substituted with one to two substituents selected from the group consisting of fluoro and hydroxy, ethynyl, C1-C2 alkoxy, (methoxy)-C1-C2 alkyl, cyano, fluoro, (methylsulfonyl)-C1-C2 alkyl, (dimethylamino)-C1-C2 alkyl and n-methyl carbamoyl;
c) reacting said first compound with said second compound under conditions that result in the formation of a compound of any one of the claims 1-35.
